(19) United States
(12) Patent Application Publication
(10) Pub. No.: US 2024/0254259 A1
(43) Pub. Date: Aug. 1, 2024

(54) DPEP-1 BINDING AGENTS AND METHODS OF USE

(71) Applicant: National Research Council Canada, Ottawa (CA)

(72) Inventors: Jamshid Tanha, Orleans (CA); Martin A. Rossotti, Ottawa (CA); Arthur Lau, Calgary (CA); Daniel Muruve, Calgary (CA)

(21) Appl. No.: 18/554,263

(22) PCT Filed: Apr. 8, 2022
(86) PCT No.: PCT/CA2022/050546

§ 371 Date: Oct. 6, 2023

Related U.S. Application Data
(60) Provisional application No. 63/172,530, filed on Apr. 8, 2021.

(51) Int. Cl. C07K 16/40; A61K 39/00; A61P 35/04

(52) U.S. Cl. CPC C07K 16/40; A61P 35/04; A61K 2039/505; C07K 2317/20; C07K 2317/21; C07K 2317/22; C07K 2317/569; C07K 2317/92

(57) ABSTRACT

DPEP-1 binding agents, including antibodies, and pharmaceutical compositions containing the same are described. Also provided are methods for using and manufacturing such binding agents, antibodies and pharmaceutical compositions, as well as methods of their use for treating or preventing a disorder in a human subject in need thereof.

### RELATED APPLICATION

This disclosure claims benefit of U.S. Provisional Patent Application Ser. No. 63/172,530, filed Apr. 8, 2021, incorporated herein by reference in its entirety.

### INCORPORATION OF SEQUENCE LISTING

The sequence listing that is contained in the file named “27929-P63463PC00_SequenceListing_2022-04-07”, which is 55,531 bytes and was created on Apr. 7, 2022, is filed herewith by electronic submission and is incorporated by reference herein.

### FIELD

Disclosed herein are DPEP-1 binding agents, including antibodies, and pharmaceutical compositions containing the same. Also disclosed are methods for using and manufacturing such agents, antibodies and pharmaceutical compositions, as well as methods and uses for treating or preventing a disorder in a human subject in need thereof.

### BACKGROUND

Inflammation is a host defense reaction to harmful stimuli which can be acute or chronic. Acute inflammation is characterized by redness, heat, swelling, and pain. The primary objectives of inflammation are to localize and eradicate the irritant and promote repair of the surrounding tissue. In most instances, inflammation is a necessary and beneficial process. The inflammatory response involves three major stages: first, dilation of arterioles to increase blood flow; second, microvascular structural changes and escape of plasma proteins from the bloodstream; and third, leukocyte transmigration through endothelium and accumulation at the site of injury. Leukocyte transendothelial migration (TEM) is a key step in their recruitment to sites of inflammation, injury, and immune reactions. The emigration of neutrophils to sites of inflammation is thought to require intercellular adhesion.

Failure to resolve the harmful stimuli prompting acute inflammation can lead to chronic inflammation, and some stimuli are likely to prompt immediate chronic inflammation. In some instances, inflammation results in secondary or chronic damage. Inflammation in a tumor microenvironment has also been implicated in cancer acceleration and tumor metastasis. The presence of pro-inflammatory molecules enables malignant cancer cells to adhere to the endothelial wall, leading to metastasis. Pro-inflammatory cytokines induce proliferation and aggregation of cancer cells, triggering other cancer cells to secrete more cytokines, resulting in a positive feedback loop. The role of adhesion molecules in acute and chronic inflammation is an area of study necessary for development of methods to control inflammation by modulating or blocking leukocyte adhesion to the endothelium.

Anti-inflammatory agents function as blockers, suppressors, or modulators of the inflammatory response. Tissue-specific control of inflammation is sometimes desirable to modulate inflammation in one tissue while maintaining the response in other tissues. Anti-inflammatory agents are used to treat various acute and chronic conditions. Most people have no trouble taking these agents, however some people develop side-effects which can be serious. In some groups, these medicines are prescribed with caution and only where there are no alternatives and at the lowest doses and durations necessary.

Hence, there remains a need for additional therapeutic compounds for reducing or blocking inflammation as current therapeutics, in particular because many of the current approaches cannot adequately treat some of the more extreme cases of inflammation. What is therefore needed are new compositions to function as blockers, suppressors, or modulators of the inflammatory response.

### SUMMARY

Using phage display library and panning strategy, single-domain antibodies (sdAB) were identified that bind to human DPEP-1 (hDPEP-1). These sdABs (sdABP01-09; SEQ ID NO: 1-9) were able to bind to hDPEP-1 in vitro and/or recognize HEK293T cells displaying hDPEP-1 on their surface. These human DPEP-1-specific V_{H}Hs were shown to have good thermostability, good SPR binding affinity, good dose response binding to cell-displayed hDPEP-1, and were characterized by epitope binding. Each of sdABP01-09 contains a Biotinylation Acceptor Peptide (BAP) and a polyhistidine (His6) tag. The core sequences of these sdABP01-09 without the BAP and His6 tag are shown in SEQ ID NOs: 12-20.

Disclosed herein are DPEP-1 binding agents, including antibodies, as well as pharmaceutical compositions comprising the same. Also disclosed are methods of using and making such DPEP-1 binding agents and compositions, as well as methods for screening for DPEP-1 binding agents.

In an aspect, disclosed herein is a binding agent that binds to DPEP-1 comprising:
(i) SEQ ID NOs: 21, 22, and 23;
(ii) SEQ ID NOs: 24, 25, and 26;
(iii) SEQ ID NOs: 27, 28, and 29;
(iv) SEQ ID NOs: 30, 31, and 32;
(v) SEQ ID NOs: 33, 34, and 35;
(vi) SEQ ID NOs: 36, 37, and 38;
(vii) SEQ ID NOs: 39, 40, and 41;
(viii) SEQ ID NOs: 42, 43, and 44; or
(ix) SEQ ID NOs: 45, 46, and 47.

In an embodiment, the binding agent comprises an antibody or antigen binding fragment thereof that binds to DPEP-1, wherein the antibody or antigen binding fragment thereof comprises an amino acid sequence that is at least 75%, 80%, 85%, 90%, 95%, 97%, 98%, 99%, 99.5%, 99.9% or 100% identical to any one of SEQ ID NOs: 12-20 and 48-56. In an embodiment, the binding agent comprises the amino acid sequence of any one of SEQ ID NOs: 12-20 and 48-56.

In an embodiment, the binding agent is monoclonal, polyclonal, chimeric, humanized antibody, or antigen binding fragment thereof.

In an embodiment, the binding agent is an antigen binding fragment fused to a Fc domain.

In an embodiment, the antigen binding fragment is a Fv, scFv, Fab, Fab′, F(ab′)2, dsFv, ds-scFv, sdAB, dimer, minibody, diabody, or multimer antigen binding fragment. In an embodiment, the antibody fragment is sdAB.

In an embodiment, the antibody or antigen binding fragment comprises one or more amino acids selected from the group consisting of D-amino acids, modified amino acids, amino acid analogs or combinations thereof.

In an embodiment, the modified amino acids comprise a modification selected from the group consisting of methylation, amidation, acetylation, and/or substitution with other chemical groups.

In an embodiment, the antibody or antigen binding fragment is modified by pegylation, acetylation, glycosylation, biotinylation, or prenylation.

In an embodiment, the antibody or antigen binding fragment is human, mouse, llama, rabbit, sheep, or goat antibody or antigen binding fragment thereof.

In a particular embodiment, the binding agent, antibody or antigen binding fragment is in a pharmaceutical composition further comprises a pharmaceutically acceptable carrier.

In another aspect, a method is disclosed for treating or preventing a disorder in a human subject in need thereof, comprising administering to the subject an effective amount of a binding agent, an antibody or antigen binding fragment thereof or pharmaceutical composition described in this disclosure. Also provided is a use of a binding agent, an antibody or antigen binding fragment therefore, or pharmaceutical composition described in this disclosure, for treating or preventing a disorder in a human subject in need thereof.

In an embodiment, the disorder is selected from the group consisting of acute kidney injury, sepsis-induced condition, and tumor metastasis. In an embodiment, the acute kidney injury comprises ischemia reperfusion-induced condition, pigment-induced condition, toxin-induced condition, or drug-induced condition. In an embodiment, the sepsis-induced condition comprises bacterial or viral sepsis-induced condition. In an embodiment, the viral sepsis-induced condition comprises a COVID-19 sepsis-induced condition. In an embodiment, the sepsis-induced condition is associated with acute respiratory distress syndrome, encephalopathy, liver failure, kidney failure, or heart failure.

In an embodiment, the tumor metastasis is associated with pancreatic cancer, kidney cancer, urogenital cancer, melanoma, prostate carcinoma, lung carcinoma, breast carcinoma, thyroid carcinoma, brain cancers, ovarian carcinomas, cervical cancer, uterine endometrial carcinoma, primary peritoneal carcinoma, mesothelioma, eye cancer, muscle, lymphoma, esophageal cancer, gastric cancer, liver cancer, small intestinal tumor, colon cancer, testicular cancer, skin cancers, or adrenal carcinoma. In an embodiment, the kidney cancer is renal cell carcinoma (RCC). In an embodiment, the urogenital cancer is urothelial carcinomas in urinary bladder, kidney, pelvic or ureter. In an embodiment, the lung carcinomas is non-small cell carcinoma, small cell carcinoma, or neuroendocrine carcinoma. In an embodiment, the neuroendocrine carcinoma is carcinoid tumor. In an embodiment, the breast carcinoma is ductal carcinoma, lobular carcinoma, or mixed ductal and lobular carcinoma. In an embodiment, the thyroid carcinomas is papillary thyroid carcinoma, follicular carcinoma, or medullary carcinoma. In an embodiment, the brain cancer is meningioma, astrocytoma, glioblastoma, cerebellum tumors, or medulloblastoma. In an embodiment, the ovarian carcinoma is serous, mucinous, or endometrioid type. In an embodiment, the cervical cancer is squamous cell carcinoma in situ, invasive squamous cell carcinoma, or endocervical adenocarcinoma. In an embodiment, the uterine endometrial carcinoma is endometrioid, serous, or mucinous type. In an embodiment, the mesothelioma is pleural or peritoneal. In an embodiment, the eye cancer is retinoblastoma. In an embodiment, the muscle cancer is rhabdosarcoma or leiomyosarcoma. In an embodiment, the esophageal cancer is adenocarcinoma or squamous cell carcinoma. In an embodiment, the gastric cancer is gastric adenocarcinoma or gastrointestinal stroma tumor. In an embodiment, the liver cancer is hepatocellular carcinoma or bile duct cancer. In an embodiment, the small intestinal tumor is small intestinal stromal tumor or carcinoid tumor. In an embodiment, the colon cancer is adenocarcinoma of the colon, colon high grade dysplasia, or colon carcinoid tumor. In an embodiment, the skin cancer is melanoma or squamous cell carcinoma.

In an embodiment, the disorder is selected from the group consisting of inflammation, ischemia-reperfusion injury, and ischemia-reperfusion injury related disorder. In an embodiment, the disorder is inflammation. In an embodiment, the inflammation comprises organ inflammation. In an embodiment, the inflammation is associated with an inflammatory disorder selected from the group consisting of gastritis, gout, gouty arthritis, arthritis, rheumatoid arthritis, kidney failure, lupus, asthma, psoriasis, pancreatitis, allergy, fibrosis, surgical complications, anemia, fibromyalgia, cancer, heart attack, congestive heart failure, stroke, aortic valve stenosis, arteriosclerosis, osteoporosis, multiple sclerosis, Alzheimer's disease, Parkinson's disease, ulcers, chronic bronchitis, asthma, allergy, acute lung injury, pulmonary inflammation, airway hyper-responsiveness, vasculitis, septic shock, inflammatory skin disorders, psoriasis, atopic dermatitis, eczema, and inflammatory bowel disease. In an embodiment, the inflammatory bowel disease is Crohn's disease or ulcerative colitis.

In an embodiment, the ischemia-reperfusion injury related disorder is associated with ischemic and post-ischemic events in organs and tissues, and the disorder is selected from a group consisting of thrombotic stroke, myocardial infarction, angina pectoris, embolic vascular occlusions, peripheral vascular insufficiency, splanchnic artery occlusion, arterial occlusion by thrombi, arterial by embolisms, arterial occlusion by non-occlusive processes, mesenteric arterial occlusion, mesenteric vein occlusion, ischemia-reperfusion injury to the mesenteric microcirculation, ischemic acute renal failure, ischemia-reperfusion injury to the cerebral tissue, intestinal intussusception, hemodynamic shock, tissue dysfunction, organ failure, restenosis, atherosclerosis, thrombosis, platelet aggregation, shock liver, spinal cord injury, or brain injury. In an embodiment, the arterial occlusion by non-occlusive processes is arterial occlusion following low mesenteric flow or sepsis. In an embodiment, the organ failure is heart failure, liver failure, kidney failure, or the like. In an embodiment, the ischemia-reperfusion injury is resulted from a surgical procedure. In an embodiment, the surgical procedure is peri-operative procedure, cardiac surgery, organ surgery, organ transplantation, angiography, cardiopulmonary, or cerebral resuscitation.

In an embodiment, the ischemia-reperfusion injury is associated with harvesting donor organs for transplantation. In an embodiment, the ischemia-reperfusion injury occurs to allograft organs during donor procurement, ex vivo handling, or implantation into a transplant recipient. In an embodiment, the subject in need thereof is an organ donor or organ recipient for transplantation.

In an embodiment, disclosed herein is a kit comprising a composition disclosed herein. In one embodiment, the kit further comprises a pharmaceutically acceptable carrier.

Other features and advantages of the present disclosure will become apparent from the following detailed description. It should be understood, however, that the detailed description, while indicating preferred implementations of the present disclosure, is given by way of illustration only, since various changes and modification within the spirit and scope of the disclosure will become apparent to those of skill in the art from the detailed description.

### BRIEF DESCRIPTION OF THE DRAWINGS

FIG. 1 shows thermal unfolding curves of V_{H}Hs. V_{H}H thermal unfolding midpoint temperatures (Tₘs) were determined using circular dichroism spectroscopy by following V_{H}H unfolding at 200 μg/mL concentration and 205 nm wavelength in 100 mM phosphate buffer pH 7.4. Raw data were converted to fraction (%) folded, as described in Example 2, and Tₘs (temperatures at the denaturation midpoint) were determined by Boltzmann curve fitting to plots of % folded vs temperature.

FIG. 2A shows surface plasma resonance (SPR) sensorgrams showing single-cycle kinetic analysis of V_{H}Hs binding to human DPEP-1. Recombinant human DPEP-1 was biotinylated as described in Example 3 and captured on CM5 sensorchip surfaces using Biotin CAPture reagent followed by flowing over surfaces V_{H}Hs at concentrations ranging from 0.625-10 nM (sdABP02), 2.5-40 nM (sdABP03/05/07), 6.25-100 nM (sdABP06) and 12.5-200 nM (sdABP01/04). Dark lines represent data points, light lines fit to the data. Data were generated in triplicates and data for one replicate set are shown.

FIG. 2B shows on-/off-rate maps summarizing V_{H}H kinetic rate constants, kₐs, kₐs, obtained in FIG. 2A. Diagonal lines represent equilibrium dissociation constants, K_{D}s. Data points are shown in triplicates.

FIG. 3A shows binding of DPEP-1-specific V_{H}Hs to cell-displayed human DPEP-1. Flow cytometry binding analyses of biotinylated V_{H}Hs at 100 nM concentrations against HEK-293T overexpressing human DPEP-1 (HEK-293T-hDPEP1+; profile on the right side of each graph). Clostridium difficile toxin A-specific A20.1 V_{H}H (Hussack et al., J Biol Chem. 286: 8961-8976, (2011)) was included as negative V_{H}H control (profile on the left side of each graph). Spectral shift to the right as seen in the case of DPEP-1-specific V_{H}Hs are indicative of binding to human DPEP-1. No binding was seen when indicated V_{H}Hs were tested against HEK293T-PARENTAL which is negative for hDPEP-1 expression (i.e. no spectral shift to the right).

FIG. 3B shows flow cytometry analysis of biotinylated V_{H}Hs. Dose response binding of human DPEP-1-specific V_{H}Hs to cell-displayed human DPEP-1 was determined. Flow cytometry analysis of biotinylated V_{H}Hs was performed at increasing V_{H}H concentrations against HEK-293-6E cells overexpressing human DPEP-1 (hDPEP-1). Clostridium difficile Toxin A-specific A20.1 V_{H}H (Hussack et al., J Biol Chem., 286: 8961-8976 [2011]) was included as negative V_{H}H control.

FIG. 3C shows groupings of graphs in FIG. 3B with similar maximal fluorescence plateau comparing with negative control A20.1 V_{H}H. V_{H}H concentrations at 50% binding, EC₅₀ values, were calculated from graphs and recorded in Table 5.

FIG. 3D shows immunoprecipitation of hDPEP-1 from HEK293-6E cells overexpressing hDPEP-1. A20.1 V_{H}H, specific for Clostridium difficile toxin A, was included as negative control. MW, protein molecular mass markers. hDPEP-1 ECD, hDPEP-1 extracellular domain.

FIG. 4A shows depiction summarizing epitope bins identified by SPR. sdABP01, sdABP06, sdABP02 and sdABP07 defined bins (i), (ii), (iii) and (iv), respectively, sdABP03 and 05 partially overlap bin (iv), while sdABP04 partially overlaps bin (iii).

FIG. 4B shows a heat map of epitope binning by ELISA. Competitive sandwich ELISA was performed to cluster V_{H}Hs by epitopes and represented as a heat map displaying all possible pair-wise combinations of V_{H}Hs (7×7=49). Binding pairs showing high binding signal (dark) were considered as recognizing non-overlapping epitopes hence belonging to different epitope bins or V_{H}H clusters, while those giving no or weak binding signals (light) were considered recognizing overlapping epitopes, thus belonging to the same epitope bins. A20.1 V_{H}H, specific for Clostridium difficile toxin A, included as negative control gives no signal as expected.

FIG. 5A shows representative immunofluorescent images of sdABP07 reducing monocytes infiltration (i.e. renal inflammation) in kidneys of LysM^{gfp/gfp} mice treated with LPS. Left panel: naïve; middle panel: LPS; right panel LPS+sdABP07.

FIG. 5B shows a graph representing renal inflammation quantified by number of adhered LysM⁺ monocytes found per field, in kidneys of LysM^{gfp/gfp} mice treated with LPS. sdABP07 reduced LPS-induced renal inflammation.

FIG. 6 shows SEC profiles of human DPEP-1 V_{H}H-Fcs. SEC profiles demonstrate V_{H}H-Fcs are free of aggregation and have elution volumes (Vₑs) consistent with their monomeric states. SEC was performed using a Superdex® 200 Increase column. mAU, milliabsorbance unit.

FIG. 7 shows binding profiles of VAH-Fcs to human DPEP-1 obtained by ELISA. All nine V_{H}H-Fcs (sdABP01-09) bound to human DPEP-1 in a dose dependent manner. Clostridium difficile toxin A-specific A20.1 V_{H}H-Fc (Hussack et al., J Biol Chem. 286: 8961-8976, [2011]) was included as negative V_{H}H control. Data represent the average of 3 independent experiments.

FIG. 8A shows results from assays on binding specificity and cross-reactivity of DPEP-1 V_{H}H-Fcs by flow cytometry. Flow cytometry binding analyses were performed at 125 nM V_{H}H-Fcs concentrations against HEK-293-6E cells overexpressing human DPEP-1. Clostridium difficile toxin A-specific A20.1 V_{H}H-Fc (Hussack et al., J Biol Chem. 286: 8961-8976, [2011]) was included as negative V_{H}H control, pAb is rabbit anti-human DPEP-1 polyclonal antibody positive control. Binding of V_{H}H-Fcs to cells was detected using anti-human:PE and that of pAb by using anti-rabbit:PE. Anti-human:PE and anti-rabbit:PE, denote negative binding assays where V_{H}H-Fcs and pAb were respectively omitted. Dotted line demarcates background signal. Measurements were done in quadruplicate.

FIG. 8B shows results from assays on binding specificity and cross-reactivity of DPEP-1 V_{H}H-Fcs by flow cytometry. Flow cytometry binding analyses were performed at 125 nM V_{H}H-Fcs concentrations against HEK-293-6E cells overexpressing mouse DPEP-1. Positive and negative controls, and secondary antibodies used were the same as in FIG. 8A. Dotted line demarcates background signal. Measurements were done in quadruplicate.

FIG. 8C shows results from assays on binding specificity and cross-reactivity of DPEP-1 V_{H}H-Fcs by flow cytometry. Flow cytometry binding analyses were performed at 125 nM V_{H}H-Fcs concentrations against HEK-293-6E cells overexpressing rat DPEP-1. Positive and negative controls, and secondary antibodies used were the same as in FIG. 8A. Dotted line demarcates background signal. Measurements were done in quadruplicate.

FIG. 8D shows results from assays on binding specificity and cross-reactivity of DPEP-1 V_{H}H-Fcs by flow cytometry. Flow cytometry binding analyses were performed at 125 nM V_{H}H-Fcs concentrations against HEK-293-6E cells overexpressing human DPEP-2. Positive and negative controls, and secondary antibodies used were the same as in FIG. 8A. Dotted line demarcates background signal. Measurements were done in quadruplicate.

FIG. 8E shows results from assays on binding specificity and cross-reactivity of DPEP-1 V_{H}H-Fcs by flow cytometry. Flow cytometry binding analyses were performed at 125 nM V_{H}H-Fcs concentrations against parental HEK-293-6E cells. Positive and negative controls, and secondary antibodies used were the same as in FIG. 8A. Dotted line demarcates background signal. Measurements were done in quadruplicate.

FIG. 9 shows dose response binding of V_{H}H-Fcs to human DPEP-1-expressing HEK293-6E cells obtained by flow cytometry. Calculated apparent EC₅₀s (EC₅₀apps) obtained from graphs are reported in Table 7. None of the V_{H}H-Fcs bound to parental, non-DPEP-1-expressing HEK293-6E cells at 125 nM (see FIG. 8E). Clostridium difficile Toxin A-specific A20.1 V_{H}H (Hussack et al., J Biol Chem., 286: 8961-8976 [2011]) was included as a negative V_{H}H control. Measurements were in triplicate.

FIG. 10 shows cross-reactivity assessment of sdABP05 and sdABP06 V_{H}H-Fcs against mouse DPEP-1 by flow cytometry. Dose response curves were obtained by titrating sdABP05 and sdABP06 against human DPEP-1-expressing (left) and mouse DPEP-1-expressing (right) HEK293-6E cells. sdABP05 and sdABP06 cross-reacted with mouse DPEP-1 and sdABP07 did not (see FIG. 8B). Calculated apparent EC₅₀s (EC₅₀apps) obtained from graphs are reported in Table 9. Measurements were in duplicate.

FIG. 11 shows epitope typing of human DPEP-1 V_{H}H-Fcs by SDS-PAGE/western blot against denatured recombinant DPEP1 from two different commercial sources. Presence of blots (binding signals) indicate sdABP05, sdABP06, sdABP03 and sdABP08 V_{H}H-Fcs recognize linear epitopes. The absence of binding signals for sdABP01, sdABP02, sdABP04, sdABP07 and sdABP09 VAH-Fc is an indirect indication these V_{H}H-Fcs recognize conformational epitopes (data not shown). Anti-human DPEP-1 polyclonal antibody (pAb; Proteintech, Cat #12222-1-AP) was included as reference. C, human DPEP1 (Creative biomart, Cat #DPEP1-77H); S, human DPEP1 (SinoBiologicals, Cat #13543-H08H). Images were acquired with 5 seconds (left panel) or 30 seconds (right panel) exposure times. Molecular weights (MW) are in kDa.

### DETAILED DESCRIPTION

### I. Definitions

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. Finally, terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

Where a term is provided in the singular, the inventors also contemplate aspects of the disclosure described by the plural of that term. As used in this specification and in the appended claims, the singular forms “a”, “an” and “the” include plural references unless the context clearly dictates otherwise, e.g., “an antibody” includes a plurality of antibodies. Thus, for example, a reference to “a method” includes one or more methods, and/or steps of the type described herein and/or which will become apparent to those persons skilled in the art upon reading this disclosure.

The term “administer”, “administering” or “administered” means the act of giving an agent or therapeutic treatment to a physiological system (e.g., a subject or in vivo, in vitro, or ex vivo cells, tissues, and organs).

The term “affinity”, as used herein, refers to the strength of the sum total of noncovalent interactions between a single binding site of a molecule and its binding partner. Unless indicated otherwise, as used herein, “binding affinity” refers to intrinsic binding affinity which reflects a 1:1 interaction between members of a binding pair. The affinity of a molecule X for its partner Y can generally be represented by the equilibrium dissociation constant (K_{D}). Affinity can be measured by common methods known in the art.

The term “amino acid” refers to naturally occurring amino acids, as well as non-naturally occurring or non-standard amino acids such as amino acid analogs, synthetic amino acids, and amino acid mimetics. These amino acids may be in the L- or D-(isomeric) configuration, or may include both dextrorotary forms. Amino acids that have been incorporated into antibodies are termed “residues”. Amino acids may be referred to herein by either the commonly known three letter symbols or by the one-letter symbols recommended by the IUPAC-IUB Biochemical Nomenclature Commission. The following amino acid definitions are used throughout the specification: Alanine: Ala (A) Arginine: Arg (R) Asparagine: Asn (N) Aspartic acid: Asp (D) Cysteine: Cys (C) Glutamine: Gln (Q) Glutamic acid: Glu (E) Glycine: Gly (G) Histidine: His (H) Isoleucine: Ile (I) Leucine: Leu (L) Lysine: Lys (K) Methionine: Met (M) Phenylalanine: Phe (F) Proline: Pro (P) Serine: Ser (S) Threonine: Thr (T) Tryptophan: Trp (W) Tyrosine: Tyr (Y) Valine: Val (V).

The term “binding agent”, as used herein, refers to a ligand, including an antibody or an antigen binding fragment thereof, that forms a complex with a receptor. The ligand may be selective or non-selective. The ligand may be an agonist (partial or full), antagonist (i.e., blocks the action of an agonist), an inverse agonist (i.e., exerts the opposite action of an agonist) or an allosteric modulator. Antagonists may be competitive (i.e., bind at the same site as the agonist) or non-competitive antagonists (i.e., binding permanently at the same site as the agonist or binding at an allosteric site—a site other than the active site). In some embodiments, the binding agent, antibody, or antigen binding fragment thereof, or composition in the present disclosure binds to and/or inhibits DPEP-1. In some embodiments, the binding agent, antibody, or antigen binding fragment thereof, or composition in the present disclosure reduces DPEP-1-regulated function. In some embodiments, the DPEP-1-regulated function comprises leukocyte recruitment, inflammation and tumor cell adhesion. In some embodiments, the binding agent, antibody, or antigen binding fragment thereof, or composition in the present disclosure does not affect DPEP-1 dipeptidase activity and/or its role in regulating tubular transport.

The term “diagnosed”, “diagnostic” or “diagnosis” means identifying the presence or nature of a pathologic condition. Diagnostic methods include observations and assays, and differ in their sensitivity and specificity. The “sensitivity” of a diagnostic observation or assay is the percentage of diseased individuals who test positive (percent of “true positives”). Diseased individuals not detected by the observation or assay are “false negatives.” Subjects who are not diseased and who test negative in the observation or assay are termed “true negatives.” The “specificity” of a diagnostic observation or assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

As used herein, the term “effective amount” refers to the amount of a therapy (e.g. a prophylactic or therapeutic agent) which is sufficient to effect beneficial or desired results, including clinical results. An effective amount can be administered in one or more administrations.

As used herein, the term “inflammatory disease” refers to diseases (treatable or preventable with compounds described herein) including, but not limited to, a) leukocyte recruitment, adhesion or activation and other disorders that involve neutrophils, monocytes, lymphocytes or macrophages, b) diseases involving the pathological production of inflammatory cytokines (e.g. TNF-α, interleukin (IL)-1β, IL-2, IL-6), and/or c) activation of nuclear factors that promote transcription of genes encoding inflammatory cytokines. Examples of these nuclear transcription factors include but are not restricted to: nuclear factor-κB (NFκB), activated protein-1 (AP-1), nuclear factor of activated T cells (NFAT).

The term “ischemia reperfusion injury”, as used herein, refers to the damage caused first by restriction of the blood supply to a tissue (ischemia) followed by a resupply of blood (reperfusion) and the attendant generation of free radicals, inflammation and cell death resulting in organ injury and dysfunction. In transplantation scenarios, ischemia reperfusion injury negatively affects allograft function.

The term “isolated”, as used herein, refers to a material is removed from its original environment (e.g., the natural environment, if it is naturally occurring). For example, a naturally-occurring antibody present in a living animal is not isolated, but the same antibody, separated from some or all of the coexisting materials in the natural system, is isolated. In some embodiments, the antibodies of the present disclosure are isolated antibodies.

The term “pharmaceutically acceptable carrier” refers to any such carriers known to those skilled in the art to be suitable for the particular mode of administration. For example, the term “pharmaceutically acceptable carrier” includes any and all solvents, dispersion media, coatings, antibacterial and antifungal agents, isotonic and absorption delaying agents and the like, that may be used as a media for a pharmaceutically acceptable substance. In addition, the active materials can also be mixed with other active materials that do not impair the desired action, or with materials that supplement the desired action, or have another action.

The term “pharmaceutically acceptable salt” as used herein refers to salts which are known to be non-toxic and are commonly used in the pharmaceutical literature. Typical inorganic acids used to form such salts include hydrochloric, hydrobromic, hydroiodic, nitric, sulfuric, phosphoric, hypophosphoric, and the like. Salts derived from organic acids, such as aliphatic mono and dicarboxylic acids, phenylsubstituted alkanoic acids, hydroxyalkanoic and hydroxyalkandioic acids, aromatic acids, aliphatic and aromatic sulfonic acids, may also be used. Such pharmaceutically acceptable salts include acetate, phenylacetate, trifluoroacetate, acrylate, ascorbate, benzoate, chlorobenzoate, dinitrobenzoate, hydroxy benzoate, methoxy benzoate, methylbenzoate, o-acetoxy benzoate, naphthalene-2-benzoate, bromide, isobutyrate, phenylbutyrate, beta-hydroxy butyrate, chloride, cinnamate, citrate, formate, fumarate, glycolate, heptanoate, lactate, maleate, hydroxymaleate, malonate, mesylate, nitrate, oxalate, phthalate, phosphate, monohydro genphosphate, dihydrogenphosphate, metaphosphate, pyrophosphate, propionate, phenylpropionate, salicylate, succinate, sulfate, bisulfate, pyrosulfate, sulfite, bisulfite, sulfonate, benzenesulfonate, p-bromophenylsulfonate, chlorobenzenesulfonate, ethanesulfonate, 2-hydroxyethanesulfonate, methanesulfonate, naphthalene-1-sulfonate, naphthalene-2-sulfonate, p-toluenesulfonate, xylenesulfonate, tartarate, and the like.

The term “prevent” or the equivalent, e.g., “prevention” or “preventing”, refers to reducing the occurrence of the disorder or condition in the treated sample relative to an untreated control sample, or delaying the onset or reducing the severity of one or more symptoms of the disorder or condition relative to the untreated control sample. As used herein, preventing ischemia-reperfusion injury includes preventing oxidative damage or preventing mitochondrial permeability transitioning, thereby preventing or ameliorating the harmful effects of the loss and subsequent restoration of blood flow to an effected organ. Preventing does not mean that a subject never develops the condition later in life, but that the probability of occurrence is reduced.

The terms “reducing,” “reduce,” or “reduction” in the context of a disease or condition herein refers to a decrease in the cause, symptoms, or effects of a disease or condition. Therefore, in the disclosed methods and uses, “reducing” can refer to a 10%, 20%, 30%, 40%, 50%, 60%, 70%, 80%, 90%, or 100% decrease, or any value or range there between, in the amount of injury due to reperfusion.

The term “subject” or “patient” or synonym thereto, as used herein includes all members of the animal kingdom, especially mammals, including human. The subject or patient is suitably a human.

The term “transplantation” is meant a surgical procedure by which a cell, tissue or organ is transferred from a donor subject to a recipient subject or from one part of the body to another in the same subject. The “donor subject” or “donor” is the subject who gives blood, cells, tissues, or an organ for another subject by blood transfusion or an organ transplant. The donor subject is a human or another mammal. The “recipient subject” or “recipient” is the subject who receives blood, cells, tissues, or an organ from another subject by blood transfusion or an organ transplant.

As used herein, the terms “treat”, “treatment” and “treating” refer to the prevention, reduction or amelioration of the progression, severity, and/or duration of at least one pathology and/or symptom of any condition or disease. The term “treatment” or “treating” refers to any administration or use of a compound disclosed herein and includes (i) inhibiting the disease, or the disease state in an individual that is experiencing or displaying the pathology or symptomatology of the disease, or the disease state (i.e., arresting further development of the pathology and/or symptomatology) or (ii) ameliorating the disease in an individual that is experiencing or displaying the pathology or symptomatology of the disease, or the disease state (i.e., reversing the pathology and/or symptomatology). The term “controlling” includes preventing, treating, eradicating, ameliorating or otherwise reducing the severity of symptoms of the disease, or the disease state.

As relating to inflammation, the terms “treat”, “treatment” and “treating” refer to the reduction or amelioration of the progression, severity, and/or duration of inflammation, or one or more symptoms thereof that results from the administration or use of one or more therapies (e.g., one or more prophylactic and/or therapeutic agents described herein). “Treatment” can also mean prolonging survival as compared to expected survival if not receiving treatment. Standard methods can be used to measure the magnitude of this effect, such as in vitro assays with purified enzyme, cell-based assays, animal models, or human testing. In exemplary embodiments, treatment of inflammation comprises one or more of reducing or ameliorating the progression, severity, and/or duration of inflammation associated with an inflammatory disorder selected from the group consisting of gastritis, gout, gouty arthritis, arthritis, rheumatoid arthritis, kidney failure, lupus, asthma, psoriasis, pancreatitis, allergy, fibrosis, surgical complications, anemia, fibromyalgia, cancer, heart attack, congestive heart failure, stroke, aortic valve stenosis, arteriosclerosis, osteoporosis, multiple sclerosis, Alzheimer's disease, Parkinson's disease, ulcers, chronic bronchitis, asthma, allergy, acute lung injury, pulmonary inflammation, airway hyper-responsiveness, vasculitis, septic shock, inflammatory skin disorders, psoriasis, atopic dermatitis, eczema, and inflammatory bowel disease such as Crohn's disease and ulcerative colitis. Sepsis is an inflammatory immune response triggered by an infection, for example, bacterial or viral infection. In some embodiments, treatment of inflammation comprises reducing or ameliorating the progression, severity, and/or duration of inflammation during sepsis in a subject. In some embodiments, the sepsis comprises bacterial or viral sepsis. In some embodiments, the viral sepsis comprises COVID-19-induced sepsis. Inflammation can also be associated with ischemia-reperfusion or acute kidney injury. In some embodiments, treatment of inflammation comprises inflammation that is associated with ischemia-reperfusion injury or acute kidney injury. Treatment of inflammation can also involve reducing inflammation or modifying the inflammatory profile of a tissue, e.g., into the lung tissue, liver tissue or kidney tissue. In some embodiments, treatment of inflammation comprises inflammation in lung tissue, liver tissue, or kidney tissue.

As relating to cancer, the terms “treat”, “treatment” and “treating” refer to the reduction or amelioration of the progression, severity, and/or duration of cancer, particularly a solid tumor, or one or more symptoms thereof that results from the administration or use of one or more therapies (e.g., one or more prophylactic and/or therapeutic agents). In exemplary embodiments, treatment of a solid tumor refers to one or more of (i) reducing the number of cancer cells; (ii) increasing tumor cell apoptosis; (iii) reducing tumor size; (iv) reducing tumor volume; (v) inhibiting, retarding, slowing to some extent, and/or stopping cancer cell infiltration into peripheral organs; (vi) inhibiting (e.g., slowing to some extent or stopping) tumor metastasis; (vii) inhibiting tumor growth; (viii) preventing or delaying occurrence and/or recurrence of a tumor; (ix) reduction of a cancer marker that is associated with the presence of cancer; and/or (ix) relieving to some extent one or more of the symptoms associated with the cancer. “Treatment” can also mean prolonging survival as compared to expected survival if not receiving treatment. Standard methods can be used to measure the magnitude of this effect, such as in vitro assays with purified enzyme, cell-based assays, animal models, or human testing. For example, an immunohistochemical analysis of a cancer tumor of the patient may show a significant increase in tumor cell apoptosis when the composition disclosed herein is administered to the patient or for use in a subject. When referring to a type of cancer that normally manifests as a solid tumor, a “clinically detectable” tumor is one that is detectable on the basis of tumor mass; e.g., by procedures such as CAT scan, MR imaging, X-ray, ultrasound or palpation, and/or which is detectable because of the expression of one or more cancer-specific antigens in a sample obtainable from a patient.

The recitation of numerical ranges by endpoints herein includes all numbers and fractions subsumed within that range (e.g. 1 to 5 includes for example 1, 1.5, 2, 2.75, 3, 3.90, 4, and 5). It is also to be understood that all numbers and fractions thereof are presumed to be modified by the term “about”.

### II. DPEP-1

DPEP-1, also known as renal dipeptidase, microsomal dipeptidase, or dehydropeptidase-1 and currently classified as EC 3.4.13.19 (previously EC 3.4.13.11), is a plasma membrane glycosyl phosphatidylinositol-anchored glycoprotein (Keynan et al., in Hooper (Ed.) Zinc Metalloproteases in Health and Disease Taylor and Francis, London pages 285-309 (1996), which is herein incorporated by reference). This zinc metalloprotease, which is expressed mainly in lung, liver and kidney including kidney endothelium and kidney proximal tubular brush border (Chaudhury et al, Cell 178(5), 1205-1221 (2019); herein incorporated by reference), is involved in vivo in renal metabolism of glutathione and in pulmonary metabolism of peptidyl leukotrienes. In addition, DPEP-1 is the only known example of a mammalian beta-lactamase and is also involved in the metabolism of glutathione and its conjugates, as well as leukotriene D4. DPEP-1 forms a disulfide-linked homodimer, with the molecular weight of the monomer ranging from about 48 to 59 kDa depending on the species of origin (Keynan et al., Biochem. 35:12511-12517 (1996), which is herein incorporated by reference; see, also, Example IVB).

Dipeptidase expression has been detected in several tissues although it is expressed mainly in lung, liver, and kidney. There have been reports of low levels of DPEP-1 activity in total extracts from spleen, small intestine and brain, while others have found no detectable activity in these organs. In the mouse, four distinct DPEP-1 mRNAs are present, and they are differentially expressed in several organs (Habib et al., J. Biol. Chem. 271:16273-16280 (1996)). Organ-specific differences in the nature and extent of pig DPEP-1 N-linked glycosylation also have been reported (Hooper et al., Biochem. J. 324:151-157 (1997)).

The level of DPEP-1 activity is highest in kidney and lung (Hirota et al., Eur. J. Biochem. 160:521-525 (1986); Habib et al., Proc. Natl. Acad. Sci. USA 95:4859-4863 (1998)). In the kidney, DPEP-1 expression is restricted to epithelial cells in the brush border region of the proximal tubules and the endothelial cells of the peritubular capillaries. In the lung, DPEP-1 expression has been detected in many cell types including endothelial cells as well as epithelial cells of the conducting airways, alveolar ducts, capillaries, and the basement membrane of alveoli and terminal bronchioles (Habib et al., supra, 1996); Inamura et al., Prostaglandins Leukotrienes and Essential Fatty Acids 50:85-92 (1994)). DPEP-1 expression also has been observed on endothelial cells of submucosal microvessels in the human trachea (Yamaya et al., Resp. Physiol. 111:101-109 (1998)). DPEP-1 expression pattern in the lung correlates with the strong lung homing of another DPEP-1 binding peptide, such as GFE-1 (Rajotte & Ruoslahti, Journal of Biological Chemistry, 274(17), 11593-11598 (1999)).

The DPEP-1 receptor functions as a leukocyte adhesion molecule or a tumor cell adhesion molecule expressed on vascular endothelium, or other parenchymal cells such as the kidney tubular epithelium (see Choudhury et al. 2019, “Dipeptidase-1 is an adhesion receptor for neutrophil recruitment in lungs and liver” Cell 178, 1205-1221; Lau, A, et al. “Dipeptidase-1 governs renal inflammation during ischemia reperfusion injury.” Science advances 8.5 (2022): eabm0142). Adhesion molecules are involved in the recruitment process, which are surface bound glycoprotein molecules expressed on leukocytes and/or endothelial cells. A key step in leukocyte recruitment is firm adhesion of leukocytes on the surface of the endothelium, which positions the leukocyte to migrate into the vessel wall through a sequence of adhesion and activation events to exerts its effects on the inflamed site. DPEP-1 could also function as an innate immune receptor during organ injury or infection. One such detection system is the so-called pattern recognition receptors (PRR) to detect key molecular signatures of invading pathogens, i.e., pathogen-associated molecular patterns (PAMPS), or endogenous damage-associated molecular patterns (DAMPs) thereby triggering the innate immune system (Janeway, C, et al., Annu. Rev. Immunol, 20 (2002), pp. 197-216). Examples of PRR are toll-like receptors (TLRs), which detect bacterial or viral products such as LPS, (TLR4) or peptidoglycans (TLR2)(Bell, J. K. et al., Trends Immunol, 24 (2003), pp. 528-533).

TLRs are transmembrane receptors that recognize pathogen-associated molecular patterns (PAMPs) through leucine-rich repeats (LRRs) in their extracellular domains that are implicated in ligand binding and auto-regulation (Kawai et al, Cell Death Differ. 13, 816-825, 2006). TLRs recognize microbial structures in the earliest phase of the host defense response and induce the expression of many immune and inflammatory genes, the products of which are tailored to drive the immune mechanisms necessary for eliminating the invading pathogen. TLRs have also been implicated in the recognition of damage-associated molecular patterns (DAMPs) and are becoming increasingly recognized as regulators of tumor-promoting inflammation and promoters of tumor survival signals. Other activators of such cellular pathways may provide effective therapeutic targets to treat pathogen and damage-associated cellular inflammation.

As used herein, the terms “dipeptidase”, and “membrane dipeptidase” are synonymous with “DPEP-1” and refers to the enzyme currently classified as EC 3.4.13.19 (previously EC 3.4.13.11) and also known as renal or microsomal dipeptidase or dehydropeptidase-1.

The term “selectively inhibits”, as used herein in reference to a DPEP-1 enzymatic activity, means that the binding agent decreases DPEP-1 activity in a manner that is selective for the DPEP-1 enzyme as compared to related but different enzymes such as other proteases. Thus, an DPEP-1 binding agent is distinct from a non-specific inhibitor of, for example, zinc metalloproteases. Thus, a DPEP-1 binding agent can selectively decrease DPEP-1 activity while having little or no effect on the activity of, for example, dipeptidyl peptidase IV. In one embodiment, the binding agent is a competitive inhibitor to prevent binding to DPEP-1.

The term “selectively binds”, as used herein in reference to a DPEP-1 binding agent, including an antibody or antigen binding fragment, means that the binding agent decreases DPEP-1-mediated leukocyte recruitment in a manner that is selective for the DPEP-1 receptor as compared to related but different receptors. DPEP-1 binding agent also refers to decreasing DPEP-1-mediated leukocyte recruitment where the DPEP-1 acts as an adhesion molecule for leukocytes or tumor cells independent of its enzymatic activity. Thus, an DPEP-1 binding agent can selectively decrease DPEP-1-mediated leukocyte recruitment while having little or no effect on the activity of, for example, dipeptidyl peptidase IV. In one embodiment, the binding agent is a competitive or non-competitive inhibitor to prevent binding to DPEP-1.

In one embodiment, the DPEP-1 binding agent disclosed herein is an antagonist of the DPEP receptor, i.e., blocks or dampens a biological response by binding to and blocking the receptor so as to disrupt the interaction and inhibit the function of an agonist or inverse agonist. In a particular embodiment, the DPEP-1 binding agent is a competitive antagonist, i.e., competes with an agonist for the active site. In another particular embodiment, the DPEP-1 binding agent is a non-competitive antagonist, i.e., binds at a site other than the active site.

The term specific binding, as used herein, includes both low and high affinity specific binding. Specific binding can be exhibited, for example, by a low affinity DPEP-1 binding molecule having a K_{D} for membrane dipeptidase of about 10⁻⁴ M to about 10⁻⁷ M. Specific binding also can be exhibited by a high affinity DPEP-1 binding molecule, for example, a DPEP-1 binding molecule having a K_{D} for membrane dipeptidase of at least about 10⁻⁷ M, at least about 10⁻⁸ M, at least about 10⁻⁹ M, at least about 10⁻¹⁰ M, or at least about 10⁻¹¹ M or 10⁻¹² M or greater. A DPEP-1 binding antibody can have, for example, a K_{D} for membrane dipeptidase of about 2×10⁻⁵ M to 10⁻⁷ M, for example, a K_{D} of about 10⁻⁶ to 10⁻⁷ M, or from about 10⁻⁸ M to 10⁻¹⁰ M measured by SPR, or from about 10⁻⁹M to 5×10⁻⁷ M measured by flow cytometry. Both low and high affinity DPEP-1 binding molecules that selectively bind to lung or kidney endothelium can be useful in the methods described herein.

The term “antibody” as used herein is intended to include monoclonal antibodies, polyclonal antibodies, chimeric antibodies, humanized antibodies, and antigen binding fragments thereof. The antibody may be from recombinant sources and/or produced in transgenic animals. The term “antigen binding fragment” or “antibody fragment” as used herein is intended to include without limitations Fv (a molecule comprising the VL and VH), single chain Fv (scFV; a molecule comprising the VL and VH connected by a peptide linker), Fab, Fab′, F(ab′)2, dsFv, ds-scFv, single domain antibodies (sdAB; molecules comprising a single variable domain having 3 or less CDRs, such as V_{H}H, V_{H}, V_{L}, and IgNAR antigen binding variable domain (VNAR)), and multivalent presentations of these. Also included are dimers, minibodies, diabodies, and multimers thereof, bi-specific and multi-specific antigen binding fragments, and domain antibodies. Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, dsFv, ds-scFv, sdAB, dimers, minibodies, diabodies, bispecific antigen binding fragments and other fragments can also be synthesized by recombinant techniques.

Antibodies to DPEP-1 may be prepared using techniques known in the art such as those described by Kohler and Milstein, Nature 256, 495 (1975) and in U.S. Patent Nos. RE 32,011; 4,902,614; 4,543,439; and 4,411,993, which are incorporated herein by reference. (See also Monoclonal Antibodies, Hybridomas: A New Dimension in Biological Analyses, Plenum Press, Kennett, McKearn, and Bechtol (eds.), 1980, and Antibodies: A Laboratory Manual, Harlow and Lane (eds.), Cold Spring Harbor Laboratory Press, 1988, which are also incorporated herein by reference). Within the context of the present disclosure, antibodies are understood to include monoclonal antibodies, polyclonal antibodies, chimeric antibodies, humanized antibodies, antigen binding fragments (e.g., Fab, and F(ab′)2) and recombinantly produced binding partners.

A single domain antibody (sdAB) has a single monomeric variable antibody domain. Similar to a whole antibody, it is able to bind selectively to a specific antigen. Because sdAB has a molecular weight of only 12-15 kDa, it is much smaller than conventional antibodies (150-160 kDa) which are composed of two heavy protein chains and two light chains, and even smaller than Fab fragments (˜50 kDa, one light chain and half a heavy chain) and single-chain variable fragments (˜25 kDa, two variable domains, one from a light and one from a heavy chain) (see Harmsen M M and De Haard H J (2007). “Properties, production, and applications of camelid single-domain antibody fragments”. Applied Microbiology and Biotechnology. 77 (1): 13-22; herein incorporated by reference). The first sdAB were engineered from heavy-chain antibodies found in camelids, which are called V_{H}H fragments. The Camelidae family includes camels and llamas. Single-domain camelid antibodies have been shown to be as specific as a regular antibody and are more robust in some cases. As well, they are easily isolated using the same phage panning procedure used for traditional antibodies, allowing them to be cultured in vitro in large concentrations. The smaller size and single domain make these antibodies easier to transform into bacterial cells for bulk production. sdABs are being researched for multiple pharmaceutical applications and have potential for use in the treatment of myriad of diseases including, but not limited to acute coronary syndrome, cancer and Alzheimer's disease. sdABs allow a broad range of applications in therapeutic use due to their small size, simple production and high affinity.

In the present disclosure, sdABs were generated by immunizing a male llama (Lama glama) with recombinant human DPEP-1 ectodomain (17-385) (hDPEP-1; accession number: P16444; NCBI Reference Sequence: NM_004413) using techniques known in the art (see, e.g. Baral T N, et al, (2013) Single-domain antibodies and their utility. Curr Protoc Immunol. 2013; 103:2-17; Henry K A, et al, (2016) Isolation of TGF-ββ-neutralizing single-domain antibodies of predetermined epitope specificity using next-generation DNA sequencing. Protein Eng Des Sel. 29:439-43; Henry K A, et al (2015) Identification of cross-reactive single-domain antibodies against serum albumin using next-generation DNA sequencing. Protein Eng Des Sel. 28:379-83; herein incorporated by reference). The animal was immunized subcutaneously three times with 200 μg of hDPEP-1 (days 0, 21, and 28). The priming immunization was adjuvanted with complete Freund's adjuvant and boost immunizations were adjuvanted with incomplete Freund's adjuvant. Blood samples were collected on days 0, 28, and 35, from which serum was obtained after clotting and peripheral blood mononuclear cells were purified by density gradient centrifugation. The resulting serum was specific to hDPEP-1 and was shown to have a strong, positive immune response against human DPEP-1 from two different sources (CreativeBioMart and SinoBiologicals).

Other methods are known in the art, for example, for producing polyclonal antibodies in a host, such as a rabbit or goat, by immunizing the host with the immunogen or immunogen fragment, generally with an adjuvant and, if necessary, coupled to a carrier; antibodies to the immunogen are collected from the sera. Further, the polyclonal antibody can be absorbed such that it is monospecific. That is, the sera can be absorbed against related immunogens so that no cross-reactive antibodies remain in the sera rendering it monospecific.

To produce monoclonal antibodies, antibody producing cells (lymphocytes) can be harvested from an immunized animal and fused with myeloma cells by standard somatic cell fusion procedures thus immortalizing these cells and yielding hybridoma cells. Such techniques are well known in the art, (e.g., the hybridoma technique originally developed by Kohler and Milstein (Continuous cultures of fused cells secreting antibody of predefined specificity. Nature 256:495-497, 1975) as well as other techniques such as the human B-cell hybridoma technique (Kozbor, D, and Roder, J: The production of monoclonal antibodies from human lymphocytes. Immunology Today 4:3 72-79, 1983), the EBV-hybridoma technique to produce human monoclonal antibodies (Cole et al. Monoclonal Antibodies in Cancer Therapy (1985) Allen R. Bliss, Inc., pages 77-96) and screening of combinatorial antibody libraries (Huse, W. D. et al, “Generation of a large combinatorial library of the immunoglobulin repertoire in phage lambda” Science 246:4935 1275-1282, 1989). Hybridoma cells can be screened immunochemically for production of antibodies specifically reactive with the protein or fragment thereof and the monoclonal antibodies can be isolated.

Chimeric antibody derivatives, i.e., antibody molecules that combine a non-human animal variable region and a human constant region are also contemplated within the scope of the disclosure. Chimeric antibody molecules can include, for example, the antigen binding domain from an antibody of a mouse, rat, llama, or other species, with human constant regions. Conventional methods may be used to make chimeric antibodies containing the immunoglobulin variable region which recognizes the target (See, for example, Morrison et al. (Chimeric Human Antibody Molecules: Mouse Antigen-Binding Domains with Human Constant Region Domains. PNAS 81:21 6851-6855, 1984), and Takeda et al. (Construction of chimaeric processed immunoglobulin genes containing mouse variable and human constant region sequences. Nature 314:452-454), and the patents of Cabilly et al., U.S. Pat. No. 4,816,567; Boss et al., U.S. Pat. No. 4,816,397; Tanaguchi et al., European Patent Publication EP171496; European Patent Publication 0173494, United Kingdom patent GB 2177096B).

Monoclonal or chimeric antibodies specifically reactive with a target as described herein can be further humanized by producing human constant region chimeras, in which parts of the variable regions, particularly the conserved framework regions of the antigen-binding domain, are of human origin and only the hypervariable regions are of non-human origin. Such immunoglobulin molecules may be made by techniques known in the art, (e.g., Teng et al. (Construction and Testing of Mouse—Human Heteromyelomas for Human Monoclonal Antibody Production. PNAS 80:12 7308-7312, 1983), Kozbor et al., supra; Olsson et al. (Methods in Enzymol, 92:3-16 1982) and PCT Publication WO92/06193 or EP 0239400). Humanized antibodies can also be commercially produced (Scotgen Limited, 2 Holly Road, Twickenham, Middlesex, Great Britain).

For producing recombinant antibodies (see generally Huston et al, 1991; Johnson and Bird, 1991; Mernaugh and Mernaugh, 1995), messenger RNAs from antibody producing B-lymphocytes of animals, or hybridoma are reverse-transcribed to obtain complementary DNAs (cDNAs). Antibody cDNA, which can be full or partial length, is amplified and cloned into a phage or a plasmid. The cDNA can be a partial length of heavy and light chain cDNA, separated or connected by a linker. The antibody, or antigen binding fragment, is expressed using a suitable expression system to obtain recombinant antibody. Antibody cDNA can also be obtained by screening pertinent expression libraries.

### III. DPEP-1 Binding Agents and Compositions

Binding or blocking DPEP-1 has utility in treating or preventing disorder in a human subject. For example, binding or blocking DPEP-1 has utility for reducing inflammation-mediated diseases in lung and kidney, such as during sepsis or acute kidney injury. Binding to or blocking DPEP-1 also has utility for reducing tumor metastasis. Disclosed herein are compositions that bind to or block DPEP-1, including, but not limited to, antibodies. DPEP-1 is a target for therapeutic intervention and it has role as a physical adhesion receptor for neutrophil sequestration independent of its enzymatic activity (see Choudhury, S. R, et al., Cell 178.5 (2019): 1205-1221; herein incorporated by reference). In particular, DPEP-1 is a major adhesion receptor on the lung and liver endothelium, and it is a therapeutic target for, e.g. neutrophil-driven inflammatory diseases of the lungs. DPEP-1 also acts as an adhesion receptor for neutrophils within the hepatic and pulmonary vasculature following stimulation by the bacterial endotoxin lipopolysaccharide (LPS) and represents a major neutrophil adhesion receptor identified on lung endothelium. Peptide LSALT is a binding agent of DPEP-1 which has been shown to be useful, through targeting DPEP-1, in inhibiting LPS-induced recruitment of neutrophils in the pulmonary vasculature, providing therapeutic benefits and increasing overall survival in endotoxemia murine models, abrogating ischemia reperfusion-induced acute kidney injury in mice, reducing tumor burden in animals injected with metastatic cancer cells (see Choudhury, S. R, et al., Cell 178.5 (2019): 1205-1221; Lau, A, et al. Science advances 8.5 (2022): eabm0142; US20200223888A1; U.S. Ser. No. 10/493,127B2; each of which herein incorporated by reference in its entirety). These form as scientific basis for predicting that the presently disclosed antibodies which bind to DPEP-1 have similar therapeutic effects, providing clinical benefits in a human subject in treating or preventing a disease or disorder described in the present disclosure.

### A. DPEP-1 Binding Antibodies

Disclosed herein are binding agents such as antibodies that bind to DPEP-1. Variants and modified embodiments of these binding antibodies that are capable of being used in these methods are also provided. In one embodiment, the antibodies modulate the activity of DPEP-1 and more particularly, inhibit the activity of DPEP-1 either competitively or non-competitively.

Using phase display library and panning strategy, single-domain antibodies (sdAB; also known as V_{H}H; i.e. sdABP01-09; SEQ ID NO: 1-9) were identified that bind to human DPEP-1 (hDPEP-1). These sdABs were able to recognize HEK293T cells displaying hDPEP-1 (sdABP01-07; SEQ ID NO: 1-7), shown to have good thermostability, good SPR binding affinity, good dose response binding to cell-displayed hDPEP-1, and were characterized by epitope binning (by SPR: sdABP01-07; by ELISA: sdABP01-04 and 07-09). Each of sdABP01-09 contains a Biotinylation Acceptor Peptide (BAP) and a His6 tag. The core sequences of these sdABP01-09 without the BAP and His6 tag are shown in SEQ ID NO: 12-20).

In an embodiment, the antigen binding fragment is a Fv, scFv, Fab, Fab′, F(ab′)2, dsFv, ds-scFv, sdAB, dimer, minibody, diabody, or multimer antigen binding fragment. In an embodiment, the antigen binding fragment is sdAB.

In some embodiments, the antibody or antigen binding fragment thereof that binds to DPEP-1 contains one or more modifications to increase protease resistance, serum stability and/or bioavailability. In some embodiments, the modification is selected from pegylation, acetylation, glycosylation, biotinylation, prenylation or substitution with D-amino acid and/or unnatural amino acid of the antibody or antigen binding fragment. The antibody or antigen binding fragment can contain one or more non-canonical disulphide linkages, e.g., at IMGT (ImMunoGeneTics) positions 54 and 78, to increase stability, protease resistance, serum stability and/or bioavailability (see Hussack, G et al. “Engineered single-domain antibodies with high protease resistance and thermal stability.” PloS ONE 6.11 (2011): e28218; herein incorporated by reference). Accordingly, in some embodiments, the antibody or antigen binding fragment thereof that binds to DPEP-1 contains one or more non-canonical disulphide linkages to increase stability, protease resistance, serum stability and/or bioavailability. In some embodiments, one or more non-canonical disulphide linkages are at IMGT positions 54 and 78.

In certain embodiments, the antibody or antigen binding fragment thereof that binds to DPEP-1 contains one or more L-amino acids, D-amino acids, and/or non-standard amino acids.

In various embodiments, the antibody or antigen binding fragment thereof that binds to DPEP-1 further comprises one or more amino acid residues or analogues at the C-terminus, the N-terminus or both the C-terminus and the N-terminus. In some embodiments, the activity bearing sequence of the antibody or antigen binding fragment is not appreciably impacted by the addition of these additional amino acid(s).

In another embodiment, the antibody or antigen binding fragment thereof that binds to DPEP-1 further comprises 1, 2, 3, 4, or 5 amino acid residues at the N-terminus.

In various embodiments, the antibody or antigen binding fragment is selected from X-antibody or -antigen binding fragment, XX-antibody or -antigen binding fragment, XXX-antibody or -antigen binding fragment, XXXX-antibody or -antigen binding fragment, or XXXXX-antibody or -antigen binding fragment, where X is any naturally-occurring amino acid or where X is an unconventional amino acid or amino acid analog as described herein and known to those of skill in the art.

In another embodiment, the antibody or antigen binding fragment thereof that binds to DPEP-1 further comprises 1, 2, 3, 4, or 5 amino acid residues at the C-terminus and is selected from antibody- or antigen binding fragment-X, antibody- or antigen binding fragment-XX, antibody- or antigen binding fragment-XXX, antibody- or antigen binding fragment-XXXX, or antibody- or antigen binding fragment-XXXXX where X is any naturally-occurring amino acid or where X is an unconventional amino acid or amino acid analog as described herein and known to those of skill in the art.

In one embodiment, the antibody or antigen binding fragment thereof that binds to DPEP-1 further comprises 1, 2, 3, 4, or 5 amino acid residues at the N-terminus and C-terminus of the antibody or antigen binding fragment and is selected from X-antibody or antigen binding fragment-X, X-antibody or antigen binding fragment-XX, X-antibody or antigen binding fragment-XXX, X-antibody or antigen binding fragment-XXXX, X-antibody or antigen binding fragment-XXXXX, XX-antibody or antigen binding fragment-X, XX-antibody or antigen binding fragment-XX, XX-antibody or antigen binding fragment-XXX, XX-antibody or antigen binding fragment-XXXX, XX-antibody or antigen binding fragment-XXXXX, XXX-antibody or antigen binding fragment-X, XXX-antibody or antigen binding fragment-XX, XXX-antibody or antigen binding fragment-XXX, XXX-antibody or antigen binding fragment-XXXX, XXX-antibody or antigen binding fragment-XXXXX, XXXX-antibody or antigen binding fragment-X, XXXX-antibody or antigen binding fragment-XX, XXXX-antibody or antigen binding fragment-XXX, XXXX-antibody or antigen binding fragment-XXXX, XXXX-antibody or antigen binding fragment-XXXXX, XXXXX-antibody or antigen binding fragment-X, XXXXX-antibody or antigen binding fragment-XX, XXXXX-antibody or antigen binding fragment-XXX, XXXXX-antibody or antigen binding fragment-XXXX, or XXXXX-antibody or antigen binding fragment-XXXXX, where X is any naturally-occurring amino acid or where X is an unconventional amino acid or amino acid analog as described herein and known to those of skill in the art.

Disclosed herein are compositions comprising an effective amount of an antibody or antigen binding fragment thereof that binds to DPEP-1, wherein the antibody or antigen binding fragment thereof that binds to DPEP-1 comprises any one of sdABP01, sdABP02, sdABP03, sdABP04, sdABP05, sdABP06, sdABP07, sdABP08, or sdABP09, or derivative.

In one embodiment, the antibody or antigen binding fragment thereof that binds to DPEP-1 comprises an amino acid sequence that is at least 75%, 80%, 85%, 90%, 95%, 97%, 98%, 99%, 99.5%, 99.9% or 100% identical to any one of SEQ ID NOs: 12-20 and 48-56. In one embodiment, the antibody or antigen binding fragment thereof that binds to DPEP-1 comprises an amino acid sequence that is at least 75%, 80%, 85%, 90%, 95%, 97%, 98%, 99%, 99.5%, 99.9% or 100% identical to any one of SEQ ID NO: 13, 15, or 18. In some embodiments, any differences in sequence are limited to framework regions.

As is understood in the art, the amino acid position or boundary delineating the CDR regions of an antibody can vary, depending on the context and the different definitions known in the art. Some positions within the variable regions can be viewed as hybrid CDRs in that the positions can be within a CDR region under one set of criteria while being deemed to be outside a CDR region under another set of criteria. In some embodiments, the CDRs in the foregoing variable light and variable heavy chains can be delineated using the IMGT, Kabat, Chothia, AbM, Contact, or Paratome schemes, or another scheme known in art. The “Kabat” approach for defining CDRs uses sequence variability and is most commonly used (Kabat et al., 1991, “Sequences of Proteins of Immunological Interest, 5th Ed.” NIH 1:688-96l; herein incorporated by reference). “Chothia” uses the location of structural loops (Chothia and Lesk, 1987. J Mol Biol. 196:901-17; Chothia et al., 1992, J. Mol. Biol. 227: 799-817; herein incorporated by reference). The IMGT numbering scheme is an adaptation of the numbering scheme of Chothia (Lefranc et al., 1999, Nucleic Acids Research, 27:209-212; http://imgt.cines.fr; herein incorporated by reference). CDRs defined by “AbM” is a compromise between the Kabat and Chothia, and is delineated using Oxford Molecular AbM antibody modeling software (see, Martin et al., 1989, PNAS, 86:9268; see also www.bioinf-org.uk/abs; herein incorporated by reference). The “Contact” CDR delineations are based on analysis of known antibody-antigen crystal structures (see, e.g., MacCallum et al., 1996, J. Mol. Biol. 262, 732-45). The “Paratome” approach involves computational programs based on a set of consensus regions derived from a structural alignment of a non-redundant set of known antibody-antigen complexes (Kunik et al., 2012, Nucl Acids Res. W521-4; see also www.ofranlab.org/paratome/; herein incorporated by reference). Although the CDRs described herein are based on IMGT definition, it is to be understood that CDRs based on other methods are to be encompassed herein. In some embodiments, the CDRs described herein are based on IMGT numbering.

Accordingly, the present disclosure provides a binding agent that binds to DPEP-1 comprising:
(i) SEQ ID NOs: 21, 22, and 23;
(ii) SEQ ID NOs: 24, 25, and 26;
(iii) SEQ ID NOs: 27, 28, and 29;
(iv) SEQ ID NOs: 30, 31, and 32;
(v) SEQ ID NOs: 33, 34, and 35;
(vi) SEQ ID NOs: 36, 37, and 38;
(vii) SEQ ID NOs: 39, 40, and 41;
(viii) SEQ ID NOs: 42, 43, and 44; or
(ix) SEQ ID NOs: 45, 46, and 47.

In some embodiments, the binding agent is an antibody or antigen binding fragment thereof. In some embodiments, the binding agent comprises three sequential CDRs having the amino acid sequences of SEQ ID NOs: 21, 22, and 23. In some embodiments, the binding agent comprises three sequential CDRs having the amino acid sequences of SEQ ID NOs: 24, 25, and 26. In some embodiments, the binding agent comprises three sequential CDRs having the amino acid sequences of SEQ ID NOs: 27, 28, and 29. In some embodiments, the binding agent comprises three sequential CDRs having the amino acid sequences of SEQ ID NOs: 30, 31, and 32. In some embodiments, the binding agent comprises three sequential CDRs having the amino acid sequences of SEQ ID NOs: 33, 34, and 35. In some embodiments, the binding agent comprises three sequential CDRs having the amino acid sequences of SEQ ID NOs: 36, 37, and 38. In some embodiments, the binding agent comprises three sequential CDRs having the amino acid sequences of SEQ ID NOs: 39, 40, and 41. In some embodiments, the binding agent comprises three sequential CDRs having the amino acid sequences of SEQ ID NOs: 42, 43, and 44. In some embodiments, the binding agent comprises three sequential CDRs having the amino acid sequences of SEQ ID NOs: 45, 46, and 47.

In one embodiment, the antibody or antigen binding fragment thereof that binds to DPEP-1 comprises an amino acid sequence that is at least 75%, 80%, 85%, 90%, 95%, 97%, 98%, 99%, 99.5%, 99.9% or 100% identical to SEQ ID NO: 12. In one embodiment, the antibody or antigen binding fragment thereof comprises the amino acid sequence of SEQ ID NO: 12. In one embodiment, the antibody or antigen binding fragment thereof that binds to DPEP-1 is an sdAB comprising at least one CDR having an amino acid sequence as set forth in any one of SEQ ID NOs: 21, 22, and 23. In one embodiment, the antibody or antigen binding fragment thereof that binds to DPEP-1 comprises an amino acid sequence that is at least 75%, 80%, 85%, 90%, 95%, 97%, 98%, 99%, 99.5%, 99.9% or 100% identical to SEQ ID NO: 12, wherein the antibody or antigen binding fragment thereof comprises three sequential CDRs comprising or consisting of amino acid sequences of SEQ ID NOs: 21, 22, and 23. In some embodiments, any differences in sequence are limited to framework regions.

In one embodiment, the antibody or antigen binding fragment thereof that binds to DPEP-1 comprises an amino acid sequence that is at least 75%, 80%, 85%, 90%, 95%, 97%, 98%, 99%, 99.5%, 99.9% or 100% identical to SEQ ID NO: 13. In one embodiment, the antibody or antigen binding fragment thereof comprises the amino acid sequence of SEQ ID NO: 13. In one embodiment, the antibody or antigen binding fragment thereof that binds to DPEP-1 is an sdAB comprising at least one CDR having an amino acid sequence as set forth in any one of SEQ ID NOs: 24, 25, and 26. In one embodiment, the antibody or antigen binding fragment thereof that binds to DPEP-1 comprises an amino acid sequence that is at least 75%, 80%, 85%, 90%, 95%, 97%, 98%, 99%, 99.5%, 99.9% or 100% identical to SEQ ID NO: 13, wherein the antibody or antigen binding fragment thereof comprises three sequential CDRs comprising or consisting of amino acid sequences of SEQ ID NOs: 24, 25, and 26. In some embodiments, any differences in sequence are limited to framework regions.

In one embodiment, the antibody or antigen binding fragment thereof that binds to DPEP-1 comprises an amino acid sequence that is at least 75%, 80%, 85%, 90%, 95%, 97%, 98%, 99%, 99.5%, 99.9% or 100% identical to SEQ ID NO: 14. In one embodiment, the antibody or antigen binding fragment thereof comprises the amino acid sequence of SEQ ID NO: 14. In one embodiment, the antibody or antigen binding fragment thereof that binds to DPEP-1 is an sdAB comprising at least one CDR having an amino acid sequence as set forth in any one of SEQ ID NOs: 27, 28, and 29. In one embodiment, the antibody or antigen binding fragment thereof that binds to DPEP-1 comprises an amino acid sequence that is at least 75%, 80%, 85%, 90%, 95%, 97%, 98%, 99%, 99.5%, 99.9% or 100% identical to SEQ ID NO: 14, wherein the antibody or antigen binding fragment thereof comprises three sequential CDRs comprising or consisting of amino acid sequences of SEQ ID NOS: 27, 28, and 29. In some embodiments, any differences in sequence are limited to framework regions.

In one embodiment, the antibody or antigen binding fragment thereof that binds to DPEP-1 comprises an amino acid sequence that is at least 75%, 80%, 85%, 90%, 95%, 97%, 98%, 99%, 99.5%, 99.9% or 100% identical to SEQ ID NO: 15. In one embodiment, the antibody or antigen binding fragment thereof comprises the amino acid sequence of SEQ ID NO: 15. In one embodiment, the antibody or antigen binding fragment thereof that binds to DPEP-1 is an sdAB comprising at least one CDR having an amino acid sequence as set forth in any one of SEQ ID NOs: 30, 31, and 32. In one embodiment, the antibody or antigen binding fragment thereof that binds to DPEP-1 comprises an amino acid sequence that is at least 75%, 80%, 85%, 90%, 95%, 97%, 98%, 99%, 99.5%, 99.9% or 100% identical to SEQ ID NO: 15, wherein the antibody or antigen binding fragment thereof comprises three sequential CDRs comprising or consisting of amino acid sequences of SEQ ID NOs: 30, 31, and 32. In some embodiments, any differences in sequence are limited to framework regions.

In one embodiment, the antibody or antigen binding fragment thereof that binds to DPEP-1 comprises an amino acid sequence that is at least 75%, 80%, 85%, 90%, 95%, 97%, 98%, 99%, 99.5%, 99.9% or 100% identical to SEQ ID NO: 16. In one embodiment, the antibody or antigen binding fragment thereof comprises the amino acid sequence of SEQ ID NO: 16. In one embodiment, the antibody or antigen binding fragment thereof that binds to DPEP-1 is an sdAB comprising at least one CDR having an amino acid sequence as set forth in any one of SEQ ID NOs: 33, 34, and 35. In one embodiment, the antibody or antigen binding fragment thereof that binds to DPEP-1 comprises an amino acid sequence that is at least 75%, 80%, 85%, 90%, 95%, 97%, 98%, 99%, 99.5%, 99.9% or 100% identical to SEQ ID NO: 16, wherein the antibody or antigen binding fragment thereof comprises three sequential CDRs comprising or consisting of amino acid sequences of SEQ ID NOs: 33, 34, and 35. In some embodiments, any differences in sequence are limited to framework regions.

In one embodiment, the antibody or antigen binding fragment thereof that binds to DPEP-1 comprises an amino acid sequence that is at least 75%, 80%, 85%, 90%, 95%, 97%, 98%, 99%, 99.5%, 99.9% or 100% identical to SEQ ID NO: 17. In one embodiment, the antibody or antigen binding fragment thereof comprises the amino acid sequence of SEQ ID NO: 17. In one embodiment, the antibody or antigen binding fragment thereof that binds to DPEP-1 is an sdAB comprising at least one CDR having an amino acid sequence as set forth in any one of SEQ ID NOs: 36, 37, and 38. In one embodiment, the antibody or antigen binding fragment thereof that binds to DPEP-1 comprises an amino acid sequence that is at least 75%, 80%, 85%, 90%, 95%, 97%, 98%, 99%, 99.5%, 99.9% or 100% identical to SEQ ID NO: 17, wherein the antibody or antigen binding fragment thereof comprises three sequential CDRs comprising or consisting of amino acid sequences of SEQ ID NOs: 36, 37, and 38. In some embodiments, any differences in sequence are limited to framework regions.

In one embodiment, the antibody or antigen binding fragment thereof that binds to DPEP-1 comprises an amino acid sequence that is at least 75%, 80%, 85%, 90%, 95%, 97%, 98%, 99%, 99.5%, 99.9% or 100% identical to SEQ ID NO: 18. In one embodiment, the antibody or antigen binding fragment thereof comprises the amino acid sequence of SEQ ID NO: 18. In one embodiment, the antibody or antigen binding fragment thereof that binds to DPEP-1 is an sdAB comprising at least one CDR having an amino acid sequence as set forth in any one of SEQ ID NOs: 39, 40, and 41. In one embodiment, the antibody or antigen binding fragment thereof that binds to DPEP-1 comprises an amino acid sequence that is at least 75%, 80%, 85%, 90%, 95%, 97%, 98%, 99%, 99.5%, 99.9% or 100% identical to SEQ ID NO: 18, wherein the antibody or antigen binding fragment thereof comprises three sequential CDRs comprising or consisting of amino acid sequences of SEQ ID NOs: 39, 40, and 41. In some embodiments, any differences in sequence are limited to framework regions.

In one embodiment, the antibody or antigen binding fragment thereof that binds to DPEP-1 comprises an amino acid sequence that is at least 75%, 80%, 85%, 90%, 95%, 97%, 98%, 99%, 99.5%, 99.9% or 100% identical to SEQ ID NO: 19. In one embodiment, the antibody or antigen binding fragment thereof comprises the amino acid sequence of SEQ ID NO: 19. In one embodiment, the antibody or antigen binding fragment thereof that binds to DPEP-1 is an sdAB comprising at least one CDR having an amino acid sequence as set forth in any one of SEQ ID NOs: 42, 43, and 44. In one embodiment, the antibody or antigen binding fragment thereof that binds to DPEP-1 comprises an amino acid sequence that is at least 75%, 80%, 85%, 90%, 95%, 97%, 98%, 99%, 99.5%, 99.9% or 100% identical to SEQ ID NO: 19, wherein the antibody or antigen binding fragment thereof comprises three sequential CDRs comprising or consisting of amino acid sequences of SEQ ID NOs: 42, 43, and 44. In some embodiments, any differences in sequence are limited to framework regions.

In one embodiment, the antibody or antigen binding fragment thereof that binds to DPEP-1 comprises an amino acid sequence that is at least 75%, 80%, 85%, 90%, 95%, 97%, 98%, 99%, 99.5%, 99.9% or 100% identical to SEQ ID NO: 20. In one embodiment, the antibody or antigen binding fragment thereof comprises the amino acid sequence of SEQ ID NO: 20. In one embodiment, the antibody or antigen binding fragment thereof that binds to DPEP-1 is an sdAB comprising at least one CDR having an amino acid sequence as set forth in any one of SEQ ID NOs: 45, 46, and 47. In one embodiment, the antibody or antigen binding fragment thereof that binds to DPEP-1 comprises an amino acid sequence that is at least 75%, 80%, 85%, 90%, 95%, 97%, 98%, 99%, 99.5%, 99.9% or 100% identical to SEQ ID NO: 20, wherein the antibody or antigen binding fragment thereof comprises three sequential CDRs comprising or consisting of amino acid sequences of SEQ ID NOs: 45, 46, and 47. In some embodiments, any differences in sequence are limited to framework regions.

In some embodiments, the binding agent is a monoclonal, polyclonal, chimeric, humanized antibody, or antigen binding fragment thereof. In an embodiment, the binding agent is an antigen binding fragment fused to a Fc domain. In some embodiments, the binding agent is an antigen binding fragment, and wherein the antigen binding fragment is a Fv, scFv, Fab, Fab′, F(ab′)2, dsFv, ds-scFv, sdAB, dimer, minibody, diabody, or multimer antigen binding fragment. In some embodiments, the antigen binding fragment is sdAB. In some embodiments, the antibody or antigen binding fragment comprises one or more amino acids selected from the group consisting of D-amino acids, modified amino acids, amino acid analogs or combinations thereof. In some embodiments, the modified amino acids comprise a modification selected from the group consisting of methylation, amidation, acetylation, and/or substitution with other chemical groups. In some embodiments, the antibody or antigen binding fragment is modified by pegylation, acetylation, glycosylation, biotinylation, or prenylation. In some embodiments, the antibody or antigen binding fragment is human, mouse, llama, rabbit, sheep, or goat antibody or antigen binding fragment thereof.

In some embodiments, the antibody or antigen binding fragment thereof that binds to DPEP-1 comprises an amino acid sequence that is at least 75%, 80%, 85%, 90%, 95%, 97%, 98%, 99%, 99.5%, 99.9% or 100% identical to any one of SEQ ID NOs: 12-20 and 48-56, wherein the antibody or antigen binding fragment is substituted with one or more D-amino or L-amino acids. In other embodiments, the antibody or antigen binding fragment thereof that binds to DPEP-1 provided herein can have 1, 2, 3, 4, or 5 amino acid residues removed from the N-terminus and/or C-terminus.

In one embodiment, the antibody or antigen binding fragment thereof that binds to DPEP-1 comprises an amino acid sequence that is at least 75%, 80%, 85%, 90%, 95%, 97%, 98%, 99%, 99.5%, 99.9% or 100% identical to any one of SEQ ID NOs: 1-9. In one embodiment, the antibody or antigen binding fragment thereof comprises the amino acid sequence of any one of SEQ ID NOs: 1-9.

In some embodiments, the antibody or antigen binding fragment thereof that binds to DPEP-1 comprises a Biotinylation Acceptor Peptide (BAP) and/or a His6 tag. In some embodiments, the BAP comprises the amino acid sequence of GLNDIFEAQKIEWHE (SEQ ID NO: 10). In some embodiments, the His6 tag comprises the amino sequence of HHHHHH (SEQ ID NO: 11). In some embodiments, the antibody or antigen binding fragment thereof that binds to DPEP-1 comprises the BAP and the His6 tag. In some embodiments, the antibody or antigen binding fragment thereof that binds to DPEP-1 comprises amino sequence of SEQ ID NOs: 10 and 11. In some embodiments, the BAP and the His6 tag is linked by a spacer having the amino acid sequence of LE. In some embodiments, the BAP and the His6 tag are the C-terminus of the antibody or antigen binding fragment thereof.

### B. Modified Antibodies and Antibody Analogs

In various embodiments, the antibody or antigen binding fragment thereof comprises amino acids, including carboxy- and/or amino-terminal amino acids in antibodies, or can be modified by PEGylation, methylation, amidation, acetylation, prenylation and/or substitution with other chemical groups that can change the antibody's or antigen binding fragment's circulating half-life without adversely affecting its activity. Examples of unconventional or un-natural amino acids include, but not limited to, citrulline, norleucine, ornithine, norvaline, 4-(E)-butenyl-4(R)-methyl-N-methylthreonine (MeBmt), N-methyl-leucine (MeLeu), aminoisobutyric acid, statine, and N-methyl-alanine (MeAla). Amino acids may participate in a disulfide bond. In certain embodiments, the amino acid has the general structure H₂N—C(H)(R)—COOH. In certain embodiments, the amino acid is a naturally-occurring amino acid. In certain embodiments, the amino acid is a synthetic or un-natural amino acid (e.g., α,α-disubstituted amino acids, N-alkyl amino acids); in some embodiments, the amino acid is a D-amino acid; in certain embodiments, the amino acid is an L-amino acid.

The fragment crystallizable region (Fc region or Fc domain) is the tail region of an antibody that interacts with cell surface receptors called Fc receptors. This property allows antibodies to activate the immune system. In IgG, IgA and IgD antibody isotypes, the Fc domain is composed of two identical protein fragments, derived from the second and third constant domains of the antibody's two heavy chains. In IgM and IgE, their Fc domains contain three heavy chain constant domains (CH domains 2-4) in each polypeptide chain. The antibody or antigen binding fragment thereof described herein can be fused to Fc domain and can also be a multimer, for example, to generate bispecific/biparatopic molecules. In some embodiments, the antibody or antigen binding fragment thereof that binds to DPEP-1 is fused to a Fc domain. In some embodiments, the antibody or antigen binding fragment thereof that binds to DPEP-1 is a multimer. In some embodiments, the antibody or antigen binding fragment thereof that binds to DPEP-1 is fused to a Fc domain and is a multimer. In some embodiments, the Fc domain is from IgG, IgA, IgD, IgM, or IgE. In some embodiments, the Fc domain is a human Fc domain. In some embodiments, the Fc domain is from human IgG1, IgG2, IgG3, or lgG4.

Also provided is a fusion protein comprising the antibody or antigen binding fragment thereof that binds to DPEP-1 and a Fc domain. In some embodiments, the fusion protein is a multimer. In some embodiments, the binding agent is a fusion protein of an antigen binding fragment that binds to DPEP-1 and a Fc domain. In some embodiments, the fusion protein comprises an amino acid sequence that is at least 75%, 80%, 85%, 90%, 95%, 97%, 98%, 99%, 99.5%, 99.9% or 100% identical to any one of SEQ ID NOs: 12-20 and a Fc domain. In some embodiments, the fusion protein comprises the amino acid sequence of any one of SEQ ID NOs: 12-20 and a Fc domain. In some embodiments, the fusion protein comprises an amino acid sequence that is at least 75%, 80%, 85%, 90%, 95%, 97%, 98%, 99%, 99.5%, 99.9% or 100% identical to any one of SEQ ID NOs: 48-56. In some embodiments, the fusion protein comprises the amino acid sequence of any one of SEQ ID NOs: 48-56.

In one embodiment, the fusion protein comprises an amino acid sequence that is at least 75%, 80%, 85%, 90%, 95%, 97%, 98%, 99%, 99.5%, 99.9% or 100% identical to SEQ ID NO: 12 and a Fc domain. In one embodiment, the fusion protein comprises an amino acid sequence that is at least 75%, 80%, 85%, 90%, 95%, 97%, 98%, 99%, 99.5%, 99.9% or 100% identical to SEQ ID NO: 48. In one embodiment, the fusion protein comprises the amino acid sequence of SEQ ID NO: 48. In one embodiment, the fusion protein comprising at least one CDR having an amino acid sequence as set forth in any one of SEQ ID NOs: 21, 22, and 23, and a Fc domain. In one embodiment, the fusion protein comprises an amino acid sequence that is at least 75%, 80%, 85%, 90%, 95%, 97%, 98%, 99%, 99.5%, 99.9% or 100% identical to SEQ ID NO: 12 and a Fc domain, wherein the fusion protein comprises three sequential CDRs comprising or consisting of amino acid sequences of SEQ ID NOs: 21, 22, and 23. In one embodiment, the fusion protein comprises the amino acid sequence of SEQ ID NO: 12 and a Fc domain. In one embodiment, the fusion protein comprises an amino acid sequence that is at least 75%, 80%, 85%, 90%, 95%, 97%, 98%, 99%, 99.5%, 99.9% or 100% identical to SEQ ID NO: 48, wherein the fusion protein comprises three sequential CDRs comprising or consisting of amino acid sequences of SEQ ID NOs: 21, 22, and 23. In some embodiments, the fusion protein comprises the amino acid sequence of SEQ ID NO: 48. In some embodiments, any differences in sequence are limited to framework regions or the Fc domain.

In one embodiment, the fusion protein comprises an amino acid sequence that is at least 75%, 80%, 85%, 90%, 95%, 97%, 98%, 99%, 99.5%, 99.9% or 100% identical to SEQ ID NO: 13 and a Fc domain. In one embodiment, the fusion protein comprises an amino acid sequence that is at least 75%, 80%, 85%, 90%, 95%, 97%, 98%, 99%, 99.5%, 99.9% or 100% identical to SEQ ID NO: 49. In one embodiment, the fusion protein comprises the amino acid sequence of SEQ ID NO: 49. In one embodiment, the fusion protein comprising at least one CDR having an amino acid sequence as set forth in any one of SEQ ID NOs: 24, 25, and 26, and a Fc domain. In one embodiment, the fusion protein comprises an amino acid sequence that is at least 75%, 80%, 85%, 90%, 95%, 97%, 98%, 99%, 99.5%, 99.9% or 100% identical to SEQ ID NO: 13 and a Fc domain, wherein the fusion protein comprises three sequential CDRs comprising or consisting of amino acid sequences of SEQ ID NOs: 24, 25, and 26. In one embodiment, the fusion protein comprises the amino acid sequence of SEQ ID NO: 13 and a Fc domain. In one embodiment, the fusion protein comprises an amino acid sequence that is at least 75%, 80%, 85%, 90%, 95%, 97%, 98%, 99%, 99.5%, 99.9% or 100% identical to SEQ ID NO: 49, wherein the fusion protein comprises three sequential CDRs comprising or consisting of amino acid sequences of SEQ ID NOs: 24, 25, and 26. In some embodiments, the fusion protein comprises the amino acid sequence of SEQ ID NO: 49. In some embodiments, any differences in sequence are limited to framework regions or the Fc domain.

In one embodiment, the fusion protein comprises an amino acid sequence that is at least 75%, 80%, 85%, 90%, 95%, 97%, 98%, 99%, 99.5%, 99.9% or 100% identical to SEQ ID NO: 14 and a Fc domain. In one embodiment, the fusion protein comprises an amino acid sequence that is at least 75%, 80%, 85%, 90%, 95%, 97%, 98%, 99%, 99.5%, 99.9% or 100% identical to SEQ ID NO: 50. In one embodiment, the fusion protein comprises the amino acid sequence of SEQ ID NO: 50. In one embodiment, the fusion protein comprising at least one CDR having an amino acid sequence as set forth in any one of SEQ ID NOs: 27, 28, and 29 and a Fc domain. In one embodiment, the fusion protein comprises an amino acid sequence that is at least 75%, 80%, 85%, 90%, 95%, 97%, 98%, 99%, 99.5%, 99.9% or 100% identical to SEQ ID NO: 14 and a Fc domain, wherein the fusion protein comprises three sequential CDRs comprising or consisting of amino acid sequences of SEQ ID NOs: 27, 28, and 29. In one embodiment, the fusion protein comprises the amino acid sequence of SEQ ID NO: 14 and a Fc domain. In one embodiment, the fusion protein comprises an amino acid sequence that is at least 75%, 80%, 85%, 90%, 95%, 97%, 98%, 99%, 99.5%, 99.9% or 100% identical to SEQ ID NO: 50, wherein the fusion protein comprises three sequential CDRs comprising or consisting of amino acid sequences of SEQ ID NOs: 27, 28, and 29. In some embodiments, the fusion protein comprises the amino acid sequence of SEQ ID NO: 50. In some embodiments, any differences in sequence are limited to framework regions or the Fc domain.

In one embodiment, the fusion protein comprises an amino acid sequence that is at least 75%, 80%, 85%, 90%, 95%, 97%, 98%, 99%, 99.5%, 99.9% or 100% identical to SEQ ID NO: 15 and a Fc domain. In one embodiment, the fusion protein comprises an amino acid sequence that is at least 75%, 80%, 85%, 90%, 95%, 97%, 98%, 99%, 99.5%, 99.9% or 100% identical to SEQ ID NO: 51. In one embodiment, the fusion protein comprises the amino acid sequence of SEQ ID NO: 51. In one embodiment, the fusion protein comprising at least one CDR having an amino acid sequence as set forth in any one of SEQ ID NOs: 30, 31, and 32, and a Fc domain. In one embodiment, the fusion protein comprises an amino acid sequence that is at least 75%, 80%, 85%, 90%, 95%, 97%, 98%, 99%, 99.5%, 99.9% or 100% identical to SEQ ID NO: 15 and a Fc domain, wherein the fusion protein comprises three sequential CDRs comprising or consisting of amino acid sequences of SEQ ID NOs: 30, 31, and 32. In one embodiment, the fusion protein comprises the amino acid sequence of SEQ ID NO: 15 and a Fc domain. In one embodiment, the fusion protein comprises an amino acid sequence that is at least 75%, 80%, 85%, 90%, 95%, 97%, 98%, 99%, 99.5%, 99.9% or 100% identical to SEQ ID NO: 51, wherein the fusion protein comprises three sequential CDRs comprising or consisting of amino acid sequences of SEQ ID NOs: 30, 31, and 32. In some embodiments, the fusion protein comprises the amino acid sequence of SEQ ID NO: 51. In some embodiments, any differences in sequence are limited to framework regions or the Fc domain.

In one embodiment, the fusion protein comprises an amino acid sequence that is at least 75%, 80%, 85%, 90%, 95%, 97%, 98%, 99%, 99.5%, 99.9% or 100% identical to SEQ ID NO: 16 and a Fc domain. In one embodiment, the fusion protein comprises an amino acid sequence that is at least 75%, 80%, 85%, 90%, 95%, 97%, 98%, 99%, 99.5%, 99.9% or 100% identical to SEQ ID NO: 52. In one embodiment, the fusion protein comprises the amino acid sequence of SEQ ID NO: 52. In one embodiment, the fusion protein comprising at least one CDR having an amino acid sequence as set forth in any one of SEQ ID NOs: 33, 34, and 35, and a Fc domain. In one embodiment, the fusion protein comprises an amino acid sequence that is at least 75%, 80%, 85%, 90%, 95%, 97%, 98%, 99%, 99.5%, 99.9% or 100% identical to SEQ ID NO: 16 and a Fc domain, wherein the fusion protein comprises three sequential CDRs comprising or consisting of amino acid sequences of SEQ ID NOs: 33, 34, and 35. In one embodiment, the fusion protein comprises the amino acid sequence of SEQ ID NO: 16 and a Fc domain. In one embodiment, the fusion protein comprises an amino acid sequence that is at least 75%, 80%, 85%, 90%, 95%, 97%, 98%, 99%, 99.5%, 99.9% or 100% identical to SEQ ID NO: 52, wherein the fusion protein comprises three sequential CDRs comprising or consisting of amino acid sequences of SEQ ID NOs: 33, 34, and 35. In some embodiments, the fusion protein comprises the amino acid sequence of SEQ ID NO: 52. In some embodiments, any differences in sequence are limited to framework regions or the Fc domain.

In one embodiment, the fusion protein comprises an amino acid sequence that is at least 75%, 80%, 85%, 90%, 95%, 97%, 98%, 99%, 99.5%, 99.9% or 100% identical to SEQ ID NO: 17 and a Fc domain. In one embodiment, the fusion protein comprises an amino acid sequence that is at least 75%, 80%, 85%, 90%, 95%, 97%, 98%, 99%, 99.5%, 99.9% or 100% identical to SEQ ID NO: 53. In one embodiment, the fusion protein comprises the amino acid sequence of SEQ ID NO: 53. In one embodiment, the fusion protein comprising at least one CDR having an amino acid sequence as set forth in any one of SEQ ID NOs: 36, 37, and 38, and a Fc domain. In one embodiment, the fusion protein comprises an amino acid sequence that is at least 75%, 80%, 85%, 90%, 95%, 97%, 98%, 99%, 99.5%, 99.9% or 100% identical to SEQ ID NO: 17 and a Fc domain, wherein the fusion protein comprises three sequential CDRs comprising or consisting of amino acid sequences of SEQ ID NOs: 36, 37, and 38. In one embodiment, the fusion protein comprises the amino acid sequence of SEQ ID NO: 17 and a Fc domain. In one embodiment, the fusion protein comprises an amino acid sequence that is at least 75%, 80%, 85%, 90%, 95%, 97%, 98%, 99%, 99.5%, 99.9% or 100% identical to SEQ ID NO: 53, wherein the fusion protein comprises comprising three sequential CDRs comprising or consisting of amino acid sequences of SEQ ID NOs: 36, 37, and 38. In some embodiments, the fusion protein comprises the amino acid sequence of SEQ ID NO: 53. In some embodiments, any differences in sequence are limited to framework regions or the Fc domain.

In one embodiment, the fusion protein comprises an amino acid sequence that is at least 75%, 80%, 85%, 90%, 95%, 97%, 98%, 99%, 99.5%, 99.9% or 100% identical to SEQ ID NO: 18 and a Fc domain. In one embodiment, the fusion protein comprises an amino acid sequence that is at least 75%, 80%, 85%, 90%, 95%, 97%, 98%, 99%, 99.5%, 99.9% or 100% identical to SEQ ID NO: 54. In one embodiment, the fusion protein comprises the amino acid sequence of SEQ ID NO: 54. In one embodiment, the fusion protein comprising at least one CDR having an amino acid sequence as set forth in any one of SEQ ID NOs: 39, 40, and 41, and a Fc domain. In one embodiment, the fusion protein comprises an amino acid sequence that is at least 75%, 80%, 85%, 90%, 95%, 97%, 98%, 99%, 99.5%, 99.9% or 100% identical to SEQ ID NO: 18 and a Fc domain, wherein the fusion protein comprises three sequential CDRs comprising or consisting of amino acid sequences of SEQ ID NOs: 39, 40, and 41. In one embodiment, the fusion protein comprises the amino acid sequence of SEQ ID NO: 18 and a Fc domain. In one embodiment, the fusion protein comprises an amino acid sequence that is at least 75%, 80%, 85%, 90%, 95%, 97%, 98%, 99%, 99.5%, 99.9% or 100% identical to SEQ ID NO: 54, wherein the fusion protein comprises three sequential CDRs comprising or consisting of amino acid sequences of SEQ ID NOs: 39, 40, and 41. In some embodiments, the fusion protein comprises the amino acid sequence of SEQ ID NO: 54. In some embodiments, any differences in sequence are limited to framework regions or the Fc domain.

In one embodiment, the fusion protein comprises an amino acid sequence that is at least 75%, 80%, 85%, 90%, 95%, 97%, 98%, 99%, 99.5%, 99.9% or 100% identical to SEQ ID NO: 19 and a Fc domain. In one embodiment, the fusion protein comprises an amino acid sequence that is at least 75%, 80%, 85%, 90%, 95%, 97%, 98%, 99%, 99.5%, 99.9% or 100% identical to SEQ ID NO: 55. In one embodiment, the fusion protein comprises the amino acid sequence of SEQ ID NO: 55 In one embodiment, the fusion protein comprising at least one CDR having an amino acid sequence as set forth in any one of SEQ ID NOs: 42, 43, and 44, and a Fc domain. In one embodiment, the fusion protein comprises an amino acid sequence that is at least 75%, 80%, 85%, 90%, 95%, 97%, 98%, 99%, 99.5%, 99.9% or 100% identical to SEQ ID NO: 19 and a Fc domain, wherein the fusion protein comprises three sequential CDRs comprising or consisting of amino acid sequences of SEQ ID NOs: 42, 43, and 44. In one embodiment, the fusion protein comprises the amino acid sequence of SEQ ID NO: 19 and a Fc domain. In one embodiment, the fusion protein comprises an amino acid sequence that is at least 75%, 80%, 85%, 90%, 95%, 97%, 98%, 99%, 99.5%, 99.9% or 100% identical to SEQ ID NO: 55, wherein the fusion protein comprises three sequential CDRs comprising or consisting of amino acid sequences of SEQ ID NOs: 42, 43, and 44. In some embodiments, the fusion protein comprises the amino acid sequence of SEQ ID NO: 55. In some embodiments, any differences in sequence are limited to framework regions or the Fc domain.

In one embodiment, the fusion protein comprises an amino acid sequence that is at least 75%, 80%, 85%, 90%, 95%, 97%, 98%, 99%, 99.5%, 99.9% or 100% identical to SEQ ID NO: 20 and a Fc domain. In one embodiment, the fusion protein comprises an amino acid sequence that is at least 75%, 80%, 85%, 90%, 95%, 97%, 98%, 99%, 99.5%, 99.9% or 100% identical to SEQ ID NO: 56. In one embodiment, the fusion protein comprises the amino acid sequence of SEQ ID NO: 56. In one embodiment, the fusion protein comprising at least one CDR having an amino acid sequence as set forth in any one of SEQ ID NOs: 45, 46, and 47, and a Fc domain. In one embodiment, the fusion protein comprises an amino acid sequence that is at least 75%, 80%, 85%, 90%, 95%, 97%, 98%, 99%, 99.5%, 99.9% or 100% identical to SEQ ID NO: 20 and a Fc domain, wherein the fusion protein comprises three sequential CDRs comprising or consisting of amino acid sequences of SEQ ID NOs: 45, 56, and 47. In one embodiment, the fusion protein comprises the amino acid sequence of SEQ ID NO: 20 and a Fc domain. In one embodiment, the fusion protein comprises an amino acid sequence that is at least 75%, 80%, 85%, 90%, 95%, 97%, 98%, 99%, 99.5%, 99.9% or 100% identical to SEQ ID NO: 56, wherein the fusion protein comprises three sequential CDRs comprising or consisting of amino acid sequences of SEQ ID NOs: 45, 46, and 47. In some embodiments, the fusion protein comprises the amino acid sequence of SEQ ID NO: 56. In some embodiments, any differences in sequence are limited to framework regions or the Fc domain.

### B. Rational Design and Structure-Function Analysis of DPEP-1 Binding Antibodies

Different techniques give different and complementary information about protein structure. The primary structure is obtained by biochemical methods, either by direct determination of the amino acid sequence from the protein, or from the nucleotide sequence of the corresponding gene or cDNA. The quaternary structure of large proteins or aggregates can also be determined by electron microscopy. To obtain the secondary and tertiary structure, which requires detailed information about the arrangement of atoms within a protein, x-ray crystallography is commonly used. Other structural technologies to assess antibody or fragment structure and function include hydrogen-deuterium exchange mass spectrometry, bio-NMR, and cryo-EM.

The first prerequisite for solving the three-dimensional structure of a protein by x-ray crystallography is a well-ordered crystal that will diffract x-rays strongly. The crystallographic method directs a beam of x-rays onto a regular, repeating array of many identical molecules so that the x-rays are diffracted from it in a pattern from which the structure of an individual molecule can be retrieved. Well-ordered crystals of globular protein molecules are large, spherical, or ellipsoidal objects with irregular surfaces, and crystals thereof contain large holes or channels that are formed between the individual molecules. These channels, which usually occupy more than half the volume of the crystal, are filled with disordered solvent molecules. The protein molecules are in contact with each other at only a few small regions. This is one reason why structures of proteins determined by x-ray crystallography are generally the same as those for the proteins in solution

X-ray crystallography can be used to screen compounds that are not known ligands of a target biomolecule for their ability to bind the target biomolecule. The method includes obtaining a crystal of a target biomolecule; exposing the target biomolecule crystal to one or more test samples; and obtaining an X-ray crystal diffraction pattern to determine whether a ligand/receptor complex is formed.

The DPEP-1 receptor can be exposed to the test antibodies by either co-crystallizing a biomolecule in the presence of one or more test samples or soaking the biomolecule crystal in a solution of one or more test samples. In another embodiment, structural information from ligand/receptor complexes are used to design ligands that bind tighter, that bind more specifically, that have better biological activity or that have better safety profile. These may include small molecules or other biotherapeutics such as antibodies.

Antibodies described herein can be fully characterized using mass spectrometry, high performance liquid chromatography (HPLC) and amino acid analysis (AAA).

Mass spectrometry is used to obtain distance constraints between amino acid residues of a protein to be used in determining the structure the protein.

While not to be bound by any particular mechanism, it is believed that the antibody or fragment structure and any modifications that stabilize the tertiary structure enhance binding to DPEP-1.

In one embodiment, determining key DPEP-1 epitopes that bind DPEP-1 antibody or antigen binding fragment can also be used to develop therapeutic antibodies targeting DPEP-1, leukocyte adhesion and cancer metastasis. Examples of such methods include but not limited to crystallizing DPEP-1 bound to DPEP-1 antibody or antigen binding fragment and analysis by X-ray diffraction. In another embodiment, photochemical crosslinking of DPEP-1 antibody or antigen binding fragment bound to DPEP-1 followed by protease digestion and mass spectrometry (Ngai et al, J Biol Chem, 269(3), 2165-2172 (1994)) is used to identify the key DPEP-1 antibody or antigen binding fragment amino acids and DPEP-1 domains involved in the interaction. In various embodiments, in silico modeling is performed to develop novel pharmaceutical compositions.

Unless defined otherwise, the scientific and technological terms and nomenclature used herein have the same meaning as commonly understood by a person of ordinary skill to which this disclosure pertains. Generally, the procedures of cell cultures, infection, molecular biology methods and the like are common methods used in the art. Such standard techniques can be found in reference manuals such as, for example, Ausubel et al., Current Protocols in Molecular Biology, Wiley Interscience, New York, 2001; and Sambrook et al., Molecular Cloning: A Laboratory Manual, 3rd edition, Cold Spring Harbor Laboratory Press, N.Y., 2001; herein incorporated by reference.

### IV. Pharmaceutical Formulations and Medicaments

In another aspect, the compounds or agents described herein, as well as variants and modifications thereof, are provided as a pharmaceutical composition for therapeutic use. In one embodiment, the pharmaceutical formulation comprises an isolated antibody or antigen binding fragment includes a sequence listed in Table 1. In another embodiment, the pharmaceutical formulation comprises an isolated antibody or antigen binding fragment contained as an insert in a phage virus, and/or may further comprise 1, 2, 3, 4, 5 additional amino acid residues at the N-terminus and/or C-terminus of the antibody or antigen binding fragment sequence.

Representative delivery regimens include oral, parenteral (including subcutaneous, intramuscular and intravenous injection), rectal, buccal (including sublingual), transdermal, inhalation, ocular and intranasal. In one embodiment, delivery of compounds entails subcutaneous injection of a controlled-release injectable formulation. In some embodiments, compounds described herein are useful for subcutaneous, intranasal and inhalation administration.

The selection of the exact dose and composition and the most appropriate delivery regimen will be influenced by, inter alia, the pharmacological properties of the selected antibody, the nature and severity of the condition being treated, and the physical condition and mental acuity of the recipient. Additionally, the route of administration will result in differential amounts of absorbed material. Bioavailabilities for administration of compounds through different routes are particularly variable, with amounts from less than 1% to near 100% being seen. Typically, bioavailability from routes other than intravenous, intraperitoneal or subcutaneous injection are 50% or less.

The pharmaceutical compositions or formulations of the present disclosure can be formulated with a physiologically acceptable carrier or excipient to prepare a pharmaceutical composition. The carrier and composition can be sterile. The formulation should suit the mode of administration, for example intravenous or subcutaneous administration. Methods of formulating compositions are known in the art (see, e.g., Remington's Pharmaceuticals Sciences, 17th Edition, Mack Publishing Co., (Alfonso R. Gennaro, editor) (1989); herein incorporated by reference).

Suitable pharmaceutically acceptable carriers include, but not limited to, water, salt solutions (e.g., NaCl), saline, buffered saline, alcohols, glycerol, ethanol, gum arabic, vegetable oils, benzyl alcohols, polyethylene glycols, gelatin, carbohydrates such as lactose, amylose or starch, sugars such as mannitol, sucrose, or others, dextrose, magnesium stearate, talc, silicic acid, viscous paraffin, perfume oil, fatty acid esters, hydroxymethylcellulose, polyvinyl pyrolidone, etc., as well as combinations thereof. The pharmaceutical preparations can, if desired, be mixed with auxiliary agents (e.g., lubricants, preservatives, stabilizers, wetting agents, emulsifiers, salts for influencing osmotic pressure, buffers, coloring and/or aromatic substances and the like) which do not deleteriously react with the active compounds or interference with their activity. In an embodiment, a water-soluble carrier suitable for intravenous administration is used. Pharmaceutically acceptable salts retain the desired biological activity of the parent antibody or antigen binding fragment thereof without toxic side effects.

The composition or medicament, if desired, can also contain minor amounts of wetting or emulsifying agents, or pH buffering agents. The composition can be a liquid solution, suspension, emulsion, sustained release formulation, or powder. The composition can also be formulated as a suppository, with traditional binders and carriers such as triglycerides.

The composition or medicament can be formulated in accordance with the routine procedures as a pharmaceutical composition adapted for administration to human beings. For example, in an embodiment, a composition for intravenous administration typically is a solution in sterile isotonic aqueous buffer. Where necessary, the composition may also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachette indicating the quantity of active agent. Where the composition is to be administered by infusion, it can be dispensed with an infusion bottle containing sterile pharmaceutical grade water, saline or dextrose/water. Where the composition is administered by injection, an ampule of sterile water for injection or saline can be provided so that the ingredients may be mixed prior to administration.

In some embodiments, the pharmaceutical composition comprise a liquid carrier such as, but not limited to, water, saline, phosphate buffered saline, Ringer's solution, dextrose solution, serum-containing solutions, Hank's solution, other aqueous physiologically balanced solutions, oils, esters and glycols.

The compounds, including antibodies or antigen binding fragments, as described herein can be formulated as neutral or salt forms. As stated above, pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The pharmaceutical formulations of the present disclosure contain, as the binding agent, antibody or antigen binding fragment may be mixed with an excipient, diluted by an excipient or enclosed within a carrier, which can be in the form of a capsule, sachet, paper or other container, according to well-known methods and pharmaceutical compositions. The composition may be administered by any route suitable for binding agent, antibody or antigen binding fragment administration, including parenteral, intravenous, subcutaneous, or intramuscular administration. Typically, the binding agent, antibody or antigen binding fragment is dissolved or suspended in a sterile injectable solution, at a concentration sufficient to provide the required dose in 0.5 to 2 ml or less. Pharmaceutical compositions of this disclosure suitable for parenteral administrations comprise one or more compounds of the disclosure in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, solutes which render the formulation isotonic with the blood of the intended recipient or suspending or thickening agents.

Injectable depot forms are made by forming microencapsulated matrices of the drug in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues. The injectable materials can be sterilized for example, by filtration through a bacterial-retaining filter.

The pharmaceutical compositions may be presented in unit-dose or multi-dose sealed containers, for example, ampules and vials, and may be stored in a lyophilized condition requiring only the addition of the sterile liquid carrier, for example water for injection, immediately prior to use. Extemporaneous injection solutions and suspensions may be prepared from sterile powders, granules and tablets of the type described above.

### V. Methods of Treatment and Uses

Methods of treatment and uses are contemplated for diseases and conditions associated with inflammation including particularly diseases and conditions where inflammation is caused by ischemia/reperfusion injury to a tissue or organ. Ischemia followed by reperfusion in an organ produces structural and functional abnormalities in the tissue of that organ and others. Neutrophil infiltration, hemorrhage, edema and necrosis are all observed in tissues following an ischemia/reperfusion injury. The DPEP-1 target represents a previously undescribed pathway for inflammation which opens up the opportunity for dipeptidase inhibitors such as those described herein to be used to treat or prevent diseases and conditions mediated by inflammation.

Accordingly, in one embodiment there is provided, a method of treating or preventing an inflammatory disorder in a human subject in need thereof, comprising administering to the subject an effective amount of a binding agent or pharmaceutical composition of the present disclosure. In another embodiment, there is provided a use of a binding agent or pharmaceutical composition of the present disclosure for treating or preventing an inflammatory disorder in a human subject in need thereof. In a further embodiment, there is provided a use of binding agent or pharmaceutical composition of the present disclosure for the manufacture of a medicament for treating or preventing an inflammatory disorder in a human subject in need thereof. In a further embodiment, there is provided a binding agent or pharmaceutical composition of the present disclosure for use in treating or preventing an inflammatory disorder in a human subject in need thereof.

A non-limiting list of common diseases and medical problems that are directly associated with inflammation include: arthritis, kidney failure, lupus, asthma, psoriasis, pancreatitis, allergy, fibrosis, surgical complications, anemia, fibromyalgia. Other diseases associated with chronic inflammation include cancer, which is caused by chronic inflammation; heart attack where chronic inflammation contributes to coronary atherosclerosis; Alzheimer's disease where chronic inflammation destroys brain cells; congestive heart failure where chronic inflammation causes heart muscle wasting; stroke where chronic inflammation promotes thrombo-embolic events; and aortic valve stenosis where chronic inflammation damages heart valves. Arteriosclerosis, osteoporosis, Parkinson's disease, infection (sepsis), inflammatory bowel disease including Crohn's disease and ulcerative colitis as well as multiple sclerosis.

In particular embodiments, the methods or uses described herein are useful for protecting tissues and organs from damage associated with conditions such as, but not limited to sepsis-induced injury such as bacterial or viral sepsis-induced injury, acute organ injury (for example acute kidney injury in the setting of low blood pressure).

In other embodiments, the methods or uses described herein are useful for protecting tissues and organs from damage associated with sepsis-induced conditions such as bacterial or viral sepsis-induced conditions, acute respiratory distress syndrome, encephalopathy, sepsis-induced liver failure, sepsis-induced kidney failure or sepsis-induced heart failure. Coronavirus disease 2019 (COVID-19) is a contagious disease caused by severe acute respiratory syndrome coronavirus 2 (SARS-CoV-2), which can lead to complications such as sepsis resulting in a COVID-19 sepsis-induced condition. Accordingly, in some embodiments, the viral sepsis-induced condition comprises a COVID-19 sepsis-induced condition.

In other embodiments, the methods or uses described herein are useful for protecting tissues and organs from damage associated with ischemia-reperfusion injury such as, but not limited to peri-operative procedures, heart failure, liver failure, stroke, myocardial infarct, shock liver, spinal cord injury, brain injury, and the like. These binding agent, antibody or antigen binding fragment thereof or composition can also be used to prevent or treat ischemia-reperfusion injury in high risk patients.

In other embodiments, the methods and uses are also useful prior to angioplasty or thrombolytic therapy, or after transplantation or reperfusion of an ischemic organ following surgery, angioplasty or thrombolytic therapy.

Other examples of surgical procedures and organs at risk of ischemia reperfusion injury during these procedures include, but not limited, brain injury during carotid artery surgery, cerebral vascular surgery and surgery of the heart and aorta; acute kidney injury during cardiac surgery or major abdominal or thoracic surgery; lung injury following thromboembolectomy or the use of cardiopulmonary bypass during lung and heart surgery; heart injury following revascularization (coronary artery bypass graft surgery); intestinal injury following surgery on the mesenteric arteries; and skin injury following harvesting of a skin graft.

Additional surgical procedures for which this method or use of binding agents, antibodies, antigen binding fragments, or compositions of the present disclosure is useful include harvesting donor organs for transplantation. In other embodiments, the methods or uses are also useful for the protection of allograft organs during donor procurement, ex vivo handling and implantation into a transplant recipient. Binding agents, antibodies, antigen binding fragments, or compositions of the present disclosure can be administered or used prior to, during or following harvesting a donor organ which will be transplanted, prior to or during a surgical procedure in which ischemia is expected.

Hence, the disclosure relates to a method for preventing, limiting, or treating ischemia reperfusion injury in a subject, comprising the steps of identifying a subject that has undergone an ischemic event, or in which an ischemic event is imminent or is at risk for having an ischemic event and administering a therapeutically effective or prophylactically effective amount of the binding agents, antibodies, antigen binding fragments, or compositions described herein. The disclosure also relates to a use of a binding agent, antibody or antigen binding fragment thereof or composition described herein for preventing, limiting, or treating ischemia reperfusion injury in a subject. In some embodiments, the subject has undergone an ischemic event, an ischemic event is imminent in the subject, or the subject is at risk for having an ischemic event. The disclosure also relates to a use of a binding agent, antibody or antigen binding fragment thereof or composition described herein for the manufacture of a medicament for preventing, limiting, or treating ischemia reperfusion injury in a subject. In some embodiments, the subject has undergone an ischemic event, an ischemic event is imminent in the subject, or the subject is at risk for having an ischemic event. The disclosure further relates to a binding agent, antibody or antigen binding fragment thereof or composition described herein for use in preventing, limiting, or treating ischemia reperfusion injury in a subject. In some embodiments, the subject has undergone an ischemic event, an ischemic event is imminent in the subject, or the subject is at risk for having an ischemic event.

In one embodiment, a method or use is disclosed for extracting an organ from a donor, comprising administering or using a binding agent, antibody, antigen binding fragment thereof, or composition that binds to DPEP-1 disclosed herein to the donor prior to extraction of the organ. Optionally, a method or use is disclosed for the recipient of a transplant organ, comprising administering or using a binding agent, antibody, antigen binding fragment thereof, or composition that binds to DPEP-1 prior to organ implantation. The method or use may further comprise monitoring the level of one or more inflammatory makers to determine whether the one or more markers is below a designated level prior to extraction, i.e., to determine whether the organ meets a predetermined marker profile.

In a particular embodiment, the organ is selected from the group consisting of consisting of heart, liver, kidney, brain, intestine (large or small), pancreas, lung, stomach, bladder, spleen, ovaries, testes, skeletal muscle and combinations thereof.

In a particular embodiment, the donor is a marginal donor. In one embodiment, the marginal donor is selected from the group consisting of complex living donors, a non-heart beating donor (NHBD) or a deceased cardiac donor.

In a particular embodiment, the complex living donor is of advanced age, e.g., greater than about 60 years old, greater than about 65 years old or greater than about 70 years old.

In another particular embodiment, the complex living donor has one or more risk factors selected from the group consisting of obesity, hypertension, diabetes, nephrolithiasis (kidney stones), transmissible infectious disease (e.g., a viral infection), or combinations thereof.

In one embodiment, the method or use further comprises storing the organ. Optionally, the level of one or more inflammatory markers may be measured one or more time during storage of the organ.

In another embodiment, the method or use further comprising providing the organ to a recipient, e.g., by transplantation. Optionally, the level of one or more inflammatory markers may by measured after the organ is provided, e.g., during the immediate postreperfusion period.

The inflammatory markers can be, but not limited to, IL-12, IP-10, IL-1β, IL-5, GM-CSF, IFNγ or IL-1α. In one embodiment, the one or more inflammatory markers are selected from the group consisting of IL-12, IP-10, IL-1β, IL-5, GM-CSF, IFNγ or IL-1α.

Optionally, in some embodiments, one or more additional agents (e.g., antioxidant) may be administered to the donor or for use in the donor prior to extraction of the organ. In some embodiments, one or more additional agents (e.g., antioxidant) may be administered to the recipient or for use in the recipient prior to implantation of the organ. In some embodiments, the one or more additional agents may be administered or for use prior to, contemporaneously therewith or after administration of a binding agent, antibody, antigen binding fragment, or composition that binds to DPEP-1. In some embodiments, the additional agent may be, for example, a small molecule, biologic agent or therapeutic gas.

In certain embodiments, the method or use disclosed herein may result in one of more beneficial effects including, without limitation, improved graft function, reduced graft dysfunction, improve graft survival (including long term survival), reduced graft deterioration, reduced incidence of delayed graft function (DGF) or the like.

In one embodiment, the method or use results in an increase in graft survival compared to survival of grafts to which a binding agent, antibody, antigen binding fragment, or composition that binds to DPEP-1 is not administered or used prior to extraction or implantation. Graft survival may be measured, e.g., at six months, one year or three years following transplantation. In a particular embodiment, graft survival is increased by about 5%, about 10%, about 15%, about 20% or about 25% or more.

In one embodiment, a method or use of preserving an organ is disclosed, comprising exposing a stored organ (i.e., an extracted organ awaiting transplantation) to a binding agent, antibody, antigen binding fragment, or composition that binds to DPEP-1 disclosed herein. Optionally, the method or use further comprising monitoring the level of inflammatory markers one or more times to determine whether they fall below a designated level. In certain embodiments, the organ is stored in an organ transplant solution. In certain embodiments, the organ is stored at temperatures between about 0° C. and 4° C. In another embodiment, the organ is stored at a temperature of about 37° C., i.e., under non-thermic conditions. In certain embodiment, the stored organ is connected to or associated with an organ perfusion system.

In a particular embodiment, the method or use of preserving the organ disclosed herein permits an increase in the maximum cold ischemia time for the particular organ without impairment, e.g., without an increase in delayed graft function (DGF). In certain embodiments, the increase in between about 5 and about 50%, more particularly, between about 5 and about 25%, or about 5%, about 10%, about 15%, about 20%, about 25% or about 30% or more.

In another embodiment, a method of preventing ischemia-reperfusion related injury in an organ transplant patient is provided, comprising administering a binding agent, antibody, antigen binding fragment or composition that binds to DPEP-1 disclosed herein prior to, simultaneously with or after providing the organ to the patient. In another embodiment, a use of a binding agent, antibody or antigen binding fragment thereof, or composition described herein for preventing ischemia-reperfusion related injury in an organ transplant patient is provided, comprising using a binding agent, antibody or antigen binding fragment thereof, or composition disclosed herein prior to, simultaneously with or after providing the organ to the patient. In another embodiment, a use of a binding agent, antibody or antigen binding fragment thereof, or composition described herein for the manufacture of a medicament for preventing ischemia-reperfusion related injury in an organ transplant patient is provided, comprising a binding agent, antibody or antigen binding fragment thereof, or composition disclosed herein for use prior to, simultaneously with or after providing the organ to the patient. In another embodiment, the binding agent, antibody or antigen binding fragment thereof, or composition described herein is for use in preventing ischemia-reperfusion related injury in an organ transplant patient is provided, comprising using a binding agent, antibody or antigen binding fragment thereof, or composition disclosed herein prior to, simultaneously with or after providing the organ to the patient.

In another embodiment, an organ harvesting kit is disclosed comprising a binding agent, antibody, antigen binding fragment thereof, or composition that binds to DPEP-1 disclosed herein. Optionally, the organ harvesting kit may contain or more additional agents.

While not to be bound by any particular mechanism, the protective effects of the binding agents, antibodies, antigen binding fragments, or compositions provided herein are mediated through binding at the DPEP-1 target and a direct reduction in DPEP-1-regulated leukocyte recruitment, inflammation and tumor cell adhesion. These effects described herein on inflammation-mediated disease and tumor metastasis occur independent of DPEP-1 dipeptidase activity or its role in regulating tubular transport. Previous studies have required combination of a DPEP-1 antagonists to prolong the half-life of an antibiotic compound to treat bacterial infection. Other studies have used a DPEP-1 antagonist cilastatin to prevent or treat organ damage by preventing the renal tubular uptake of chemotherapeutic agents, or other nephrotoxic agents (Humanes et al., Kidney Intl, 82:652-553 (2012); Koller et al., Biochem Biophys Res Comm 131(2):974-979 (1985)). As such, in certain embodiments, the binding agents, antibodies, antigen binding fragments or compositions described herein are not used to treat or reduce tissue damage induced directly by toxic compounds such as nephrotoxic compounds or chemotherapeutic agents. In other embodiments, the binding agents, antibodies, antigen binding fragments or compositions described herein are not administered or used in combination with beta-lactam antibiotic compounds. In other embodiments, the binding agents, antibodies, antigen binding fragments or compositions described herein are not administered or used in combination with carbapenem antibiotic compounds. Nonetheless, the binding agents, antibodies, antigen binding fragments or compositions described herein can be used to treat or reduce inflammation in kidney caused by toxic compounds such as nephrotoxic compounds or chemotherapeutic agents. Accordingly, in some embodiments, the binding agents, antibodies, antigen binding fragments or compositions described herein are used to treat or reduce inflammation induced by toxic compounds such as nephrotoxic compounds or chemotherapeutic agents.

The disclosure provides a method to reduce or modulate inflammation comprising administering an effective amount of a binding agent, antibody, antigen binding fragment thereof, or composition that binds to DPEP-1 to reduce or modulate inflammation in a subject. The disclosure also provides a use of a binding agent, antibody or antigen binding fragment thereof or composition described herein to reduce or modulate inflammation in a subject. The disclosure also provides a use of a binding agent, antibody or antigen binding fragment thereof or composition described herein for the manufacture of a medicament for reducing or modulating inflammation. The disclosure further provides a binding agent, antibody or antigen binding fragment thereof or composition described herein for use in reducing or modulating inflammation.

In one embodiment, the composition comprises a binding agent, antibody or antigen binding fragment, and/or a small molecule compound.

In one embodiment, the inflammation is associated with an inflammatory disorder is selected from the group consisting of gastritis, gout, gouty arthritis, arthritis, rheumatoid arthritis, kidney failure, lupus, asthma, psoriasis, pancreatitis, allergy, fibrosis, surgical complications, anemia, fibromyalgia, cancer, heart attack, congestive heart failure, stroke, aortic valve, arteriosclerosis, osteoporosis, multiple sclerosis, Alzheimer's disease, Parkinson's disease, ulcers, chronic bronchitis, asthma, allergy, acute lung injury, pulmonary inflammation, airway hyper-responsiveness, vasculitis, septic shock, inflammatory skin disorders, psoriasis, atopic dermatitis, eczema, and inflammatory bowel disease. In some embodiments, the inflammatory bowel disease is Crohn's disease or ulcerative colitis.

The disclosure provides a method to block leukocyte recruitment of a subject comprising administering an effective amount of a binding agent, antibody or antigen binding fragment thereof, or composition that binds to DPEP-1 to block leukocyte recruitment. The disclosure also provides a use of a binding agent, antibody or antigen binding fragment thereof or composition described herein for blocking leukocyte recruitment of a subject. The disclosure also provides a use of a binding agent, antibody or antigen binding fragment thereof or composition described herein for the manufacture of a medicament for blocking leukocyte recruitment of a subject. The disclosure further provides a binding agent, antibody or antigen binding fragment thereof or composition described herein for use in blocking leukocyte recruitment of a subject.

In one embodiment, the method or use further comprises identifying a subject in need of treatment by diagnostic test for needing reduction in inflammation. In some embodiments, indications for treatment include, but not limited to, clinical signs and symptoms in any patient that is at risk for acute kidney injury (pre-operatively or before administering or use of intravenous contrast) or in any patient having decreasing urine output or increasing serum creatinine, such as in a patient with a systemic infection or low blood pressure.

The disclosure provides a method for reducing or preventing tumor metastasis in a subject comprising administering an effective amount of a binding agent, antibody or antigen binding fragment thereof, or a composition that binds to DPEP-1 thereby reducing or preventing tumor metastasis. The disclosure also provides a use of a binding agent, antibody or antigen binding fragment thereof or composition described herein for reducing or preventing tumor metastasis in a subject. The disclosure also provides a use of a binding agent, antibody or antigen binding fragment thereof or composition described herein for the manufacture of a medicament for reducing or preventing tumor metastasis in a subject. The disclosure further provides a binding agent, antibody or antigen binding fragment thereof or composition described herein for use in reducing or preventing tumor metastasis in a subject. In one embodiment, DPEP-1 acts as an adhesion molecule for leukocytes on tumor cells independent of its enzymatic activity and binding DPEP-1 by a selective DPEP-1 binding agent described herein reduces or prevents tumor metastasis. In another embodiment, DPEP-1 contributes to inflammation which promotes tumor metastasis and binding of DPEP-1 by selective DPEP-1 binding agents reduces or prevents tumor metastasis.

In certain embodiments, the tumor is selected from those tumors known to cause cancer that have the potential to, or are presently capable, of metastasis. For example, the cancer can be pancreatic cancer, kidney cancer, urogenital cancer, melanoma, prostate carcinoma, lung carcinomas, breast carcinomas, thyroid carcinomas, brain cancers, ovarian carcinomas, cervical cancers, uterine endometrial carcinoma, primary peritoneal carcinoma, mesothelioma, eye cancer, muscle, lymphomas, esophageal cancer, gastric cancers, liver cancers, small intestinal tumors, colon cancer, testicular cancer, skin cancers, or adrenal carcinoma. In an embodiment, the kidney cancer is renal cell carcinoma (RCC). In an embodiment, the urogenital cancer is urothelial carcinomas in urinary bladder, kidney, pelvic or ureter. In an embodiment, the lung carcinomas is non-small cell carcinoma, small cell carcinoma, or neuroendocrine carcinoma. In an embodiment, the neuroendocrine carcinoma is carcinoid tumor. In an embodiment, the breast carcinoma is ductal carcinoma, lobular carcinoma, or mixed ductal and lobular carcinoma. In an embodiment, the thyroid carcinomas is papillary thyroid carcinoma, follicular carcinoma, or medullary carcinoma. In an embodiment, the brain cancer is meningioma, astrocytoma, glioblastoma, cerebellum tumors, or medulloblastoma. In an embodiment, the ovarian carcinoma is serous, mucinous, or endometrioid type. In an embodiment, the cervical cancer is squamous cell carcinoma in situ, invasive squamous cell carcinoma, or endocervical adenocarcinoma. In an embodiment, the uterine endometrial carcinoma is endometrioid, serous, or mucinous type. In an embodiment, the mesothelioma is pleural or peritoneal. In an embodiment, the eye cancer is retinoblastoma. In an embodiment, the muscle cancer is rhabdosarcoma or leiomyosarcoma. In an embodiment, the esophageal cancer is adenocarcinoma or squamous cell carcinoma. In an embodiment, the gastric cancer is gastric adenocarcinoma or gastrointestinal stroma tumor. In an embodiment, the liver cancer is hepatocellular carcinoma or bile duct cancer. In an embodiment, the small intestinal tumor is small intestinal stromal tumor or carcinoid tumor. In an embodiment, the colon cancer is adenocarcinoma of the colon, colon high grade dysplasia, or colon carcinoid tumor. In an embodiment, the skin cancer is melanoma or squamous cell carcinoma. In one embodiment, the method or use further comprises identifying a subject in need of treatment through diagnostic tests to determine a need for reduction or prevention of tumor metastasis by determining the presence of a DPEP-1 binding molecule on a tumor of a patient.

The disclosure provides a method for reducing or preventing leukocyte recruitment and inflammation during sepsis in a subject comprising administering an effective amount of a binding agent, antibody, antigen binding fragment, or composition that binds to DPEP-1 thereby reducing or preventing the organ complications of sepsis. The disclosure also provides a use of a binding agent, antibody or antigen binding fragment thereof or composition described herein for reducing or preventing leukocyte recruitment and inflammation during sepsis in a subject. In some embodiments, the use reduces or prevents organ complications of sepsis. The disclosure also provides a use of a binding agent, antibody or antigen binding fragment thereof or composition described herein for the manufacture of a medicament for reducing or preventing leukocyte recruitment and inflammation during sepsis in a subject. In some embodiments, the medicament reduces or prevents organ complications of sepsis. The disclosure further provides a binding agent, antibody or antigen binding fragment thereof or composition described herein for use in reducing or preventing leukocyte recruitment and inflammation during sepsis in a subject. In some embodiments, the binding agent, antibody or antigen binding fragment thereof or composition that binds to DPEP-1 is for use in reducing or preventing organ complications of sepsis.

In one embodiment, the method or use further comprises identifying a subject in need of treatment through diagnostic test to determine a need for reduction or prevention of ischemia-reperfusion injury. Indications for treatment include, but not limited to, clinical signs and symptoms of ischemia-reperfusion injury or undergoing a surgical procedure with a high risk of ischemia-reperfusion injury.

The disclosure includes a method of treating a symptom of ischemia-reperfusion injury in a subject comprising administering to the patient a pharmaceutically effective amount of a binding agent, antibody or antigen binding fragment thereof, or composition that binds to DPEP-1. The disclosure also includes a use of a binding agent, antibody or antigen binding fragment thereof or composition described herein for treating a symptom of ischemia-reperfusion injury in a subject. The disclosure also includes a use of a binding agent, antibody or antigen binding fragment thereof or composition described herein for the manufacture of a medicament for treating a symptom of ischemia-reperfusion injury in a subject. The disclosure further includes a binding agent, antibody or antigen binding fragment thereof or composition described herein for use in treating a symptom of ischemia-reperfusion injury in a subject.

In one embodiment, the binding agent, antibody or antigen binding fragment, or composition is administered or for use until symptoms of ischemia-reperfusion injury are reduced or ameliorated.

In one embodiment, the isolated binding agent, antibody or antigen binding fragment thereof or variant thereof is administered or for use at a dosage between about 0.1 μg/kg to 100 mg/kg. In one embodiment, the dosage is between 2 μg and 10 g.

The disclosure provides a method for reducing or preventing ischemia-reperfusion injury related disorders in a subject comprising administering an effective amount of a binding agent, antibody or antigen binding fragment thereof, or composition that binds to DPEP-1 thereby reducing or preventing ischemia-reperfusion injury. The disclosure also provides a use of a binding agent, antibody or antigen binding fragment thereof or composition described herein for reducing or preventing ischemia-reperfusion injury related disorders in a subject. The disclosure also provides a use of a binding agent, antibody or antigen binding fragment thereof or composition described herein for the manufacture of a medicament for reducing or preventing ischemia-reperfusion injury related disorders in a subject. The disclosure further provides a binding agent, antibody or antigen binding fragment thereof or composition described herein for use in reducing or preventing ischemia-reperfusion injury related disorders in a subject.

In one embodiment, the method or use reduces or prevents the leukocyte recruitment and inflammation that is associated with ischemia-reperfusion injury.

In one embodiment, the ischemia-reperfusion injury related disorder is associated with ischemic and post-ischemic events in organs and tissues, and the disorder is selected from a group consisting of thrombotic stroke, myocardial infarction, angina pectoris, embolic vascular occlusions, peripheral vascular insufficiency, splanchnic artery occlusion, arterial occlusion by thrombi, arterial occlusion by embolisms, arterial occlusion by non-occlusive processes, mesenteric arterial occlusion, mesenteric vein occlusion; ischemia-reperfusion injury to the mesenteric microcirculation; ischemic acute renal failure, ischemia-reperfusion injury to the cerebral tissue, intestinal intussusception, hemodynamic shock, tissue dysfunction, organ failure, restenosis, atherosclerosis, thrombosis, platelet aggregation, shock liver, spinal cord injury, or brain injury. In an embodiment, the arterial occlusion by non-occlusive processes is arterial occlusion following low mesenteric flow or sepsis. In an embodiment, the organ failure is heart failure, liver failure, kidney failure, or the like. In an embodiment, the ischemia-reperfusion injury is resulted from a surgical procedure. In an embodiment, the surgical procedure is peri-operative procedure, cardiac surgery, organ surgery, organ transplantation, angiography, cardiopulmonary, or cerebral resuscitation. In an embodiment, the ischemia-reperfusion injury is associated with harvesting donor organs for transplantation. In an embodiment, the ischemia-reperfusion injury occurs to allograft organs during donor procurement, ex vivo handling, or implantation into a transplant recipient.

In one embodiment, the ischemia-reperfusion injury is associated with harvesting donor organs for transplantation.

In one embodiment, the ischemia-reperfusion injury occurs to allograft organs during donor procurement, ex vivo handling or implantation into a transplant recipient.

In various embodiments, the binding agents, antibodies, antigen binding fragments, or compositions can be administered or for use (i) prior to, during or following harvesting a donor organ which will be transplanted or (ii) prior to or during a surgical procedure in which ischemia is expected.

The disclosure provides a method for reducing or preventing acute kidney injury in a subject comprising administering an effective amount of a binding agent, antibody or antigen binding fragment thereof, or composition that binds to DPEP-1 thereby reducing or preventing acute kidney injury. The disclosure also provides a use of a binding agent, antibody or antigen binding fragment thereof or composition described herein for reducing or preventing acute kidney injury in a subject. The disclosure also provides a use of a binding agent, antibody or antigen binding fragment thereof or composition described herein for the manufacture of a medicament for reducing or preventing acute kidney injury in a subject. The disclosure further provides a binding agent, antibody or antigen binding fragment thereof or composition described herein for use in reducing or preventing acute kidney injury in a subject. In one embodiment, the method or use reduces or prevents the leukocyte recruitment and inflammation that is associated with acute kidney injury.

In one embodiment, the method or use comprises identifying a subject in need of treatment through diagnostic test to determine a need for reduction or prevention of acute kidney injury. Acute kidney injury can be caused by ischemia reperfusion, sepsis, pigment, toxin, or drug. In one embodiment, the method or use comprises treating acute kidney injury. In one embodiment, the acute kidney injury comprises ischemia reperfusion-induced condition, pigment-induced condition, toxin-induced condition, or drug-induced condition. In one embodiment, the acute kidney injury is a result of ischemia reperfusion. In one embodiment, the acute kidney injury is a result of sepsis. In one embodiment, the acute kidney injury is a result of pigment. In one embodiment, the acute kidney injury is a result of toxin. In one embodiment, the acute kidney injury is a result of drug.

The pigment that causes acute kidney injury can be myoglobin or heme, which is a component of hemoglobin. In one embodiment, the acute kidney injury is toxin-induced kidney injury. In one embodiment, the acute kidney injury is drug-induced kidney injury. In one embodiment, the acute kidney injury is pigment-induced kidney injury. In one embodiment, the pigment is myoglobin or hemoglobin. In one embodiment, the pigment is heme.

In one embodiment, the acute kidney injury is contrast-induced kidney injury.

Inflammation also plays a role in chronic kidney disease and DPEP-1 could be a target to reduce inflammation in chronic kidney disease. According, in one embodiment, the method or use comprises identifying a subject in need of treatment through diagnostic test to determine a need for reduction or prevention of chronic kidney disease. In one embodiment, the method or use comprises treating chronic kidney disease. In one embodiment, the chronic kidney disease is associated with diabetes mellitus, hypertension, or glomerulonephritis. In one embodiment, the chronic kidney disease is a vascular disease, a glomerular disease, a tubulointerstitial disease, or an obstructive nephropathy. In one embodiment, the chronic kidney disease is a vascular disease. In one embodiment, the vascular disease is large vessel disease or small vessel disease. In one embodiment, the large vessel disease is bilateral kidney artery stenosis. In one embodiment, the small vessel disease is ischemic nephropathy, hemolytic-uremic syndrome, or vasculitis. In one embodiment, the chronic kidney disease is a glomerular disease. In one embodiment, the glomerular disease is primary glomerular disease or secondary glomerular disease. In one embodiment, the primary glomerular disease is focal segmental glomerulosclerosis or IgA nephropathy. In one embodiment, the secondary glomerular disease is diabetic nephropathy or lupus nephritis. In one embodiment, the chronic kidney disease is a tubulointerstitial disease. In one embodiment, the tubulointerstitial disease is drug-induced chronic tubulointerstitial nephritis, toxin-induced chronic tubulointerstitial nephritis, or reflux nephropathy. In one embodiment, the chronic kidney disease is an obstructive nephropathy. In one embodiment, the obstructive nephropathy is bilateral kidney stones or benign prostatic hyperplasia of the prostate gland. In one embodiment, the chronic kidney disease is polycystic kidney disease, 17q12 microdeletion syndrome, or Mesoamerican nephropathy.

In certain embodiments, the binding agent, antibody, antigen binding fragment, or composition may be administered or for use in combination with one or more additional therapeutic agents. Co-administration or use includes simultaneous administration or use in separate compositions (also referred to as concurrent administration), administration or use at different times in separate compositions, or administration or use in a composition in which both agents are present.

In one embodiment, the at least one additional therapeutic agent is selected from the group consisting of chemotherapeutic or anti-proliferative agents, antiviral, antibiotic, antihistamine, an emollient, systemic phototherapy, psoralen photochemotherapy, laser therapy, hormone replacement therapy, an anti-inflammatory agent, an immunomodulatory or immunosuppressive agent, a neurotrophic factor, an agent for treating cardiovascular disease, an agent for treating diabetes, an agent for treating immunodeficiency disorders, and immune checkpoint inhibitors.

### VI. Routes of Administration

A composition comprising a binding agent, antibody or antigen binding fragment thereof that binds to DPEP-1 as described herein may be administered or used by any appropriate route. In some embodiments, the binding agent, antibody or antigen binding fragment thereof, or the composition is administered or used parenterally. In some embodiments, the parenteral administration is selected from intravenous, intradermal, inhalation, transdermal (topical), intraocular, intramuscular, subcutaneous, intramuscular, and/or transmucosal administration. In some embodiments, the composition as described herein is administered or used subcutaneously. As used herein, the term “subcutaneous tissue”, is defined as a layer of loose, irregular connective tissue immediately beneath the skin. For example, the subcutaneous administration may be performed by injecting a composition into areas including, but not limited to, thigh region, abdominal region, gluteal region, or scapular region. In some embodiments, the binding agent, antibody or antigen binding fragment thereof, or the composition as described herein is administered or used intravenously. In other embodiments, a binding agent, antibody or antigen binding fragment thereof, or a composition that binds to DPEP-1 as described herein is administered or used by direct administration to a target tissue, such as heart or muscle (e.g., intramuscular), tumor (intratumorally), nervous system (e.g., direct injection into the brain; intraventricularly; intrathecally). Alternatively, a binding agent, antibody or antigen binding fragment thereof, or a composition that binds to DPEP-1 as described herein (or a composition or medicament containing a DPEP-1 binding antibody or antigen binding fragment thereof as described herein) can be administered or used by inhalation, parenterally, intradermally, transdermally, or transmucosally (e.g., orally or nasally). More than one route can be used concurrently, if desired.

In some embodiments, a binding agent, antibody or antigen binding fragment thereof, or a composition that binds to DPEP-1 as described herein is administered or used orally. In some embodiments, the present disclosure provides solid dosage forms of binding agent, antibody or antigen binding fragment thereof that binds to DPEP-1 as described herein for oral administration including (a) a binding agent, antibody or antigen binding fragment thereof that binds to DPEP-1, (b) at least one pharmaceutically acceptable pH-lowering agent, (c) at least one absorption enhancer effective to promote bioavailability of the binding agent, antibody or antigen binding fragment thereof that binds to DPEP-1, and (d) a protective vehicle. In some embodiments, the solid dosage form is a capsule or tablet.

### VII. Dosing

An effective quantity of a binding agent, antibody or antigen binding fragment thereof, or a composition that binds to DPEP-1 of interest is employed in treatment. The dosage of antibodies or fragments thereof used in accordance with the disclosure varies depending on the antibody or fragment thereof and the condition being treated. The dose is sufficient to ameliorate symptoms or signs of the disease treated without producing unacceptable toxicity to the patient. In general, an effective amount of the antibody or fragment thereof is that which provides either subjective relief of symptoms or an objectively identifiable improvement.

Various embodiments include differing dosing regimen. In some embodiments, the binding agent, antibody or antigen binding fragment thereof, or the composition that binds to DPEP-1 is administered or used via continuous infusion. In some embodiments, the continuous infusion is intravenous. In other embodiments, the continuous infusion is subcutaneous. Alternatively or additionally, in some embodiments, the binding agent, antibody or antigen binding fragment thereof, or the composition that binds to DPEP-1 is administered bimonthly, monthly, twice monthly, triweekly, biweekly, weekly, twice weekly, thrice weekly, daily, twice daily, or on another clinically desirable dosing schedule. The dosing regimen for a single subject need not be at a fixed interval, but can be varied over time, depending on the needs of the subject.

In one embodiment, the local dosage is administered or used at least once a day until a therapeutic result is achieved. The dosage can be administered or used twice a day, but more or less frequent dosing can be administered. Once a therapeutic result is achieved, the binding agent, antibody or antigen binding fragment thereof, or the composition can be tapered or discontinued. Occasionally, side effects warrant discontinuation of therapy. An effective quantity of the binding agent, antibody or antigen binding fragment thereof, or the composition of interest is employed in treatment.

When employed as pharmaceuticals, the antibodies or fragments thereof of the present disclosure are administered or used in the form of pharmaceutical compositions and these pharmaceutical compositions represent further embodiments of the present disclosure. These antibodies or fragments thereof can be administered or used by a variety of routes including oral, rectal, transdermal, subcutaneous, intravenous, intramuscular, and intranasal, or via intratracheal instillation or aerosol inhalation.

The antibodies, fragments thereof, or compositions that bind to DPEP-1 are useful in reducing inflammation or modifying the inflammatory profile of a tissue, e.g., into the liver. The manner of administration will be defined by the application of the compound and can be determined by methods of clinical testing to find the optimum dose.

In one embodiment, the dosage is between about 0.01 mg/kg to about 100 mg/kg of active binding agent, antibody or antigen binding fragment thereof, between about 0.01 mg/kg to about 50 mg/kg, between about 0.01 mg/kg to about 25 mg/kg, or about 0.5 mg/kg to about 10 mg/kg.

In other embodiments, the dosage is between about 0.1 mg/kg to about 100 mg/kg, between about 0.1 mg/kg to about 50 mg/kg, between about 0.1 mg/kg to about 25 mg/kg, or between about 0.1 mg/kg to about 10 mg/kg.

In other embodiments, the dosage is between about 0.5 mg/kg to about 100 mg/kg, about 0.5 mg/kg to about 50 mg/kg, about 0.5 mg/kg to about 25 mg/kg, or about 0.5 mg/kg to about 10 mg/kg.

In other embodiments, the dosage is between about 1.0 mg/kg to about 25 mg/kg, between about 1.0 mg/kg to about 50 mg/kg, between about 1.0 mg/kg to about 70 mg/kg, between about 1.0 mg/kg to about 100 mg/kg, between about 5.0 mg/kg to about 25 mg/kg, between about 5.0 mg/kg to about 50 mg/kg, between about 5.0 mg/kg to about 70 mg/kg, between about 5.0 mg/kg to about 100 mg/kg, between about 10.0 mg/kg to about 25 mg/kg, between about 10.0 mg/kg to about 50 mg/kg, between about 10.0 mg/kg to about 70 mg/kg, or between about 10.0 mg/kg to about 100 mg/kg.

In one embodiment, the dosage is between about 0.2 mg to about 10 g of active binding agent, antibody or antigen binding fragment thereof, between about 0.2 mg to about 5 g, between about 0.2 mg to about 2.5 g, between about 0.2 mg to about 2 g, between about 0.2 mg to about 1 g, between about 0.2 mg to about 500 mg, or between about 0.2 mg to about 250 mg.

In one embodiment, the dosage is between about 2 mg to about 10 g of active binding agent, antibody or antigen binding fragment thereof, between about 2 mg to about 5 g, between about 2 mg to about 2.5 g, between about 2 mg to about 2 g, between about 2 mg to about 1 g, between about 2 mg to about 500 mg, or between about 2 mg to about 250 mg.

In one embodiment, the dosage is between about 10 mg to about 10 g of active binding agent, antibody or antigen binding fragment thereof, between about 10 mg to about 5 g, between about 10 mg to about 2.5 g, between about 10 mg to about 2 g, between about 10 mg to about 1 g, between about 10 mg to about 500 mg, or between about 10 mg to about 250 mg.

In other embodiments, the dosage is between about 20 mg to about 500 mg, between about 20 mg to about 1 g, between about 20 mg to about 1.4 g, between about 20 mg to about 1.5 g, between about 20 mg to about 2 g, between about 20 mg to about 2.5 g, between about 20 mg to about 5 g, between about 20 mg to about 10 g, between about 20 mg to about 500 mg, between about 100 mg to about 1 g, between about 100 mg to about 1.5 g, between about 100 mg to about 2 g, between about 100 mg to about 2.5 g, between about 100 mg to about 5 g, between about 100 mg to about 10 g, between about 200 mg to about 1 g, between about 200 mg to about 1.5 g, between about 200 mg to about 2 g, between about 200 mg to about 2.5 g, between about 200 mg to about 5 g, or between about 200 mg to about 10 g.

In other embodiments, the dosage is about 2 μg, 5 μg, 10 μg, 20 μg, 25 μg, 50 μg, 75 μg, 0.1 mg, 0.2 mg, about 0.5 mg, about 1 mg, about 2 mg, about 2.5 mg, about 5 mg, about 10 mg, about 20 mg, about 25 mg, about 50 mg, about 75 mg, about 100 mg, about 150 mg, about 200 mg, about 250 mg, about 300 mg, about 400 mg, about 500 mg, about 750 mg, about 800 mg, about 1 g, about 1.5 g, about 2 g, about 2.5 g, about 3 g, about 4 g, about 5 g, about 6 g, about 7 g, about 8 g, about 9 g, or about 10 g.

In another embodiment, the dosage is between about 50 μM and about 500 μM.

In various embodiments, compositions described herein, or salts thereof, are administered or used in amounts between about 0.001 and about 20 mg/kg body weight per day, between about 0.01 and about 10 mg/kg body weight per day, between about 0.1 and about 1000 μg/kg body weight per day, or between about 0.1 to about 100 μg/kg body weight per day. Routes of administration or usage vary. For example, antibodies described herein, or salts thereof, are administered or used in amounts between about 0.1 and about 1000 μg/kg body weight per day, or between about 0.1 to about 100 μg/kg body weight per day, by subcutaneous injection. By way of example, for a 50 kg human female subject, the daily dose of active ingredient is from about 5 to about 5000 μg, or from about 5 to about 5000 μg by subcutaneous injection. Different doses will be needed, depending on the route of administration or use, the antibody potency, the pharmacokinetic profile and the applicable bioavailability observed, and the active agent and the disease being treated. In an alternate embodiment where the administration or use is by inhalation, the daily dose is from 1000 to about 20,000 μg, twice daily. In other embodiments, the dosage is between about 2 μg/day to about 10 g/day, about 2 μg/day, 5 μg/day, 10 μg/day, 20 μg/day, 25 μg/day, 50 μg/day, 75 μg/day, 0.1 mg/day, 0.2 mg/day, about 0.5 mg/day, about 1 mg/day, about 2 mg/day, about 2.5 mg/day, about 5 mg/day, about 10 mg/day, about 20 mg/day, about 25 mg/day, about 50 mg/day, about 75 mg/day, about 100 mg/day, about 150 mg/day, about 200 mg/day, about 250 mg/day, about 300 mg/day, about 400 mg/day, about 500 mg/day, about 750 mg/day, about 800 mg/day, about 1 g/day, about 1.5 g/day, about 2 g/day, about 2.5 g/day, about 3 g/day, about 4 g/day, about 5 g/day, about 6 g/day, about 7 g/day, about 8 g/day, about 9 g/day, or about 10 g/day.

In other mammals, such as horses, dogs, and cattle, higher doses may be required. This dosage may be delivered in a conventional pharmaceutical composition by a single administration, by multiple applications, or via controlled release, as needed to achieve the most effective results.

### VIII. Kits

In some embodiments, the present disclosure further provides kits or other articles of manufacture which contain a binding agent, antibody or antigen binding fragment thereof, or pharmaceutical composition that binds DPEP-1 described herein, as well as instructions for its reconstitution (if lyophilized) and/or use. Kits or other articles of manufacture may include a container, a syringe, vial and any other articles, devices or equipment useful in administration (e.g., subcutaneous, by inhalation). Suitable containers include, for example, bottles, vials, syringes (e.g., pre-filled syringes), ampules, cartridges, reservoirs, or lyo-jects. The container may be formed from a variety of materials such as glass or plastic. In some embodiments, the container is a pre-filled syringe. Suitable pre-filled syringes include, but not limited to, borosilicate glass syringes with baked silicone coating, borosilicate glass syringes with sprayed silicone, or plastic resin syringes without silicone.

Typically, the container may hold formulations and a label on, or associated with, the container that may indicate directions for reconstitution and/or use. For example, the label may indicate that the formulation is reconstituted to concentrations as described above. The label may further indicate that the formulation is useful or intended for, for example, subcutaneous administration. In some embodiments, the container may contain a single dose of a stable formulation containing an antibody, antigen binding fragment thereof, or a composition that binds DPEP-1. In various embodiments, a single dose of the stable formulation is present in a volume of less than about 15 ml, about 10 ml, about 5.0 ml, about 4.0 ml, about 3.5 ml, about 3.0 ml, about 2.5 ml, about 2.0 ml, about 1.5 ml, about 1.0 ml, or about 0.5 ml. Alternatively, the container holding the formulation may be a multi-use vial, which allows for repeat administrations (e.g., from 2-6 administrations) of the formulation. Kits or other articles of manufacture may further include a second container comprising a suitable diluent (e.g., BWFI, saline, buffered saline). Upon mixing of the diluent and the formulation, the final protein concentration in the reconstituted formulation may be at least about 0.2 μg/ml (e.g., at least about 0.5 μg/ml, at least about 1 μg/ml, at least about 2 μg/ml, at least about 5 μg/ml, at least about 10 μg/ml, at least about 20 μg/ml, at least about 25 μg/ml, at least about 50 μg/ml, at least about 75 μg/ml, at least about 0.1 mg/ml, at least about 0.2 mg/ml, at least about 0.5 mg/ml, at least about 1 mg/ml, at least about 2 mg/ml, at least about 2.5 mg/ml, at least about 5 mg/ml, at least about 10 mg/ml, at least about 20 mg/ml, at least about 30 mg/ml, at least about 40 mg/ml, at least about 50 mg/ml, at least about 75 mg/ml, at least about 100 mg/ml). Kits or other articles of manufacture may further include other materials desirable from a commercial and user standpoint, including other buffers, diluents, filters, needles, syringes, and package inserts with instructions for use. In some embodiments, kits or other articles of manufacture may include an instruction for self-administration.

### IX. Screening Methods

Disclosed herein is a method for screening for compositions, including antibodies, that bind to DPEP-1, for example, human DPEP-1.

In one embodiment, the method comprises: (a) screening a library of test antibodies for their ability to bind to DPEP-1 in the tissue; and (b) identifying antibodies that show selective binding affinity. In certain embodiments, the identified antibodies are subject to one or more additional testing methods.

In one embodiment, the method comprises: (a) screening a library of test antibodies for their ability to bind to DPEP-1 in the tissue; and (b) identifying antibodies that show selective binding affinity. In certain embodiments, the identified antibodies are subject to one or more additional testing methods.

In one embodiment, the screening method comprises identifying an antibody effective to decrease inflammation in a tissue of a patient comprising: (a) screening a library of test antibodies for their ability to bind to DPEP-1 in the tissue; (b) selecting candidate test antibodies that show selective binding affinity; (c) testing the candidate antibodies for inflammation reducing activity, and (d) selecting a candidate antibody if it decrease inflammation, thereby providing an antibody effective to decrease inflammation.

For those library test antibodies that show a selective binding affinity over other test antibodies in the library, e.g., at least a 10⁻¹⁰⁰ fold increase in binding affinity over other antibodies, the antibodies with selective binding affinity is further tested for its ability to reduce inflammation in a tissue, according to methods detailed below. Test antibodies that are shown to reduce inflammation in a tissue are then identified as lead antibodies for further antibody testing and development.

In one embodiment, the tissue is lung tissue, liver tissue or kidney tissue.

In one embodiment, a method is provided for identifying an antibody effective to block leukocyte recruitment in the vasculature of a patient.

In one embodiment, the disclosure provides a method of identifying an antibody effective to reduce inflammation in a tissue of a patient comprising: (a) screening a library of test antibodies for their ability to bind to DPEP-1; (b) selecting antibodies that show selective binding affinity; (c) testing the antibodies for leukocyte recruitment inhibiting activity, and (d) selecting an antibody if it reduces inflammation in a tissue.

In one embodiment, the tissue is lung tissue, liver tissue or kidney tissue.

In one embodiment, the method further comprises the steps of (e) further testing the antibody for its ability to block leukocyte recruitment in an animal bearing a solid tumor; and (f) selecting the antibody if it block leukocyte recruitment in step (e).

In one embodiment, the method further comprises the steps of (e) further testing the antibody for its ability to inhibit tumor metastasis in an animal bearing a solid tumor; and (f) selecting the antibody if it inhibits tumor metastasis in step (e).

In one embodiment, the method further comprises the steps of (e) further testing the antibody for its ability to inhibit tumor metastasis to the lungs and liver in an animal bearing a solid tumor known to metastasize the lungs or liver; and (f) selecting the antibody if it inhibits tumor metastasis in step (e).

In one embodiment, the method further comprises the steps of (e) further testing the antibody for its ability to treat sepsis in a subject; and (f) selecting the antibody if it treats sepsis in step (e).

In one embodiment, the method further comprises the steps of (e) further testing the antibody for its ability to treat bacterial sepsis in a subject; and (f) selecting the antibody if it treats sepsis in step (e).

In one embodiment, the method further comprises the steps of (e) further testing the antibody for its ability to treat acute kidney damage in a subject; and (f) selecting the antibody if it treats acute kidney damage in step (e).

In one embodiment, step (a) in the method includes screening a library of test antibodies for their ability to bind to DPEP-1.

In one embodiment, the method further comprises identify other secondary targets involved in inflammation including co-factors, co-receptors, circulating factors and accessory proteins by using the binding agent, antibody or antigen binding fragment thereof that binds to DPEP-1 in a protein microarray.

The following non-limiting Examples are illustrative of the present disclosure:

### EXAMPLES

### Example 1. Antibodies that Bind to Human DPEP-1

A male llama (Lama glama) was immunized with recombinant human DPEP-1 (hDPEP-1; accession number: P16444; NCBI Reference Sequence: NM_004413) ectodomain (amino acids 17-385). Briefly, the animal was immunized subcutaneously three times with 200 μg of hDPEP-1 ectodomain (days 0, 21, and 28). The priming immunization was adjuvanted with complete Freund's adjuvant and boost immunizations were adjuvanted with incomplete Freund's adjuvant. Blood samples were collected on days 0, 28, and 35, from which serum was obtained after clotting and peripheral blood mononuclear cells (PBMCs) were purified by density gradient centrifugation. The resulting serum from the immunized bleeds was specific to hDPEP-1 and was shown to have a strong, positive immune response against human DPEP-1 from two different sources (CreativeBioMart and SinoBiologicals). PBMCs were isolated from the llama and an immune V_{H}H phage display library in the phagemid vector pMED1 was built (see Hussack et al., “Neutralization of Clostridium difficile toxin A with single-domain antibodies targeting the cell receptor binding domain.” J Biol Chem. 286:8961-8976 (2011); herein incorporated by reference). Ninety-four clones were amplified for the generation of monoclonal phages and the specificity against hDPEP1 was evaluated by ELISA. The clones were sequenced with V_{H}Hs-specific primers for the identification of unique sequences and nine different sequences were identified. Transference of these sequences were made from the phagemid vector to pMRo. BAP.H6, a vector that allows the expression of the monomeric V_{H}Hs fused to a Biotinylation Acceptor Peptide (BAP) and His6 tags (see Rossotti 2015 et al, “Streamlined method for parallel identification of single domain antibodies to membrane receptors on whole cells”, Biochim Biophys Acta, 1850(7): 1397-404; herein incorporated by reference). The His6 is for purification by NiNTA chromatography and the BAP permits site specific biotinylation for detection by streptavidin. The nine different sequences identified are shown in Table 1 (sdABP01-09 with BAP and His6 tags; SEQ ID NOs: 1-9) and Table 2A (sdABP01-09 without BAP and His6 tags; SEQ ID NOs: 12-20). The CDRs of these sdABs are shown in Table 2β.

TABLE 1
sdABP01 with BAP and His6 tags.

| SEQ ID | |
|---|---|
| NO | Amino Acid Sequence |
| SEQ ID | EVQLVESGGGLVQPGGSLRLSCAASGSTLNWYTIGWFRQAPGKEREEVS |
| NO: 1 | CISSSGGSTKYADSVKGRFTISRSNALNTVYLQMNTLKPDDTAVYYCAL |
| (sdABP01) | DLDSAFCGSHISEYEYWGQGTQVTVSSGQAGQGGGLNDIFEAQKIEWHE |
| | LEHHHHHH |
| SEQ ID | EVQLVESGGGSVQAGGSLRLSCVASGIHFGSHSMAWYRQAPGKERDLVA |
| NO: 2 | RISALGNTNYANSVKGRFTISRDTNKSTLYLQMNTLKPEDTAMYYCAPW |
| (sdABP02) | SAYDREGDFRSWGQGTQVTVSSGQAGQGGGLNDIFEAQKIEWHELEHHH |
| | HHH |
| SEQ ID | EVQLVESGGGLVQPGGSLRLSCATSEFTLDYYAIGWFRQAPGKEREGVS |
| NO: 3 | CISSSGGTTNYADSVKGRFTISSDNAKNTVSLQMNSLRPEDTAVYYCAA |
| (sdABP03) | ARVSAYYLGNYGCLNAEYGYWGQGTQVTVSSGQAGQGGGLNDIFEAQKI |
| | EWHELEHHHHHH |
| SEQ ID | EVQLVESGGGLVQPGGSLRLSCAASGFTFSSYYMSWVRQAPGKGPEWVS |
| NO: 4 | GINTDGDDTSYADSVKGRFTISRDNAKNTLYLQMSSLKPEDTALYYCAR |
| (sdABP04) | AARSGSTTWGRNYWGQGTQVTVSSGQAGQGGGLNDIFEAQKIEWHELEH |
| | HHHHH |
| SEQ ID | EVQLVESGGGLVQPGGSLRLSCAASGFTFSTYAMTWVRKAPGKGFEWIS |
| NO: 5 | SIDSGGGVTLYADSVKGRFTISKDNAKNTLYLQMNNLKPDDTAVYYCVK |
| (sdABP05) | NYGSTSLQSRGQGTQVTVSSGQAGQGGGLNDIFEAQKIEWHELEHHHHH |
| | H |
| SEQ ID | EVQLVDSGGGLVQPGGSLRLSCAASGFTFSNYDMSWVRQGPGKGPEWVS |
| NO: 6 | IISYVGGLTRYSDSVKGRFTISRDNAKNTLYLQMNSLNTEDTALYYCAR |
| (sdABP06) | VKSMHPTSTTGEYDYRGRGTQVTVSSGQAGQGGGLNDIFEAQKIEWHEL |
| | EHHHHHH |
| SEQ ID | EVQLVESGGGLVEYGGSLRLSCAASESTLDNYAIAWFRQAPGKEREVVS |
| NO: 7 | CVGKSGGRSDYADSVKGRFTISRDNAKNTVYLQMNSLKPEDTAVYSCAA |
| (sdABP07) | RRVWFGGCVLGTSQGQYDYWGQGTQVTVSSGQAGQGGGLNDIFEAQKIE |
| | WHELEHHHHHH |
| SEQ ID | EVQLVSSGGGLVQPGGSLRLSCKASRFTLERYTIGWFRQAPGKEREGIA |
| NO: 8 | CISSSGGDTNYADSVKGRFTISRDNVVEKVYLQMDSLKPEDTAVYYCAA |
| (sdABP08) | RTYACDYKSRWLTYEFRGQGTQVTVSSGQAGQGGGLNDIFEAQKIEWHE |
| | LEHHHHHH |
| SEQ ID | QVQLVESGGGLVQPGGSLRLSCAASGFTFSSYGMTWVRQAPGKGFEWVS |
| NO: 9 | TISISGSRTTYAGSVKDRFTISRDNAKNTLYLQMNSLKPEDTAVYYCRN |
| (sdABP09) | ILVQGQGTQVTVSSGQAGQGGGLNDIFEAQKIEWHELEHHHHHH |

The underlined sequences are the V_{H}Hs, which are separately shown in Table 2A. The V_{H}H is linked to the BAP sequence through a short spacer GQAGQGG (which is encoded by a sequence comprising a SFII restriction site that is used during the cloning steps). The BAP sequence is GLNDIFEAQKIEWHE (SEQ ID NO: 10) which is linked to the His6 tag (HHHHHH; SEQ ID NO: 11) with a spacer (LE). The amino acid sequences in bold are CDRs, which are separately shown in Table 2β.

TABLE 2A
sdABP01 without short spacer, BAP, and His6 tags.

| SEQ ID | |
|---|---|
| NO | Amino Acid Sequence |
| SEQ ID | EVQLVESGGGLVQPGGSLRLSCAASGSTLNWYTIGWFRQAPGKEREEVS |
| NO: 12 | CISSSGGSTKYADSVKGRFTISRSNALNTVYLQMNTLKPDDTAVYYCAL |
| (sdABP01) | DLDSAFCGSHISEYEYWGQGTQVTVSS |
| SEQ ID | EVQLVESGGGSVQAGGSLRLSCVASGIHFGSHSMAWYRQAPGKERDLVA |
| NO: 13 | RISALGNTNYANSVKGRFTISRDTNKSTLYLQMNTLKPEDTAMYYCAPW |
| (sdABP02) | SAYDREGDFRSWGQGTQVTVSS |
| SEQ ID | EVQLVESGGGLVQPGGSLRLSCATSEFTLDYYAIGWFRQAPGKEREGVS |
| NO: 14 | CISSSGGTTNYADSVKGRFTISSDNAKNTVSLQMNSLRPEDTAVYYCAA |
| (sdABP03) | ARVSAYYLGNYGCLNAEYGYWGQGTQVTVSS |
| SEQ ID | EVQLVESGGGLVQPGGSLRLSCAASGFTFSSYYMSWVRQAPGKGPEWVS |
| NO: 15 | GINTDGDDTSYADSVKGRFTISRDNAKNTLYLQMSSLKPEDTALYYCAR |
| (sdABP04) | AARSGSTTWGRNYWGQGTQVTVSS |
| SEQ ID | EVQLVESGGGLVQPGGSLRLSCAASGFTFSTYAMTWVRKAPGKGFEWIS |
| NO: 16 | SIDSGGGVTLYADSVKGRFTISKDNAKNTLYLQMNNLKPDDTAVYYCVK |
| (sdABP05) | NYGSTSLQSRGQGTQVTVSS |
| SEQ ID | EVQLVDSGGGLVQPGGSLRLSCAASGFTFSNYDMSWVRQGPGKGPEWVS |
| NO: 17 | IISYVGGLTRYSDSVKGRFTISRDNAKNTLYLQMNSLNTEDTALYYCAR |
| (sdABP06) | VKSMHPTSTTGEYDYRGRGTQVTVSS |
| SEQ ID | EVQLVESGGGLVEYGGSLRLSCAASESTLDNYAIAWFRQAPGKEREVVS |
| NO: 18 | CVGKSGGRSDYADSVKGRFTISRDNAKNTVYLQMNSLKPEDTAVYSCAA |
| (sdABP07) | RRVWFGGCVLGTSQGQYDYWGQGTQVTVSS |
| SEQ ID | EVQLVSSGGGLVQPGGSLRLSCKASRFTLERYTIGWFRQAPGKEREGIA |
| NO: 19 | CISSSGGDTNYADSVKGRFTISRDNVVEKVYLQMDSLKPEDTAVYYCAA |
| (sdABP08) | RTYACDYKSRWLTYFFRGQGTQVTVSS |
| SEQ ID | QVQLVESGGGLVQPGGSLRLSCAASGFTFSSYGMTWVRQAPGKGFEWVS |
| NO: 20 | TISISGSRTTYAGSVKDRFTISRDNAKNTLYLQMNSLKPEDTAVYYCRN |
| (sdABP09) | ILVQGQGTQVTVSS |

The amino acid sequences in bold are CDRs, which are separately shown in Table 2β.

TABLE 2B
CDR Sequences.

| SEQ ID NO | CDR Sequence |
|---|---|
| SEQ ID NO: 21 | GSTLNWYT |
| (sdABP01-CDR1) | |
| SEQ ID NO: 22 | ISSSGGST |
| (sdABP01-CDR2) | |
| SEQ ID NO: 23 | ALDLDSAFCGSHISEYEY |
| (sdABP01-CDR3) | |
| SEQ ID NO: 24 | GIHFGSHS |
| (sdABP02-CDR1) | |
| SEQ ID NO: 25 | ISALGNT |
| (sdABP02-CDR2) | |
| SEQ ID NO: 26 | APWSAYDREGDERS |
| (sdABP02-CDR3) | |
| SEQ ID NO: 27 | EFTLDYYA |
| (sdABP03-CDR1) | |
| SEQ ID NO: 28 | ISSSGGTT |
| (sdABP03-CDR2) | |
| SEQ ID NO: 29 | AAARVSAYYLGNYGCLNAEYGY |
| (sdABP03-CDR3) | |
| SEQ ID NO: 30 | GFTFSSYY |
| (sdABP04-CDR1) | |
| SEQ ID NO: 31 | INTDGDDT |
| (sdABP04-CDR2) | |
| SEQ ID NO: 32 | ARAARSGSTTWGRNY |
| (sdABP04-CDR3) | |
| SEQ ID NO: 33 | GFTFSTYA |
| (sdABP05-CDR1) | |
| SEQ ID NO: 34 | IDSGGGVT |
| (sdABP05-CDR2) | |
| SEQ ID NO: 35 | VKNYGSTSLQS |
| (sdABP05-CDR3) | |
| SEQ ID NO: 36 | GFTFSNYD |
| (sdABP06-CDR1) | |
| SEQ ID NO: 37 | ISYVGGLT |
| (sdABP06-CDR2) | |
| SEQ ID NO: 38 | ARVKSMHPTSTTGEYDY |
| (sdABP06-CDR3) | |
| SEQ ID NO: 39 | ESTLDNYA |
| (sdABP07-CDR1) | |
| SEQ ID NO: 40 | VGKSGGRS |
| (sdABP07-CDR2) | |
| SEQ ID NO: 41 | AARRVWFGGCVLGTSQGQYDY |
| (sdABP07-CDR3) | |
| SEQ ID NO: 42 | RFTLERYT |
| (sdABP08-CDR1) | |
| SEQ ID NO: 43 | ISSSGGDT |
| (sdABP08-CDR2) | |
| SEQ ID NO: 44 | AARTYACDYKSRWLTYEF |
| (sdABP08-CDR3) | |
| SEQ ID NO: 45 | GFTFSSYG |
| (sdABP09-CDR1) | |
| SEQ ID NO: 46 | ISISGSRT |
| (sdABP09-CDR2) | |
| SEQ ID NO: 47 | RNILV |
| (sdABP09-CDR3) | |

### Example 2. Thermostability (Tₘ) of Human DPEP-1-Specific V_{H}Hs

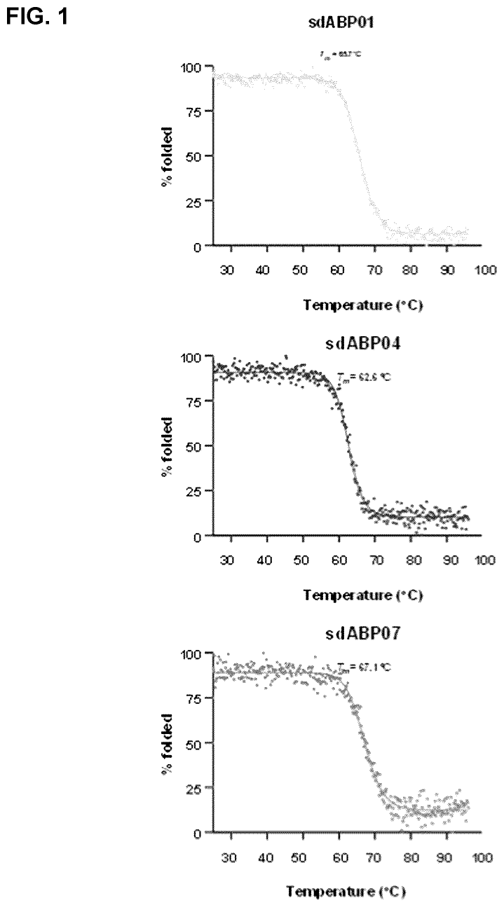

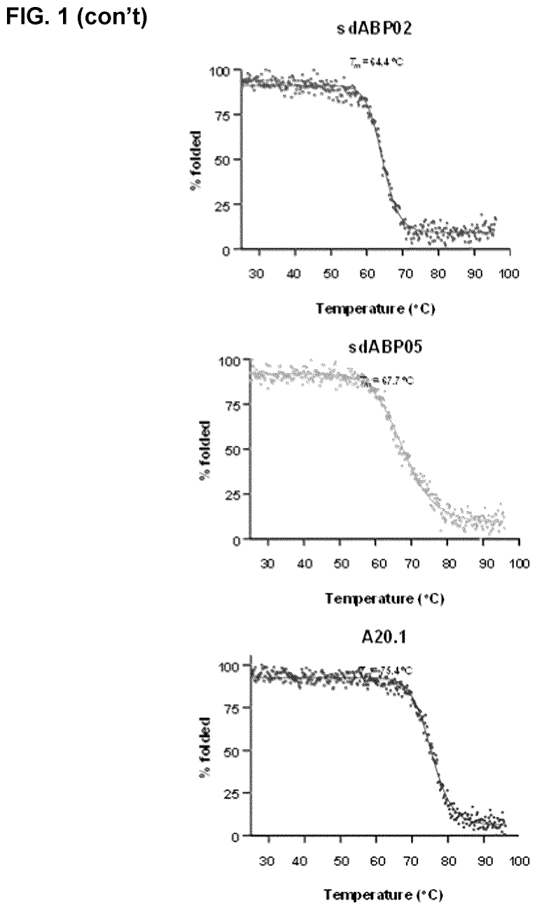

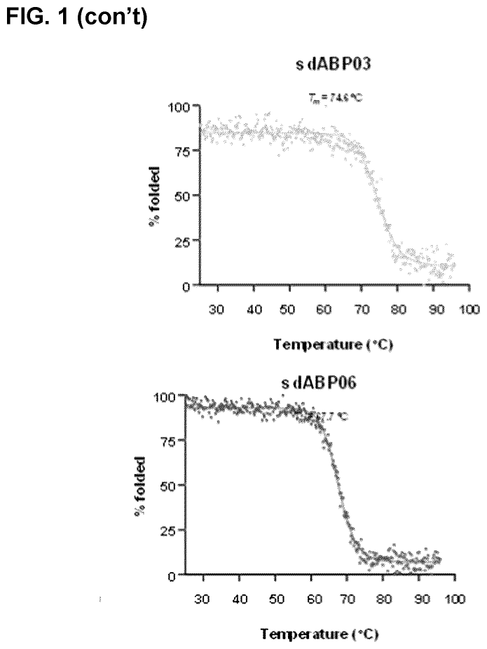

V_{H}H thermal unfolding midpoint temperatures (Tₘs) were determined using circular dichroism spectroscopy by following V_{H}H unfolding at 200 μg/mL concentration and 205 nm wavelength in 100 mM phosphate buffer pH 7.4 (Henry et al., 2017, Front Immunol 8:1759; herein incorporated by reference). Ellipticity measurements were normalized to percentage scale and Tₘs were determined from plot of % folded vs temperature and fitting the data to a Boltzmann distribution. Tₘs (temperatures at the denaturation midpoint) were then determined by Boltzmann curve fitting (FIG. 1). Summary of V_{H}H Tₘs is shown in Table 3. Clostridium difficile toxin A-specific A20.1 V_{H}H (Hussack et al., J Biol Chem., 286:8961-8976 [2011]) was included as reference. These results show that all the tested sdABs have good thermostability, and the thermostability of sdABP03 is particularly good.

TABLE 3
Summary of V_{H}H Tₘs.

| V_{H}H | Tₘ (° C.) |
|---|---|
| sdABP01 | 65.7 |
| sdABP02 | 64.4 |
| sdABP03 | 74.6 |
| sdABP04 | 62.6 |
| sdABP05 | 67.7 |
| sdABP06 | 67.6 |
| sdABP07 | 67.1 |
| A20.1 | 75.4 |

### Example 3. SPR Binding Affinity of Human DPEP-1-Specific V_{H}Hs

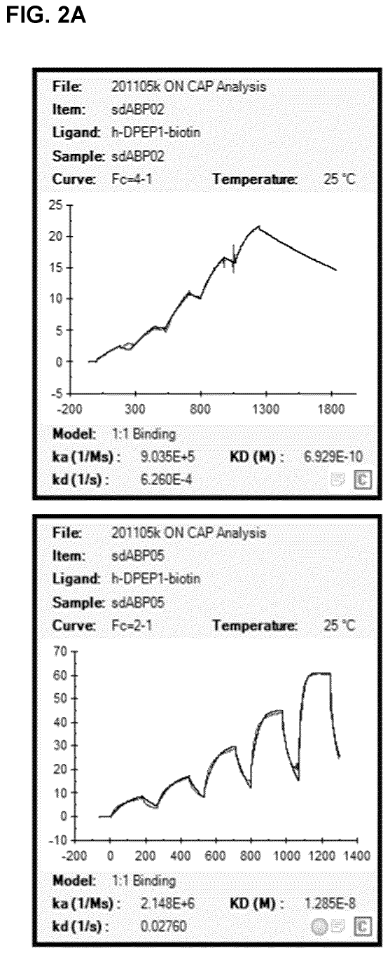

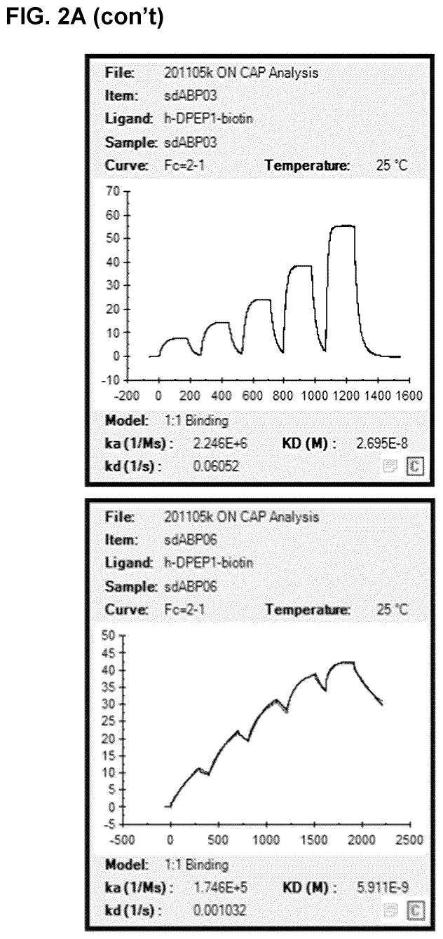

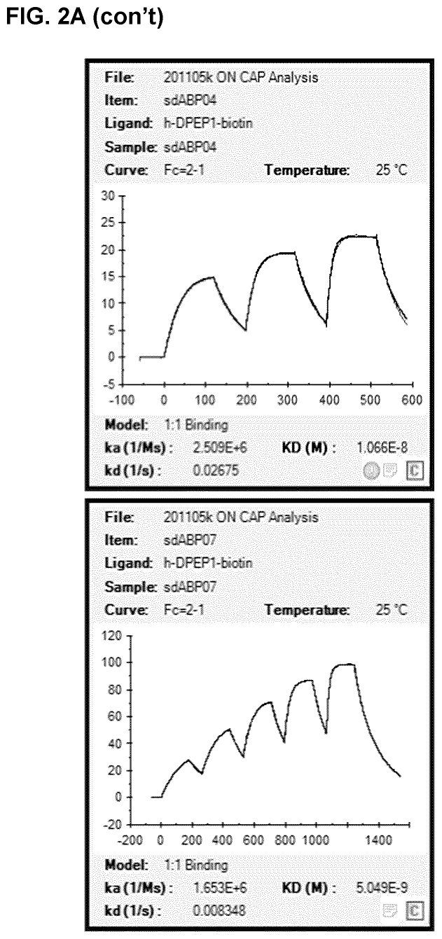

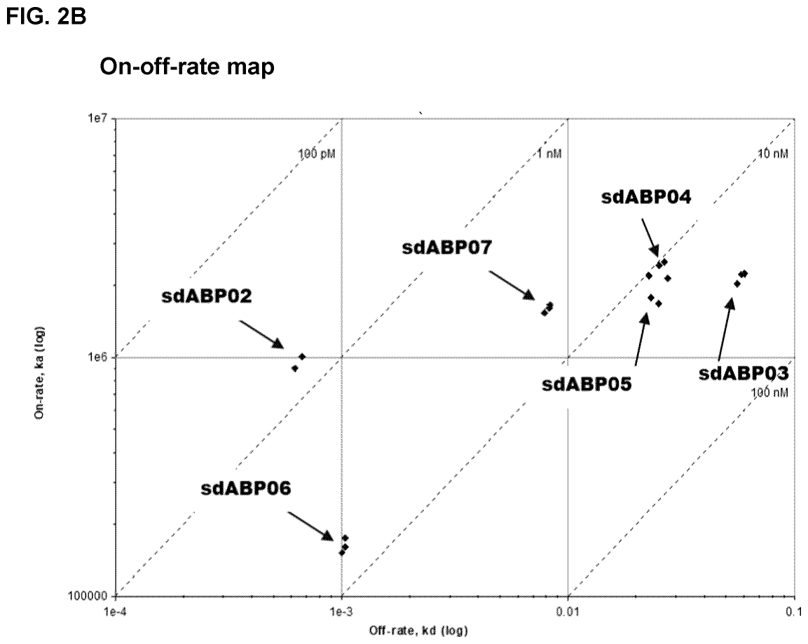

The binding affinity of DPEP-1-specific V_{H}Hs was analyzed by surface plasmon resonance (SPR). Recombinant human DPEP-1 (hDPEP-1) ectodomain was chemically biotinylated using EZ-Link™ NHS-LC-LC-Biotin (Thermo Fisher, Cat #21343) following manufacturer instructions. Biotinylated recombinant hDPEP-1 ectodomain was then captured on CM5 sensorchip surfaces using Biotin CAPture reagent followed by flowing over surfaces V_{H}Hs at concentrations ranging from 0.625-10 nM (sdABP02), 2.5-40 nM (sdABP03/05/07), 6.25-100 nM (sdABP06) and 12.5-200 nM (sdABP01/04). Dark lines represent data points, light lines fit to the data. Data were generated in triplicates. SPR sensorgrams showing single-cycle kinetic analysis of V_{H}Hs binding to human DPEP-1 are shown in FIG. 2A. On-/off-rate maps summarizing V_{H}H kinetic rate constants, kₐs, kₐs, obtained in FIG. 2A are shown in FIG. 2B. Diagonal lines represent equilibrium dissociation constants, K_{D}s. K_{D} values (mean±SD) obtained in FIG. 2A are shown in Table 4. sdABP01 binding data gave poor fit to the 1:1 binding model. As a result, its K_{D} could not be determined with certainty. A better fit was observed using the heterogeneous ligand binding model. Affinities (K_{D}s) derived from the heterogeneous binding model were approximately 30 nM, which is in general agreement with affinities obtained using the more traditional 1:1 binding model fits.

TABLE 4
Summary of K_{D} values from FIG. 2A.

| V_{H}H | K_{D} (nM) |
|---|---|
| sdABP01 | Not available |
| sdABP02 | 0.68 ± 0.01 |
| sdABP03 | 27.03 ± 0.42 |
| sdABP04 | 10.50 ± 0.14 |
| sdABP05 | 10.63 ± 0.97 |
| sdABP06 | 6.32 ± 0.31 |
| sdABP07 | 5.09 ± 0.03 |

### Example 4: Binding of DPEP-1-Specific V_{H}Hs to Cell-Displayed Human DPEP-1

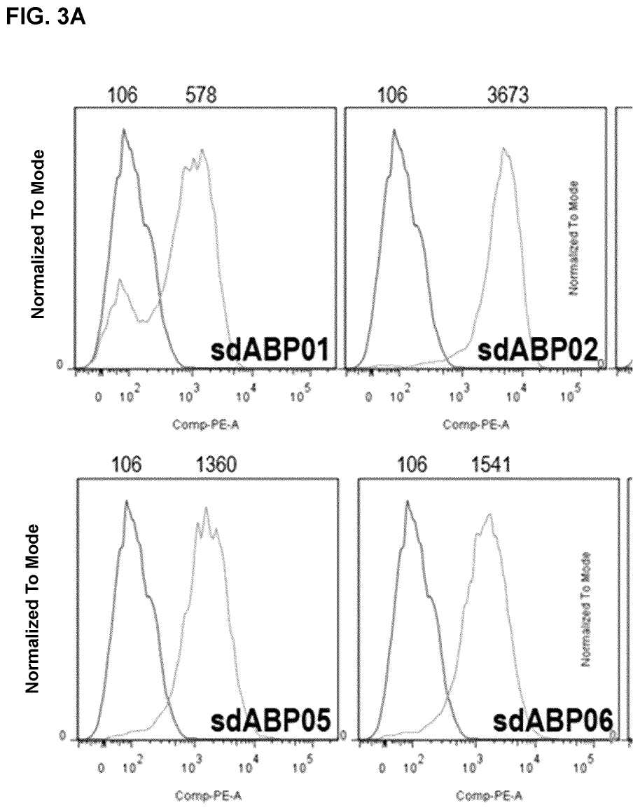

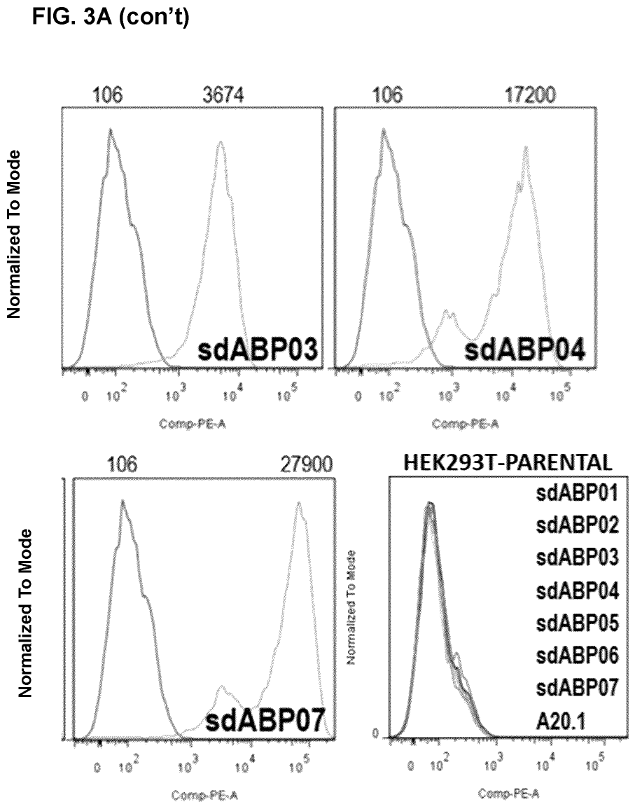

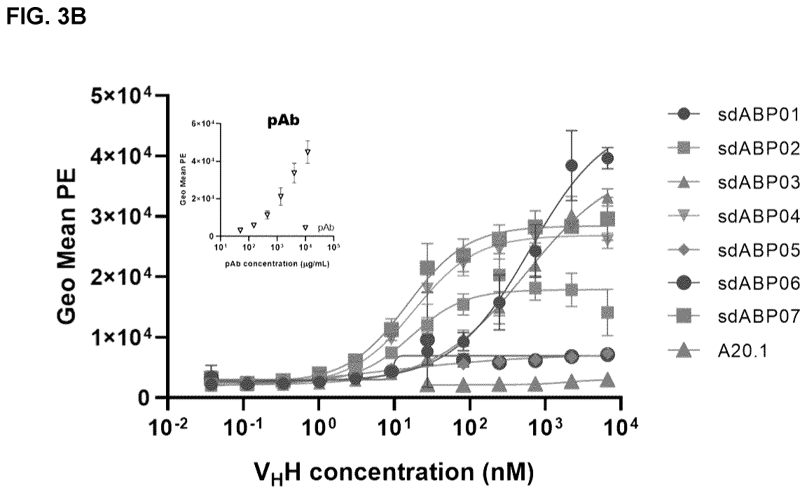

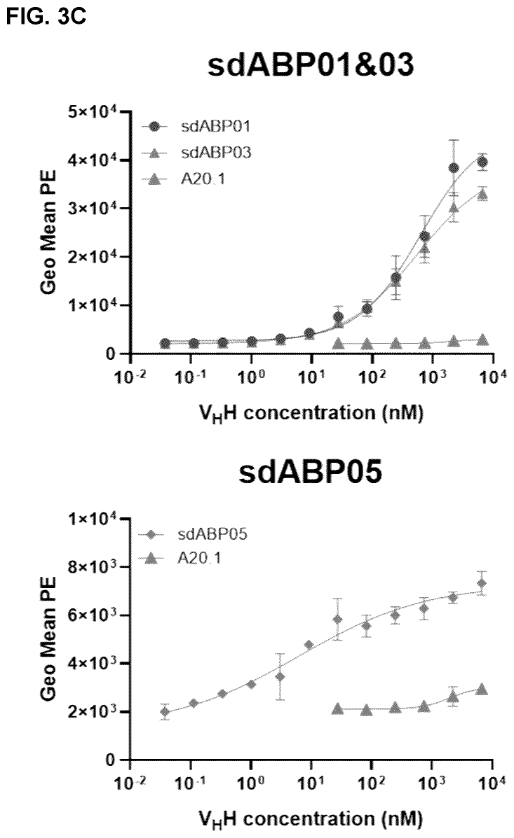

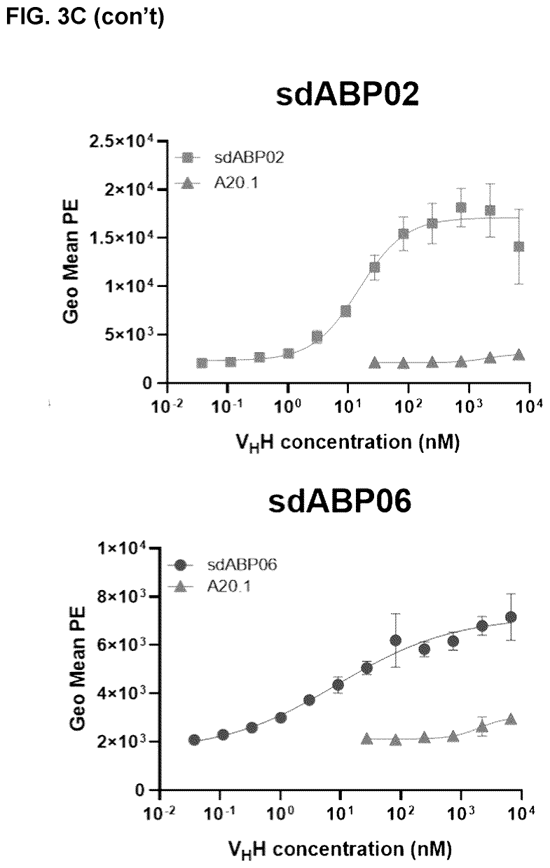

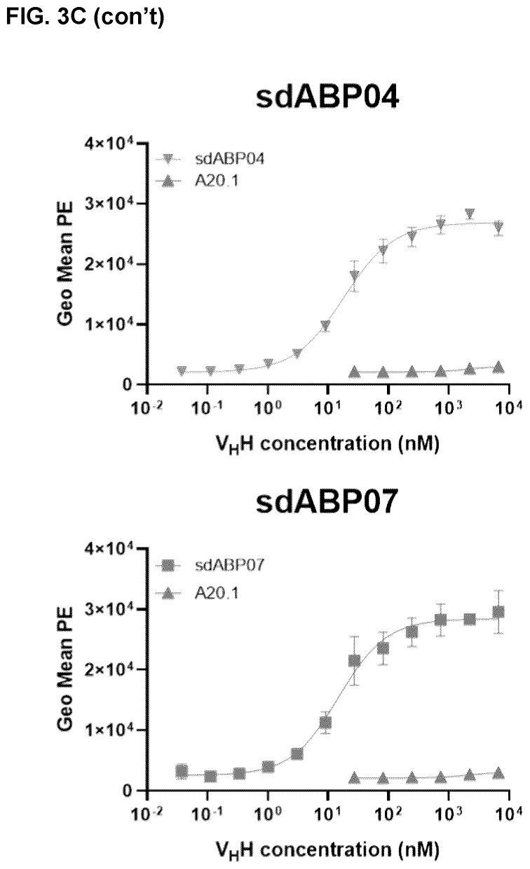

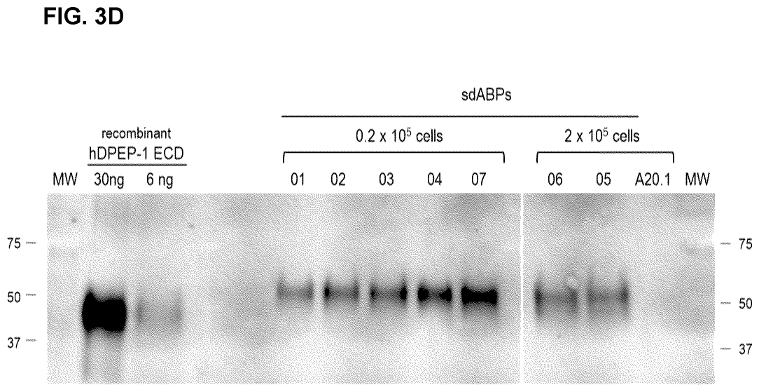

Binding of DPEP-1 specific V_{H}Hs against human DPEP-1 was evaluated by flow cytometry. Transfected HEK293T cells overexpressing full length human DPEP-1 were used as targets and parental HEK293T cells which lack DPEP-1 expression were used as a control. Each cell line was detached using Accutase® solution, washed and then 2×10⁵ cells were incubated for one hour at 4° C., with 100 μL of biotinylated sdAb01-07 (at a final concentration of 100 nM). Binding was detected using streptavidin-phycoerythrin (SPE, Thermo Fisher, Cat #S866). Positive binding of HEK-293T-hDPEP1+ are the profiles on the right side in each graph as shown in FIG. 3A. Clostridium difficile toxin A-specific A20.1 V_{H}H (Hussack et al., J Biol Chem., 286: 8961-8976 [2011]) was included as negative V_{H}H control (profiles on the left side in each graph). Spectral shift to the right as seen in the case of DPEP-1-specific V_{H}Hs are indicative of binding to human DPEP-1 and quantified as increase in geometric mean fluorescence intensity (recorded above histograms). No binding was seen when indicated V_{H}Hs were tested against HEK293T-PARENTAL which is negative for hDPEP-1 expression (i.e. no spectral shift to the right). V_{H}H binding was detected using phycoerythrin-labeled streptavidin. Data were collected on a FACScalibur (BD Bioscience), followed by analysis with FlowJo v10.6.2 (TreeStar). These results show that all seven sdABs tested were able to recognize cell-displayed DPEP-1. No binding was observed against parental HEK293T cells which do not express DPEP-1 even at 1000 nM. Together, these results show that sdABs identified in Example 1 are specific to human DPEP-1.

### Example 5. Dose Response Binding of Human DPEP-1-Specific V_{H}Hs to Cell-Displayed Human DPEP-1

Dose response binding of human DPEP-1-specific V_{H}Hs was analyzed against cell-displayed human DPEP-1 (hDPEP-1). Flow cytometry analysis of biotinylated V_{H}Hs was performed at increasing V_{H}H concentrations against HEK-293-6E cells overexpressing (hDPEP-1) (see FIG. 3B). Toxin A-specific A20.1 V_{H}H (Hussack et al., J Biol Chem., 286:8961-8976, (2011)) was included as negative V_{H}H control. Binding of biotinylated V_{H}Hs to hDPEP-1 was detected using streptavidin-phycoerythrin (SPE). The binding profile of the anti-DPEP-1 rabbit pAb (Proteintech, Cat #12222-1-AP) positive control is shown as inset. Binding of the rabbit pAb was detected using goat anti-rabbit IgG antibody conjugated to phycoerythrin (Thermo Fisher, Cat #P-2771MP). Graphs in FIG. 3B with similar maximal fluorescence plateau are grouped together and shown in FIG. 3C (A20.1 V_{H}H is shown for comparison). Three independent experiments were performed and data were collected on a Beckman CytoFLEX Analyzer followed by analysis with FlowJo v10.6.2 (TreeStar). EC₅₀ values were calculated from FIG. 3C and recorded in Table 5. Based on the binding measured by flow cytometry, four affinity groups may be identified: medium (sdABP01/03), high (sdABP02/04/07), high-very high (sdABP05), and very high (sdABP06). These results confirm the binding detected by SPR and also show that these human DPEP-1-specific V_{H}Hs bind to hDPEP-1-overexpressing cells.

Next, hDPEP-1 from HEK293-6E cells overexpressing full length hDPEP-1 were shown to be immunoprecipitated by V_{H}Hs described in this disclosure. Individual biotinylated V_{H}Hs were captured on neutravidin-sepharose beads (Thermo Fisher, Cat #29202) and were subsequently incubated with Triton X-100-solubilized HEK293T-DPEP-1+ cells. The pulled-down proteins were then separated on SDS-PAGE gel, transferred to PVDF, probed with anti-DPEP-1 rabbit pAb and detected with goat anti-rabbit:HRP (Jackson Immunoresearch, Cat #111-035-144) using SuperSignal™ West Pico PLUS Chemiluminescent Substrate (Thermo Fisher, Cat #34578). Pure recombinant hDPEP-1 ectodomain (30 and 6 ng/well) was included as positive control in assays. The immunoblotting results are shown in FIG. 3D. These results confirm the binding and show that the V_{H}Hs bind specifically to human DPEP-1 on the surface of cells.

TABLE 5
Summary of EC₅₀ values from FIG. 3C.

| V_{H}H | EC₅₀ (nM) |
|---|---|
| sdABP01 | 630 |
| sdABP02 | 17 |
| sdABP03 | 550 |
| sdABP04 | 18 |
| sdABP05 | 5.3 |
| sdABP06 | 0.96 |
| sdABP07 | 15 |

### Example 6. Epitope Binning of sdABPs by SPR and ELISA

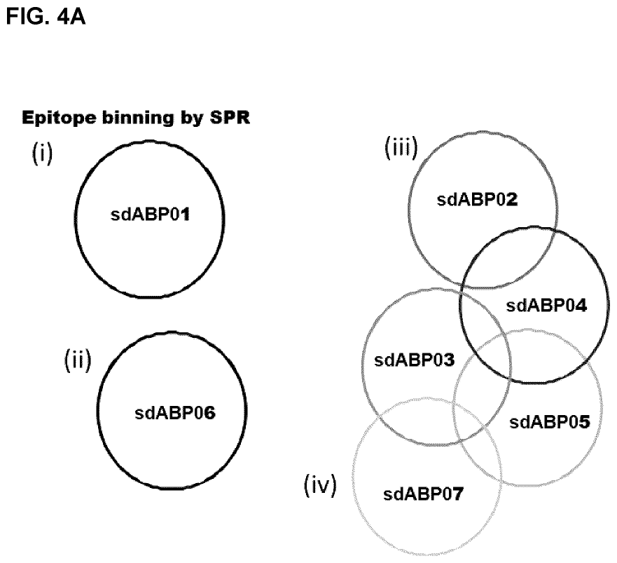

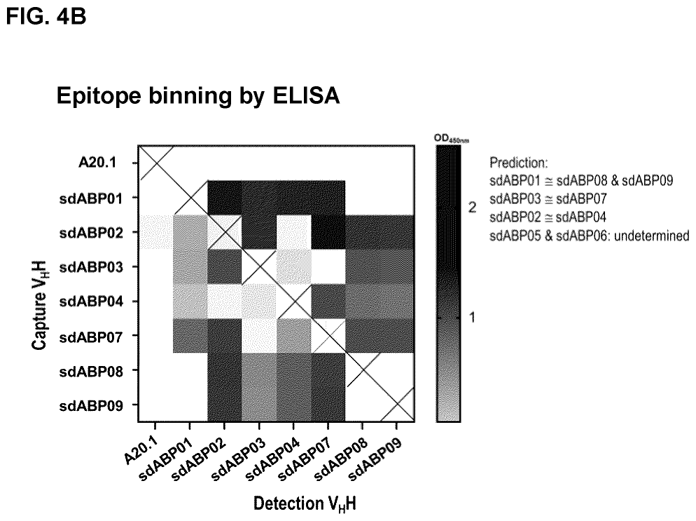

Epitope bins identified by SPR are summarized in FIG. 4A. sdABP01, sdABP06, sdABP02 and sdABP07 defined bins (i), (ii), (iii) and (iv), respectively. sdABP03 and sdABP05 partially overlap bin (iv), while sdABP04 partially overlaps bin (iii). Epitope binning of sdABPs by ELISA were as described in Rossotti 2015 et al (“Streamlined method for parallel identification of single domain antibodies to membrane receptors on whole cells”, Biochim Biophys Acta, 1850(7): 1397-404; herein incorporated by reference). FIG. 4B are schematics of the competitive sandwich ELISA performed to cluster V_{H}Hs by epitopes and represented as a heat map displaying all possible pair-wise combinations of V_{H}Hs (7×7=49). Binding pairs showing high binding signal (dark) were considered as recognizing non-overlapping epitopes hence belonging to different epitope bins or V_{H}H clusters, while those giving no or weak binding signals (colorless/light) were considered recognizing overlapping epitopes, thus belonging to the same epitope bins. A20.1 V_{H}H, specific for Clostridium difficile toxin A, included as negative control (Hussack et al., 2011) did not give any binding signals. Hence, sdABP02, sdABP03, sdABP04, sdABP05, and sdABP07 appear to all clustered around one primary epitope. The ELISA epitope binning results confirmed those of SPR results and further showed sdABP08 and sdABP09 belonged to the same bin (i) as sdABP01.

### Example 7. Human DPEP-1-Specific sdABP Reduced LPS-Induced Renal Inflammation

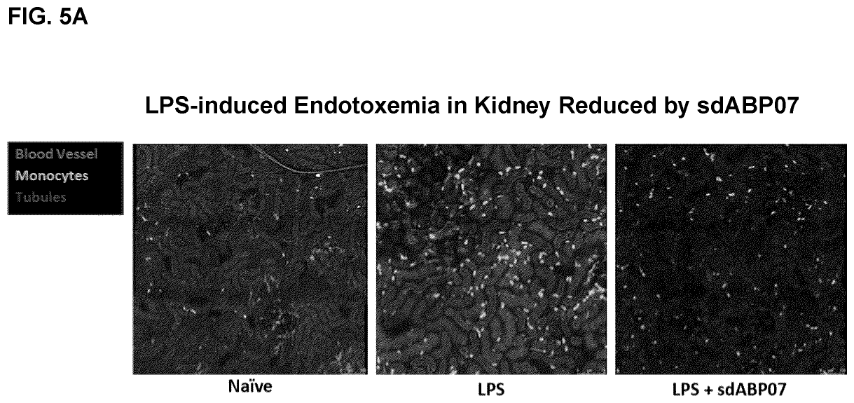

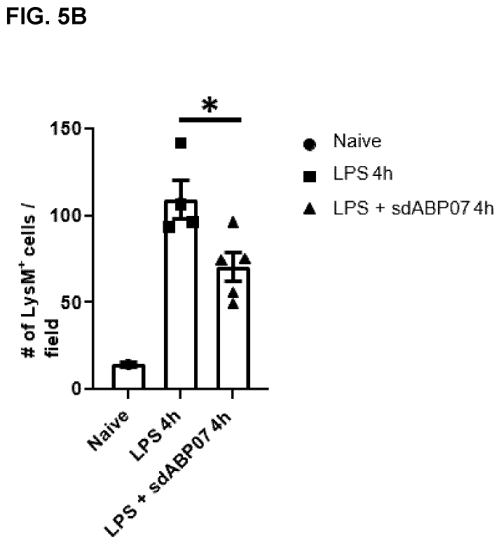

Since sdABP07 shows promising and selective binding against human DPEP-1 under immunofluorescent staining, as well as showing high affinity binding to human DPEP-1 by flow cytometry (FIG. 3C and Table 5), this antibody was selected for further testing in its ability to affect LPS-induced endotoxemia. LysM^{gfp/gfr} mice were treated with LPS (5 mg/kg, IV) to induce endotoxemia in the presence or absence of sdABP07 (50 μg). The kidneys of the animals were imaged live by intravital microscopy at 4 hours post-treatment. The immunofluorescent images of FIG. 5A show that sdABP07 reduced monocytes infiltration in kidneys of LysM^{gfp/gfr} mice treated with LPS (right panel) as compared with mice treated with LPS alone (middle panel), indicative of sdABP07 reducing LPS-induced renal inflammation. Renal inflammation was quantified by the number of adhered LysM⁺ monocytes found per field (n=4-5/group, *: p<0.05) in the kidneys of these mice (see FIG. 5B).

### Example 8. Production of Anti-DPEP-1 V_{H}Hs Fused to Human IgG1 Hinge-Fc (V_{H}H-Fcs) in Mammalian Cells

Codon-optimized genes for bivalent V_{H}H-Fcs were synthesized (GeneArt, Thermo Fisher) and cloned into pTT5-hIgGIFc between the genes for human V_{H} leader sequence and the human IgG1 hinge/Fc sequences. V_{H}H-Fcs (SEQ ID NOS: 48-56) were produced by transient expression in HEK293-6E cells and purified by protein A affinity chromatography from culture supernatants as describe in Durocher, Y. et al, “High-level and high-throughput recombinant protein production by transient transfection of suspension-growing human 293-EBNAI cells” Nucleic Acids Res 30, E9 (2002) and Rossotti, M. A. et al. “Camelid single-domain antibodies raised by DNA immunization are potent inhibitors of EGFR signaling” Biochem J 476, 39-50 (2019) (herein incorporated by reference in each of its entirety). Purified V_{H}H-Fcs were buffer exchanged against phosphate-buffered saline (PBS), pH 7.4 using Amicon® Ultra-15 Centrifugal Filter Units (Millipore, Cat #UFC905024). The purity of DPEP-1 V_{H}H-Fcs was confirmed by SDS-PAGE using 4-20% Mini-PROTEAN R TGX Stain-Free™ Gels (Biorad. Cat #17000435). Table 6 lists the sequences of the nine expressed DPEP-1 V_{H}H-Fcs.

TABLE 6
Amino acid sequences of VHH-Fcs.

| SEQ ID NO | Amino Acid Sequence |
|---|---|
| SEQ ID NO: 48 | EVQLVESGGGLVQPGGSLRLSCAASGSTLNWYTIGWFRQAPGKEREEVSCISSSGGSTKYA |
| (sdABP01-Fc) | DSVKGRFTISRSNALNTVYLQMNTLKPDDTAVYYCALDLDSAFCGSHISEYEYWGQGTQVT |
| | VSSAEPKSCDKTHTCPPCPAPELLGGPSVFLFPPKPKDTLMISRTPEVTCVVVDVSHEDPE |
| | VKFNWYVDGVEVHNAKTKPREEQYNSTYRVVSVLTVLHQDWLNGKEYKCKVSNKALPAPIE |
| | KTISKAKGQPREPQVYTLPPSRDELTKNQVSLTCLVKGFYPSDIAVEWESNGQPENNYKTT |
| | PPVLDSDGSFFLYSKLTVDKSRWQQGNVESCSVMHEALHNHYTQKSLSLSPG |
| SEQ ID NO: 49 | EVQLVESGGGSVQAGGSLRLSCVASGIHFGSHSMAWYRQAPGKERDLVARISALGNTNYAN |
| (sdABP02-Fc) | SVKGRFTISRDTNKSTLYLQMNTLKPEDTAMYYCAPWSAYDREGDERSWGQGTQVTVSSAE |
| | PKSCDKTHTCPPCPAPELLGGPSVFLFPPKPKDTLMISRTPEVTCVVVDVSHEDPEVKENW |
| | YVDGVEVHNAKTKPREEQYNSTYRVVSVLTVLHQDWLNGKEYKCKVSNKALPAPIEKTISK |
| | AKGQPREPQVYTLPPSRDELTKNQVSLTCLVKGFYPSDIAVEWESNGQPENNYKTTPPVLD |
| | SDGSFFLYSKLTVDKSRWQQGNVFSCSVMHEALHNHYTQKSLSLSPG |
| SEQ ID NO: 50 | EVQLVESGGGLVQPGGSLRLSCATSEFTLDYYAIGWFRQAPGKEREGVSCISSSGGTTNYA |
| (sdABP03-Fc) | DSVKGRFTISSDNAKNTVSLQMNSLRPEDTAVYYCAAARVSAYYLGNYGCLNAEYGYWGQG |
| | TQVTVSSAEPKSCDKTHTCPPCPAPELLGGPSVFLFPPKPKDTLMISRTPEVTCVVVDVSH |
| | EDPEVKENWYVDGVEVHNAKTKPREEQYNSTYRVVSVLTVLHQDWLNGKEYKCKVSNKALP |
| | APIEKTISKAKGQPREPQVYTLPPSRDELTKNQVSLTCLVKGFYPSDIAVEWESNGQPENN |
| | YKTTPPVLDSDGSFFLYSKLTVDKSRWQQGNVFSCSVMHEALHNHYTQKSLSLSPG |
| SEQ ID NO: 51 | EVQLVESGGGLVQPGGSLRLSCAASGFTESSYYMSWVRQAPGKGPEWVSGINTDGDDTSYA |
| (sdABP04-Fc) | DSVKGRFTISRDNAKNTLYLQMSSLKPEDTALYYCARAARSGSTTWGRNYWGQGTQVTVSS |
| | AEPKSCDKTHTCPPCPAPELLGGPSVFLFPPKPKDTLMISRTPEVTCVVVDVSHEDPEVKE |
| | NWYVDGVEVHNAKTKPREEQYNSTYRVVSVLTVLHQDWLNGKEYKCKVSNKALPAPIEKTI |
| | SKAKGQPREPQVYTLPPSRDELTKNQVSLTCLVKGFYPSDIAVEWESNGQPENNYKTTPPV |
| | LDSDGSFFLYSKLTVDKSRWQQGNVFSCSVMHEALHNHYTQKSLSLSPG |
| SEQ ID NO: 52 | EVQLVESGGGLVQPGGSLRLSCAASGFTFSTYAMTWVRKAPGKGFEWISSIDSGGGVTLYA |
| (sdABP05-Fc) | DSVKGRFTISKDNAKNTLYLQMNNLKPDDTAVYYCVKNYGSTSLQSRGQGTQVTVSSAEPK |
| | SCDKTHTCPPCPAPELLGGPSVFLFPPKPKDTLMISRTPEVTCVVVDVSHEDPEVKENWYV |
| | DGVEVHNAKTKPREEQYNSTYRVVSVLTVLHQDWLNGKEYKCKVSNKALPAPIEKTISKAK |
| | GQPREPQVYTLPPSRDELTKNQVSLTCLVKGFYPSDIAVEWESNGQPENNYKTTPPVLDSD |
| | GSFFLYSKLTVDKSRWQQGNVFSCSVMHEALHNHYTQKSLSLSPG |
| SEQ ID NO: 53 | EVQLVDSGGGLVQPGGSLRLSCAASGFTFSNYDMSWVRQGPGKGPEWVSIISYVGGLTRYS |
| (sdABP06-Fc) | DSVKGRFTISRDNAKNTLYLQMNSLNTEDTALYYCARVKSMHPTSTTGEYDYRGRGTQVTV |
| | SSAEPKSCDKTHTCPPCPAPELLGGPSVFLFPPKPKDTLMISRTPEVTCVVVDVSHEDPEV |
| | KFNWYVDGVEVHNAKTKPREEQYNSTYRVVSVLTVLHQDWLNGKEYKCKVSNKALPAPIEK |
| | TISKAKGQPREPQVYTLPPSRDELTKNQVSLTCLVKGFYPSDIAVEWESNGQPENNYKTTP |
| | PVLDSDGSFFLYSKLTVDKSRWQQGNVESCSVMHEALHNHYTQKSLSLSPG |
| SEQ ID NO: 54 | EVQLVESGGGLVEYGGSLRLSCAASESTLDNYAIAWERQAPGKEREVVSCVGKSGGRSDYA |
| (sdABP07-Fc) | DSVKGRFTISRDNAKNTVYLQMNSLKPEDTAVYSCAARRVWFGGCVLGTSQGQYDYWGQGT |
| | QVTVSSAEPKSCDKTHTCPPCPAPELLGGPSVFLFPPKPKDTLMISRTPEVTCVVVDVSHE |
| | DPEVKFNWYVDGVEVHNAKTKPREEQYNSTYRVVSVLTVLHQDWLNGKEYKCKVSNKALPA |
| | PIEKTISKAKGQPREPQVYTLPPSRDELTKNQVSLTCLVKGFYPSDIAVEWESNGQPENNY |
| | KTTPPVLDSDGSFFLYSKLTVDKSRWQQGNVFSCSVMHEALHNHYTQKSLSLSPG |
| SEQ ID NO: 55 | EVQLVSSGGGLVQPGGSLRLSCKASRFTLERYTIGWERQAPGKEREGIACISSSGGDTNYA |
| (sdABP08-Fc) | DSVKGRFTISRDNVVEKVYLQMDSLKPEDTAVYYCAARTYACDYKSRWLTYEFRGQGTQVT |
| | VSSAEPKSCDKTHTCPPCPAPELLGGPSVFLFPPKPKDTLMISRTPEVTCVVVDVSHEDPE |
| | VKFNWYVDGVEVHNAKTKPREEQYNSTYRVVSVLTVLHQDWLNGKEYKCKVSNKALPAPIE |
| | KTISKAKGQPREPQVYTLPPSRDELTKNQVSLTCLVKGFYPSDIAVEWESNGQPENNYKTT |
| | PPVLDSDGSFFLYSKLTVDKSRWQQGNVFSCSVMHEALHNHYTQKSLSLSPG |
| SEQ ID NO: 56 | QVQLVESGGGLVQPGGSLRLSCAASGFTFSSYGMTWVRQAPGKGFEWVSTISISGSRTTYA |
| (sdABP09-Fc) | GSVKDRFTISRDNAKNTLYLQMNSLKPEDTAVYYCRNILVQGQGTQVTVSSAEPKSCDKTH |
| | TCPPCPAPELLGGPSVFLFPPKPKDTLMISRTPEVTCVVVDVSHEDPEVKENWYVDGVEVH |
| | NAKTKPREEQYNSTYRVVSVLTVLHQDWLNGKEYKCKVSNKALPAPIEKTISKAKGQPREP |
| | QVYTLPPSRDELTKNQVSLTCLVKGFYPSDIAVEWESNGQPENNYKTTPPVLDSDGSFFLY |
| | SKLTVDKSRWQQGNVFSCSVMHEALHNHYTQKSLSLSPG |

### Example 9. Size Exclusion Chromatography Analysis of DPEP-1 V_{H}H-Fcs

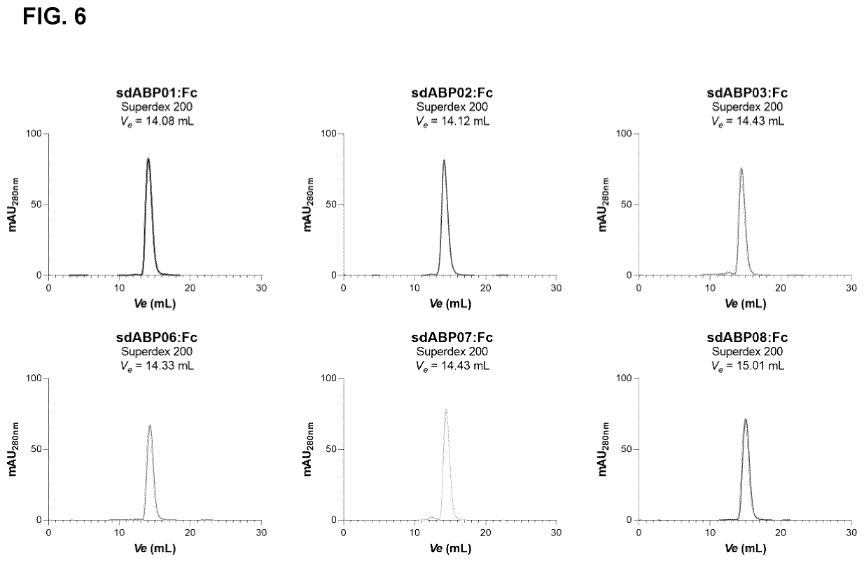

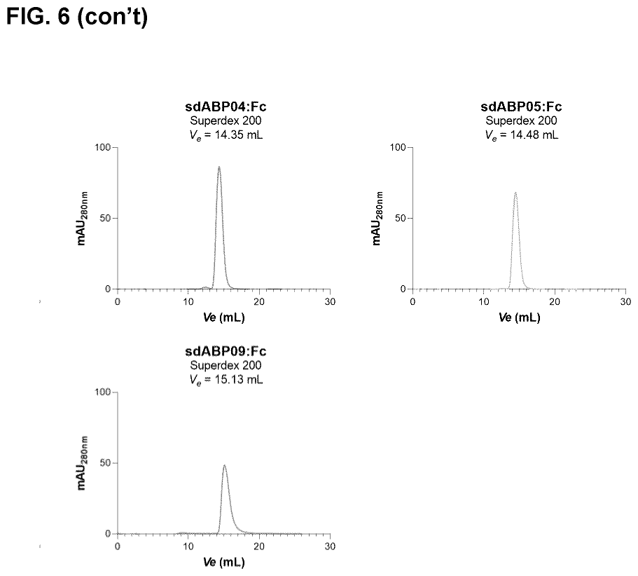

Purified V_{H}Hs-Fcs were subjected to size-exclusion chromatography (SEC) to assess their aggregation resistance. 150 μg of each V_{H}H-Fc was injected into Superdex™ 200 Increase 10/300 GL column (Cytiva) connected to an ÄKTA FPLC protein purification system (Cytiva) as previously described in Hussack, G. et al., “Neutralization of Clostridium difficile toxin A with single-domain antibodies targeting the cell receptor binding domain”. J Biol Chem 286, 8961-8976 (2011), herein incorporated by reference. PBS was used as running buffer at 0.8 mL/min. Similar to their V_{H}H counterparts, all V_{H}H-Fcs were free of any aggregates (FIG. 6).

### Example 10. Binding Validation of DPEP-1 V_{H}H-Fcs by ELISA

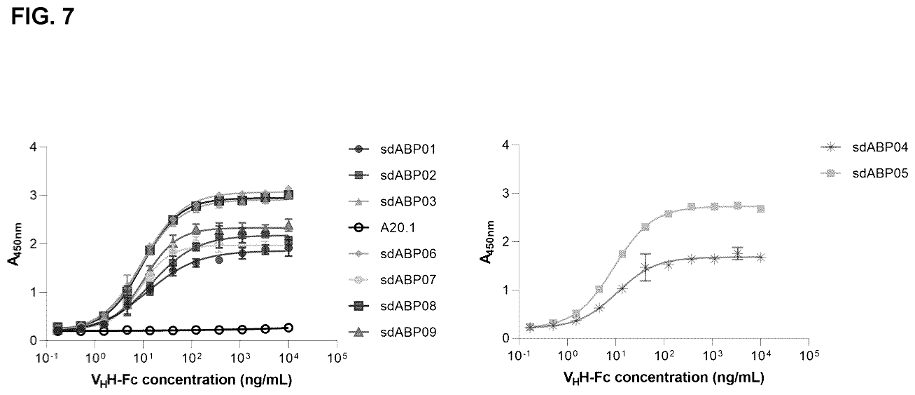

The binding of V_{H}H-Fcs to DPEP-1 was assessed by ELISA. Microtiter well plates were coated with 50 ng/well of the recombinant human DPEP-1 ectodomain (SinoBiologicals, Cat #13543-H08H) in 100 μL PBS overnight at 4° C. Plates were blocked with PBSC (1% casein [w/v]) in PBS; Thermo Fisher, Cat #37528) for 1 h at room temperature, then washed five times with PBST (PBS supplemented with 0.1% (v/v) Tween 20; Thermo Fisher, Cat #28352) and incubated with varying concentrations of V_{H}Hs-Fcs. After 1 h incubation, plates were washed 10 times with PBST and binding of V_{H}Hs-Fc was probed for 1 h with 1 μg/mL of HRP-conjugated goat anti-human IgG (SIGMA, Cat #A0170). Plates were washed 10 times and incubated with 100 μL peroxidase substrate solution (SeraCare, Cat #50-76-00) at room temperature for 15 min. Reactions were stopped by adding 50 μL 1 M H₂SO₄ to wells, and absorbance were subsequently measured at 450 nm using a Multiskan™FC photometer (Thermo Fisher). Data were fitted to non-linear regression ([Agonist] vs. response, Variable slope (four parameters)) using GraphPad Prism version 9 (La Jolla, CA). FIG. 7 demonstrates all nine V_{H}H-Fcs bound to their antigen, human DPEP-1.

### Example 11. Assessing Binding Affinity, Specificity and Cross-Reactivity of DPEP-1 V_{H}H-Fcs by Flow Cytometry

HEK293-6E cells were grown in Gibco™ FreeStyle™ F17 Expression Medium (Thermo Fisher, Cat #A1383501) until they reached a density of 1.5×10⁶ cells/mL. Transient transfection of HEK293-6E cells were carried out with 100 μg pcDNA3.1 expression plasmid encoding full length human DPEP-1, mouse DPEP-1, rat DPEP-1 or human DPEP-2 combined with 100 μg of PEIpro®, DNA transfection reagent (VWR, Cat #71002-812). Cell surface expression of DPEP-1 or DPEP-2 (DPEP-1/2) were carried out for 96 h. To assess cell binding of V_{H}H-Fcs by flow cytometry, DPEP-1/2 expressing cells were harvested and washed once by PBS centrifugation, and resuspended at 1× 10⁶ cells/mL in PBSB (PBS containing 1% [w/v] BSA) and 0.05% [w/v] sodium azide [SIGMA, Cat #S2002]). Fifty μL of individual DPEP-expressing cells were incubated with 50 μL of V_{H}H-Fcs (250 nM) for 1 h on ice. Subsequently, cells were washed twice with PBSB by centrifugation for 5 min at 1200 rpm, and then incubated for an additional hour on ice with 50 μL of R-Phycoerythrin-conjugated AffiniPure Fab Fragment Goat Anti-Human IgG, Fc Fragment Specific (Jackson Immunoresearch, Cat #109-117-008) at 2 μg/mL diluted in PBSB. After a final wash, cells were resuspended in 100 μL PBSB and data were acquired on a CytoFLEX S flow cytometer (Beckman Coulter) and analyzed by FlowJo software (FlowJo LLC, v10.6.2, Ashland). The binding of the anti-human DPEP-1 rabbit pAb (Proteintech, Cat #12222-1-AP) positive control was detected using goat anti-rabbit IgG antibody conjugated to R-phycoerythrin (Thermo Fisher, Cat #P-2771MP).

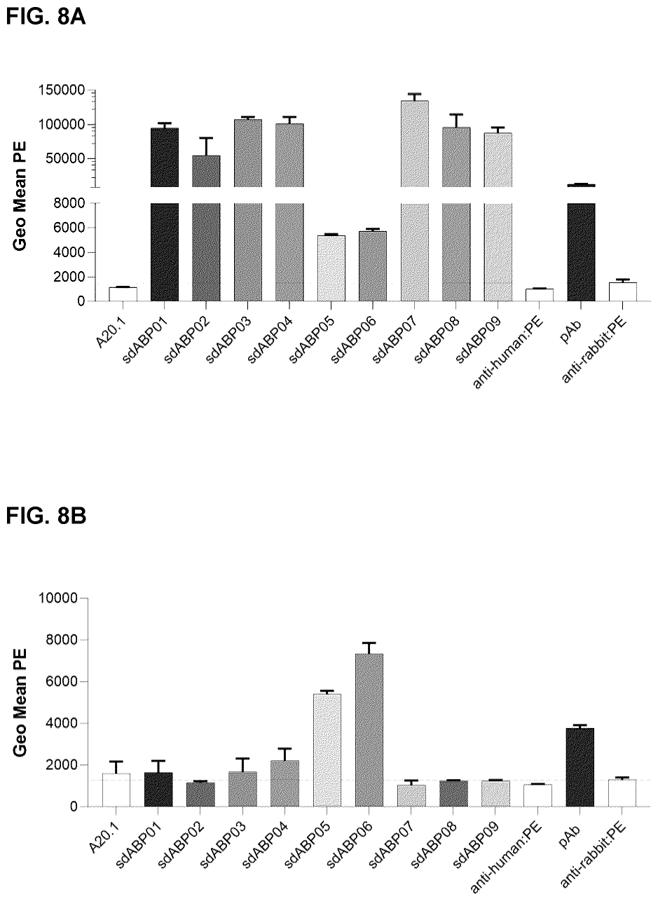

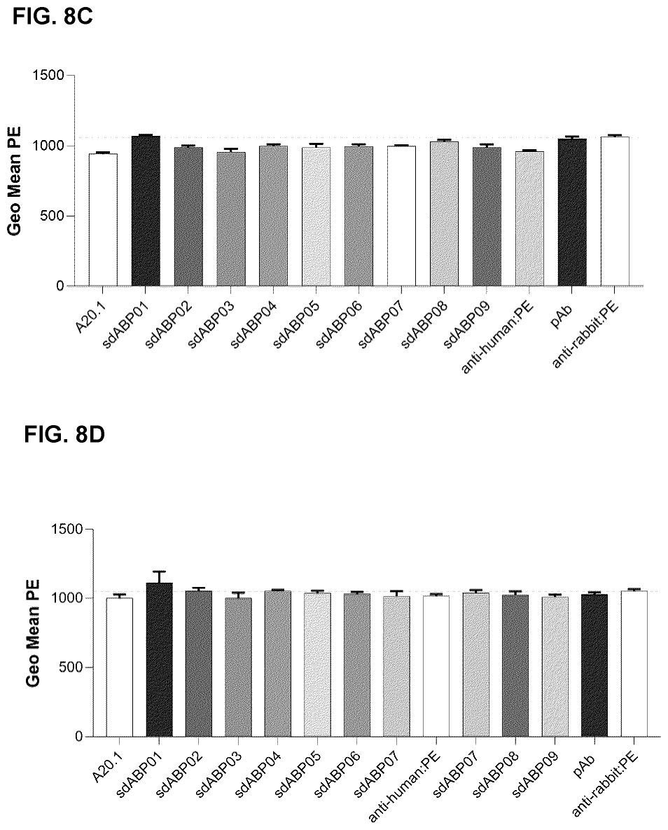

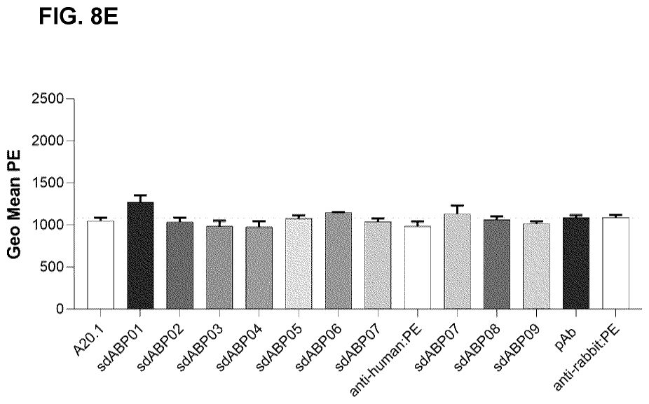

Flow cytometry results performed at fixed V_{H}H-Fc concentrations showed all nine V_{H}H-Fcs bound to DPEP-1-expressing HEK293-6E cells and not to the parental, non-DPEP-1-expressing cells, clearly demonstrating V_{H}H-Fcs are specifically targeting DPEP-1 on the surface of cells (FIG. 8A and FIG. 8E). Flow cytometry experiments were extended to include mouse DPEP-1-, rat DPEP-1- and human DPEP-2-expressing HEK293-6E cells. Results showed the high specificity of V_{H}H-Fcs as none of the V_{H}H-Fcs bound to rat DPEP-1 or human DPEP-2 and only two out of the nine V_{H}H-Fcs (sdABP05 and sdABP06) cross-reacted with mouse DPEP-1 (FIG. 8B, FIG. 8C, and FIG. 8D).

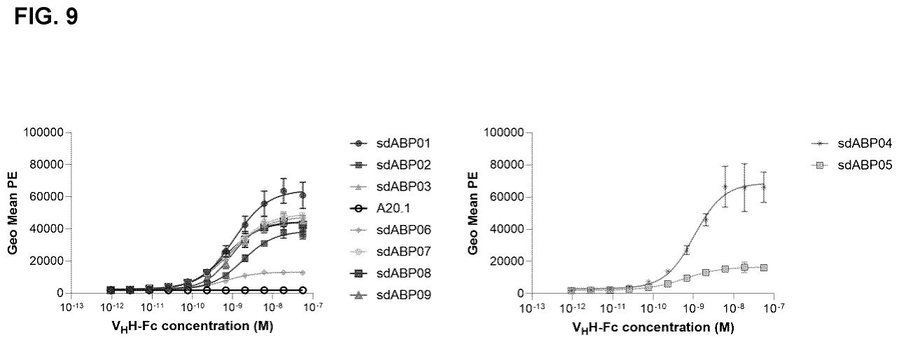

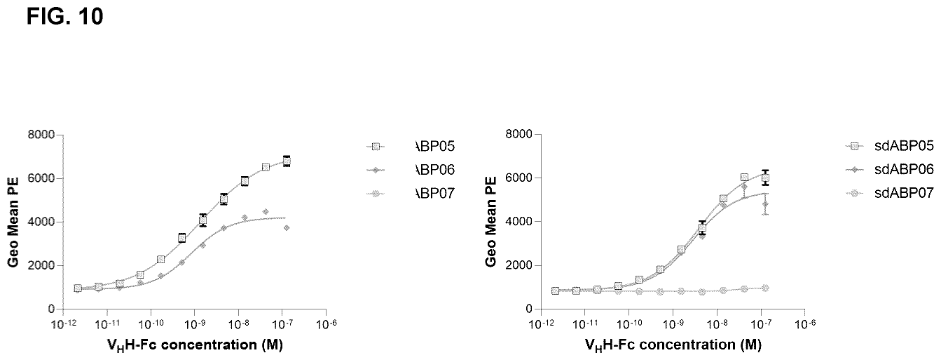

Flow cytometry experiments performed with variable concentrations of V_{H}H-Fcs against human and mouse DPEP-1-expressing HEK293-6E cells allowed determination of apparent EC₅₀s. Affinities against human DPEP-1-expressing HEK293-6E cells were determined to be high (low EC₅₀s; range: 0.6-1.8 nM; median: 0.9 nM) (FIG. 9; Table 7). Comparison of the EC₅₀s of V_{H}Hs (Table 5) to their V_{H}H-Fc counterparts (Table 7) revealed drastic increases in potencies (EC₅₀s) of V_{H}Hs up to 600-fold as a result of Fc-mediated dimerization (see Table 8). Moreover, sdABP05 and sdABP06 were determined to cross-react with mouse DPEP-1 with similar high affinities to human DPEP-1 (FIG. 10; Table 9).

TABLE 7
Summary of apparent EC₅₀ values from FIG. 9.

| V_{H}H-FC | EC₅₀ (nM) |
|---|---|
| sdABP01 | 1.1 |
| sdABP02 | 1.8 |
| sdABP03 | 0.9 |
| sdABP04 | 1.1 |
| sdABP05 | 0.6 |
| sdABP06 | 0.6 |
| sdABP07 | 0.9 |
| sdABP08 | 0.7 |
| sdABP09 | 0.9 |

TABLE 8
Comparison of the EC₅₀s of V_{H}Hs (Table 5)
to their V_{H}H-Fc counterparts (Table 7).

| | EC₅₀ (nM) | | Fold |
|---|---|---|---|
| V_{H}H/V_{H}H-Fc | V_{H}H | V_{H}H-Fc | improvement |
| sdABP01 | 630 | 1.1 | 573 |
| sdABP02 | 17 | 1.8 | 9.5 |
| sdABP03 | 550 | 0.9 | 611 |
| sdABP04 | 18 | 1.1 | 16.5 |
| sdABP05 | 5.3 | 0.6 | 9 |
| sdABP06 | 0.96 | 0.6 | 1.6 |
| sdABP07 | 15 | 0.9 | 16.7 |
| sdABP08 | nd | 0.7 | nd |
| sdABP09 | nd | 0.9 | nd |

TABLE 9
Summary of apparent EC₅₀ values from FIG. 10.

EC₅₀ (nM)

| V_{H}H-Fc | Human DPEP-1 | Mouse DPEP-1 |
|---|---|---|
| sdABP05 | 1.4 | 3.9 |
| sdABP06 | 0.8 | 2.8 |

### Example 12. Epitope Typing of DPEP-1 V_{H}Hs by SDS-PAGE/Western Blot Analysis

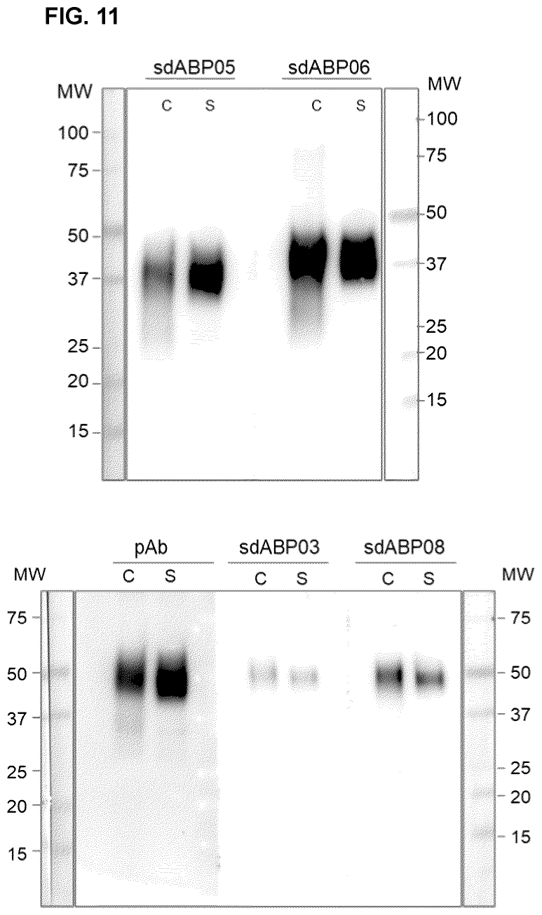

To determine, if V_{H}Hs recognize a linear or conformational epitope. V_{H}H-Fcs were subjected to epitope typing experiments by SDS-PAGE/western blot. One hundred ng of each recombinant human DPEP-1 per well (Creative biomart. Cat #DPEP1-77H or SinoBiologicals. Cat #13543-H08H) were separated on SDS-PAGE gels, transferred to PVDF membrane, probed with 100 ng of individual DPEP-1 V_{H}H-Fcs or anti-human-DPEP-1 rabbit pAb control for 1 h at room temperature. Membranes were washed five times and binding of V_{H}H-Fc was detected using 1 μg/mL of HRP-conjugated goat anti-human IgG (SIGMA, Cat #A0170) diluted in PBS/1% BSA. Binding of the pAb positive control was detected with goat anti-rabbit:HRP (Jackson Immunoresearch. Cat #111-035-144). After 1 h incubation, membranes were washed five times with PBS-0.05% Tween 20 and peroxidase activity was detected using chemiluminescent reagent (SuperSignal West Pico PLUS Chemiluminescent Substrate. Thermo Fisher, Cat #34580). Images were acquired with Molecular Imager R Gel Doc™ XR System (BioRad. Cat #1708195EDU). Epitope typing experiments indicated sdABP05, sdABP06, sdABP03 and sdABP08 V_{H}H-Fcs recognized linear epitopes with the remaining recognizing conformational epitopes (FIG. 11).

### Example 13. Human DPEP-1-Specific sdABP or V_{H}H-Fc Inhibits Metastasis in a Patient-Derived Lung Cancer Xenograft In Vivo

Patient-derived lung cancer xenograft is maintained by passage in severe combined immunodeficiency (SCID) mice. Stock tumors are harvested sterile and chopped into 1 mm sized specimens. Six specimens are implanted into the flanks of each SCID mouse. Tumors are continued to grow untreated until the size of the tumor is about 200 mm³. A sdABP or V_{H}H-Fc disclosed herein (sdABP01, 02, 03, 04, 05, 06, 07, 08, or 09, or V_{H}H-Fc thereof) or a control Ab (A20.1) (each at 10 mg/kg, 20 mg/kg, or 40 mg/kg) is administered twice 7 days apart by intravenous bolus injection. Dosage is determined based on the weight of individual animals weighed immediately prior to administration. Tumor growth is monitored by measuring caliper every 3-4 days. Tumor size is calculated as width 2× length/2, where width is the smallest size and length is the largest size value. sdABPs disclosed herein significantly prevent metastasis and inhibit the growth of patient-derived lung cancer xenografts implanted in SCID mice when compared to the control. The sdABPs disclosed herein are useful for reducing tumor burden and inhibits metastasis.

### Example 14. sdABP or VAH-Fc Inhibits Melanoma-Lung Metastasis in a Syngeneic Animal Model In Vivo

8-10 weeks old C57-BL6 mice (Charles River) are injected intravenously with 100,000 B16-F10 murine melanoma cells 5 minutes after the injection of a sdABP or V_{H}H-Fc disclosed herein (sdABP05, 06, or V_{H}H-Fc thereof) or a control Ab (A20.1) (each at 10 mg/kg, 20 mg/kg, or 40 mg/kg) via intravenous tail injection. Animals are sacrificed after 2 weeks and lungs are harvested. Tissues are processed for histology and hematoxylin-eosin staining is performed to assess tumor burden. The sdABPs disclosed herein, in particular sdABP05 and sdABP06, and their V_{H}H-Fc counterparts, are useful for reducing tumor burden and inhibits metastasis.

The present disclosure is not to be limited in scope by the specific embodiments described herein, since such embodiments are intended as but single illustrations of one aspect of the disclosure and any functionally equivalent embodiments are within the scope of this disclosure. Indeed, various modifications of the disclosure in addition to those shown and described herein will become apparent to those skilled in the art from the foregoing description and accompanying drawings. It will be appreciated how various modifications may be made without departing from the disclosure. Such modifications are intended to fall within the scope of the appended claims.

All publications, patents and patent applications referred to herein are incorporated by reference in their entirety to the same extent as if each individual publication, patent or patent application was specifically and individually indicated to be incorporated by reference in its entirety. The citation of any reference herein is not an admission that such reference is available as prior art to the instant disclosure.

1. A binding agent that binds to DPEP-1 comprising:
(i) SEQ ID NOs: 21, 22, and 23;
(ii) SEQ ID NOs: 24, 25, and 26;
(iii) SEQ ID NOs: 27, 28, and 29;
(iv) SEQ ID NOs: 30, 31, and 32;
(v) SEQ ID NOs: 33, 34, and 35;
(vi) SEQ ID NOs: 36, 37, and 38;
(vii) SEQ ID NOs: 39, 40, and 41;
(viii) SEQ ID NOs: 42, 43, and 44; or
(ix) SEQ ID NOs: 45, 46, and 47.

2. The binding agent of claim 1, comprising an amino acid sequence that is at least 75%, 80%, 85%, 90%, 95%, 97%, 98%, 99%, 99.5%, 99.9% or 100% identical to any one of SEQ ID NOs: 12-20 and 48-56.

3. The binding agent of claim 2, comprising the amino acid sequence of any one of SEQ ID NOs: 12-20 and 48-56.

4. The binding agent of claim 1, wherein the binding agent is a monoclonal, polyclonal, chimeric, humanized antibody, or antigen binding fragment thereof, optionally the binding agent is an antigen binding fragment fused to a Fc domain, optionally the binding agent is an antigen binding fragment, and wherein the antigen binding fragment is a Fv, scFv, Fab, Fab′, F(ab′)2, dsFv, ds-scFv, sdAB, dimer, minibody, diabody, or multimer antigen binding fragment, optionally the antigen binding fragment is sdAB, optionally the antibody or antigen binding fragment is human, mouse, llama, rabbit, sheep, or goat antibody or antigen binding fragment thereof.

5.-7. (canceled)

8. The binding agent of claim 1, wherein the antibody or antigen binding fragment comprises one or more amino acids selected from the group consisting of D-amino acids, modified amino acids, amino acid analogs or combinations thereof, optionally the modified amino acids comprise a modification selected from the group consisting of methylation, amidation, acetylation, and/or substitution with other chemical groups, optionally the antibody or antigen binding fragment is modified by pegylation, acetylation, glycosylation, biotinylation, or prenylation.

9.-11. (canceled)

12. A pharmaceutical composition comprising the binding agent of claim 1 and at least one pharmaceutical carrier.

13. A method for treating or preventing a disorder in a human subject in need thereof, comprising administering an effective among of the binding agent of claim 1 in the human subject.

14. The method of claim 13, wherein the disorder is selected from the group consisting of acute kidney injury, sepsis-induced condition, and tumor metastasis.

15. The method of claim 14, wherein the acute kidney injury comprises ischemia reperfusion-induced condition, pigment-induced condition, toxin-induced condition, or drug-induced condition.

16. The method of claim 14, wherein the sepsis-induced condition comprises bacterial or viral sepsis-induced condition, optionally the viral sepsis-induced condition comprises a COVID-19 sepsis-induced condition, optionally the sepsis-induced condition is associated with acute respiratory distress syndrome, encephalopathy, liver failure, kidney failure, or heart failure.

17.-18. (canceled)

19. The method of claim 14, wherein the tumor metastasis is associated with pancreatic cancer, kidney cancer, urogenital cancer, melanoma, prostate carcinoma, lung carcinoma, breast carcinoma, thyroid carcinoma, brain cancers, ovarian carcinomas, cervical cancer, uterine endometrial carcinoma, primary peritoneal carcinoma, mesothelioma, eye cancer, muscle, lymphoma, esophageal cancer, gastric cancer, liver cancer, small intestinal tumor, colon cancer, testicular cancer, skin cancers, or adrenal carcinoma.

20. The method of claim 19, wherein the kidney cancer is renal cell carcinoma (RCC), or wherein the urogenital cancer is urothelial carcinomas in urinary bladder, kidney, pelvic or ureter, or wherein the lung carcinomas is non-small cell carcinoma, small cell carcinoma, or neuroendocrine carcinoma, or wherein the breast carcinoma is ductal carcinoma, lobular carcinoma, or mixed ductal and lobular carcinoma, or wherein the thyroid carcinomas is papillary thyroid carcinoma, follicular carcinoma, or medullary carcinoma, or wherein the brain cancer is meningioma, astrocytoma, glioblastoma, cerebellum tumors, or medulloblastoma, or wherein the ovarian carcinoma is serous, mucinous, or endometrioid type, or wherein the cervical cancer is squamous cell carcinoma in situ, invasive squamous cell carcinoma, or endocervical adenocarcinoma, or wherein the uterine endometrial carcinoma is endometrioid, serous, or mucinous type, or wherein the mesothelioma is pleural or peritoneal, or wherein the eye cancer is retinoblastoma, or wherein the muscle cancer is rhabdosarcoma or leiomyosarcoma, or wherein the esophageal cancer is adenocarcinoma or squamous cell carcinoma, or wherein the gastric cancer is gastric adenocarcinoma or gastrointestinal stroma tumor, or wherein the liver cancer is hepatocellular carcinoma or bile duct cancer, or wherein the small intestinal tumor is small intestinal stromal tumor or carcinoid tumor, or wherein the colon cancer is adenocarcinoma of the colon, colon high grade dysplasia, or colon carcinoid tumor, or wherein the skin cancer is melanoma or squamous cell carcinoma.

21.-38. (canceled)

39. The method of claim 13, wherein the disorder is selected from the group consisting of inflammation, ischemia-reperfusion injury, and ischemia-reperfusion injury related disorder.

40. The method of claim 39, wherein the disorder is inflammation, optionally the inflammation is associated with an inflammatory disorder selected from the group consisting of gastritis, gout, gouty arthritis, arthritis, rheumatoid arthritis, kidney failure, lupus, asthma, psoriasis, pancreatitis, allergy, fibrosis, surgical complications, anemia, fibromyalgia, cancer, heart attack, congestive heart failure, stroke, aortic valve stenosis, arteriosclerosis, osteoporosis, multiple sclerosis, Alzheimer's disease, Parkinson's disease, ulcers, chronic bronchitis, asthma, allergy, acute lung injury, pulmonary inflammation, airway hyper-responsiveness, vasculitis, septic shock, inflammatory skin disorders, psoriasis, atopic dermatitis, eczema, and inflammatory bowel disease.

41. (canceled)

42. The method of claim 40, wherein the inflammatory bowel disease is Crohn's disease or ulcerative colitis.

43. The method of claim 39, wherein the ischemia-reperfusion injury related disorder is associated with ischemic and post-ischemic events in organs and tissues, and the disorder is selected from a group consisting of thrombotic stroke, myocardial infarction; angina pectoris, embolic vascular occlusions, peripheral vascular insufficiency, splanchnic artery occlusion, arterial occlusion by thrombi, arterial occlusion by embolisms, arterial occlusion by non-occlusive processes, mesenteric arterial occlusion, mesenteric vein occlusion, ischemia-reperfusion injury to the mesenteric microcirculation, ischemic acute renal failure, ischemia-reperfusion injury to the cerebral tissue, intestinal intussusception, hemodynamic shock, tissue dysfunction, organ failure, restenosis, atherosclerosis, thrombosis, platelet aggregation, shock liver, spinal cord injury, or brain injury, optionally the arterial occlusion by non-occlusive processes is arterial occlusion following low mesenteric flow or sepsis, optionally the organ failure is heart failure, liver failure, kidney failure, or the like.

44.-45. (canceled)

46. The method of claim 39, wherein the ischemia-reperfusion injury is resulted from a surgical procedure, optionally the surgical procedure is peri-operative procedure, cardiac surgery, organ surgery, organ transplantation, angiography, cardiopulmonary, or cerebral resuscitation.

47. (canceled)

48. The method of claim 39, wherein the ischemia-reperfusion injury is associated with harvesting donor organs for transplantation or wherein the ischemia-reperfusion injury occurs to allograft organs during donor procurement, ex vivo handling, or implantation into a transplant recipient.

49. (canceled)

50. A kit comprising the binding agent of claim 1, and instruction for use.


SEQUENCE LISTING (56 sequences)

SEQ ID NO: 1 - protein, 155 aa, Lama glama
EVQLVESGGGLVQPGGSLRLSCAASGSTLNWYTIGWFRQAPGKEREEVSCISSSGGSTKYADSVKGRFTISRSNALNTVYLQMNTLKPDDTAVYYCALDLDSAFCGSHISEYEYWGQGTQVTVSSGQAGQGGGLNDIFEAQKIEWHELEHHHHHH

SEQ ID NO: 2 - protein, 150 aa, Lama glama
EVQLVESGGGSVQAGGSLRLSCVASGIHFGSHSMAWYRQAPGKERDLVARISALGNTNYANSVKGRFTISRDTNKSTLYLQMNTLKPEDTAMYYCAPWSAYDREGDFRSWGQGTQVTVSSGQAGQGGGLNDIFEAQKIEWHELEHHHHHH

SEQ ID NO: 3 - protein, 159 aa, Lama glama
EVQLVESGGGLVQPGGSLRLSCATSEFTLDYYAIGWFRQAPGKEREGVSCISSSGGTTNYADSVKGRFTISSDNAKNTVSLQMNSLRPEDTAVYYCAAARVSAYYLGNYGCLNAEYGYWGQGTQVTVSSGQAGQGGGLNDIFEAQKIEWHELEHHHHHH

SEQ ID NO: 4 - protein, 152 aa, Lama glama
EVQLVESGGGLVQPGGSLRLSCAASGFTFSSYYMSWVRQAPGKGPEWVSGINTDGDDTSYADSVKGRFTISRDNAKNTLYLQMSSLKPEDTALYYCARAARSGSTTWGRNYWGQGTQVTVSSGQAGQGGGLNDIFEAQKIEWHELEHHHHHH

SEQ ID NO: 5 - protein, 148 aa, Lama glama
EVQLVESGGGLVQPGGSLRLSCAASGFTFSTYAMTWVRKAPGKGFEWISSIDSGGGVTLYADSVKGRFTISKDNAKNTLYLQMNNLKPDDTAVYYCVKNYGSTSLQSRGQGTQVTVSSGQAGQGGGLNDIFEAQKIEWHELEHHHHHH

SEQ ID NO: 6 - protein, 154 aa, Lama glama
EVQLVDSGGGLVQPGGSLRLSCAASGFTFSNYDMSWVRQGPGKGPEWVSIISYVGGLTRYSDSVKGRFTISRDNAKNTLYLQMNSLNTEDTALYYCARVKSMHPTSTTGEYDYRGRGTQVTVSSGQAGQGGGLNDIFEAQKIEWHELEHHHHHH

SEQ ID NO: 7 - protein, 158 aa, Lama glama
EVQLVESGGGLVEYGGSLRLSCAASESTLDNYAIAWFRQAPGKEREVVSCVGKSGGRSDYADSVKGRFTISRDNAKNTVYLQMNSLKPEDTAVYSCAARRVWFGGCVLGTSQGQYDYWGQGTQVTVSSGQAGQGGGLNDIFEAQKIEWHELEHHHHHH

SEQ ID NO: 8 - protein, 155 aa, Lama glama
EVQLVSSGGGLVQPGGSLRLSCKASRFTLERYTIGWFRQAPGKEREGIACISSSGGDTNYADSVKGRFTISRDNVVEKVYLQMDSLKPEDTAVYYCAARTYACDYKSRWLTYEFRGQGTQVTVSSGQAGQGGGLNDIFEAQKIEWHELEHHHHHH

SEQ ID NO: 9 - protein, 142 aa, Lama glama
QVQLVESGGGLVQPGGSLRLSCAASGFTFSSYGMTWVRQAPGKGFEWVSTISISGSRTTYAGSVKDRFTISRDNAKNTLYLQMNSLKPEDTAVYYCRNILVQGQGTQVTVSSGQAGQGGGLNDIFEAQKIEWHELEHHHHHH

SEQ ID NO: 10 - protein, 15 aa, Artificial Sequence
  Synthetic construct
GLNDIFEAQKIEWHE

SEQ ID NO: 11 - protein, 6 aa, Artificial Sequence
  Synthetic construct
HHHHHH

SEQ ID NO: 12 - protein, 125 aa, Lama glama
EVQLVESGGGLVQPGGSLRLSCAASGSTLNWYTIGWFRQAPGKEREEVSCISSSGGSTKYADSVKGRFTISRSNALNTVYLQMNTLKPDDTAVYYCALDLDSAFCGSHISEYEYWGQGTQVTVSS

SEQ ID NO: 13 - protein, 120 aa, Lama glama
EVQLVESGGGSVQAGGSLRLSCVASGIHFGSHSMAWYRQAPGKERDLVARISALGNTNYANSVKGRFTISRDTNKSTLYLQMNTLKPEDTAMYYCAPWSAYDREGDFRSWGQGTQVTVSS

SEQ ID NO: 14 - protein, 129 aa, Lama glama
EVQLVESGGGLVQPGGSLRLSCATSEFTLDYYAIGWFRQAPGKEREGVSCISSSGGTTNYADSVKGRFTISSDNAKNTVSLQMNSLRPEDTAVYYCAAARVSAYYLGNYGCLNAEYGYWGQGTQVTVSS

SEQ ID NO: 15 - protein, 122 aa, Lama glama
EVQLVESGGGLVQPGGSLRLSCAASGFTFSSYYMSWVRQAPGKGPEWVSGINTDGDDTSYADSVKGRFTISRDNAKNTLYLQMSSLKPEDTALYYCARAARSGSTTWGRNYWGQGTQVTVSS

SEQ ID NO: 16 - protein, 118 aa, Lama glama
EVQLVESGGGLVQPGGSLRLSCAASGFTFSTYAMTWVRKAPGKGFEWISSIDSGGGVTLYADSVKGRFTISKDNAKNTLYLQMNNLKPDDTAVYYCVKNYGSTSLQSRGQGTQVTVSS

SEQ ID NO: 17 - protein, 124 aa, Lama glama
EVQLVDSGGGLVQPGGSLRLSCAASGFTFSNYDMSWVRQGPGKGPEWVSIISYVGGLTRYSDSVKGRFTISRDNAKNTLYLQMNSLNTEDTALYYCARVKSMHPTSTTGEYDYRGRGTQVTVSS

SEQ ID NO: 18 - protein, 128 aa, Lama glama
EVQLVESGGGLVEYGGSLRLSCAASESTLDNYAIAWFRQAPGKEREVVSCVGKSGGRSDYADSVKGRFTISRDNAKNTVYLQMNSLKPEDTAVYSCAARRVWFGGCVLGTSQGQYDYWGQGTQVTVSS

SEQ ID NO: 19 - protein, 125 aa, Lama glama
EVQLVSSGGGLVQPGGSLRLSCKASRFTLERYTIGWFRQAPGKEREGIACISSSGGDTNYADSVKGRFTISRDNVVEKVYLQMDSLKPEDTAVYYCAARTYACDYKSRWLTYEFRGQGTQVTVSS

SEQ ID NO: 20 - protein, 112 aa, Lama glama
QVQLVESGGGLVQPGGSLRLSCAASGFTFSSYGMTWVRQAPGKGFEWVSTISISGSRTTYAGSVKDRFTISRDNAKNTLYLQMNSLKPEDTAVYYCRNILVQGQGTQVTVSS

SEQ ID NO: 21 - protein, 8 aa, Lama glama
GSTLNWYT

SEQ ID NO: 22 - protein, 8 aa, Lama glama
ISSSGGST

SEQ ID NO: 23 - protein, 18 aa, Lama glama
ALDLDSAFCGSHISEYEY

SEQ ID NO: 24 - protein, 8 aa, Lama glama
GIHFGSHS

SEQ ID NO: 25 - protein, 7 aa, Lama glama
ISALGNT

SEQ ID NO: 26 - protein, 14 aa, Lama glama
APWSAYDREGDFRS

SEQ ID NO: 27 - protein, 8 aa, Lama glama
EFTLDYYA

SEQ ID NO: 28 - protein, 8 aa, Lama glama
ISSSGGTT

SEQ ID NO: 29 - protein, 22 aa, Lama glama
AAARVSAYYLGNYGCLNAEYGY

SEQ ID NO: 30 - protein, 8 aa, Lama glama
GFTFSSYY

SEQ ID NO: 31 - protein, 8 aa, Lama glama
INTDGDDT

SEQ ID NO: 32 - protein, 15 aa, Lama glama
ARAARSGSTTWGRNY

SEQ ID NO: 33 - protein, 8 aa, Lama glama
GFTFSTYA

SEQ ID NO: 34 - protein, 8 aa, Lama glama
IDSGGGVT

SEQ ID NO: 35 - protein, 11 aa, Lama glama
VKNYGSTSLQS

SEQ ID NO: 36 - protein, 8 aa, Lama glama
GFTFSNYD

SEQ ID NO: 37 - protein, 8 aa, Lama glama
ISYVGGLT

SEQ ID NO: 38 - protein, 17 aa, Lama glama
ARVKSMHPTSTTGEYDY

SEQ ID NO: 39 - protein, 8 aa, Lama glama
ESTLDNYA

SEQ ID NO: 40 - protein, 8 aa, Lama glama
VGKSGGRS

SEQ ID NO: 41 - protein, 21 aa, Lama glama
AARRVWFGGCVLGTSQGQYDY

SEQ ID NO: 42 - protein, 8 aa, Lama glama
RFTLERYT

SEQ ID NO: 43 - protein, 8 aa, Lama glama
ISSSGGDT

SEQ ID NO: 44 - protein, 18 aa, Lama glama
AARTYACDYKSRWLTYEF

SEQ ID NO: 45 - protein, 8 aa, Lama glama
GFTFSSYG

SEQ ID NO: 46 - protein, 8 aa, Lama glama
ISISGSRT

SEQ ID NO: 47 - protein, 5 aa, Lama glama
RNILV

SEQ ID NO: 48 - protein, 357 aa, Artificial Sequence
  Synthetic construct
EVQLVESGGGLVQPGGSLRLSCAASGSTLNWYTIGWFRQAPGKEREEVSCISSSGGSTKYADSVKGRFTISRSNALNTVYLQMNTLKPDDTAVYYCALDLDSAFCGSHISEYEYWGQGTQVTVSSAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 49 - protein, 352 aa, Artificial Sequence
  Synthetic construct
EVQLVESGGGSVQAGGSLRLSCVASGIHFGSHSMAWYRQAPGKERDLVARISALGNTNYANSVKGRFTISRDTNKSTLYLQMNTLKPEDTAMYYCAPWSAYDREGDFRSWGQGTQVTVSSAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 50 - protein, 361 aa, Artificial Sequence
  Synthetic construct
EVQLVESGGGLVQPGGSLRLSCATSEFTLDYYAIGWFRQAPGKEREGVSCISSSGGTTNYADSVKGRFTISSDNAKNTVSLQMNSLRPEDTAVYYCAAARVSAYYLGNYGCLNAEYGYWGQGTQVTVSSAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 51 - protein, 354 aa, Artificial Sequence
  Synthetic construct
EVQLVESGGGLVQPGGSLRLSCAASGFTFSSYYMSWVRQAPGKGPEWVSGINTDGDDTSYADSVKGRFTISRDNAKNTLYLQMSSLKPEDTALYYCARAARSGSTTWGRNYWGQGTQVTVSSAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 52 - protein, 350 aa, Artificial Sequence
  Synthetic construct
EVQLVESGGGLVQPGGSLRLSCAASGFTFSTYAMTWVRKAPGKGFEWISSIDSGGGVTLYADSVKGRFTISKDNAKNTLYLQMNNLKPDDTAVYYCVKNYGSTSLQSRGQGTQVTVSSAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 53 - protein, 356 aa, Artificial Sequence
  Synthetic construct
EVQLVDSGGGLVQPGGSLRLSCAASGFTFSNYDMSWVRQGPGKGPEWVSIISYVGGLTRYSDSVKGRFTISRDNAKNTLYLQMNSLNTEDTALYYCARVKSMHPTSTTGEYDYRGRGTQVTVSSAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 54 - protein, 360 aa, Artificial Sequence
  Synthetic construct
EVQLVESGGGLVEYGGSLRLSCAASESTLDNYAIAWFRQAPGKEREVVSCVGKSGGRSDYADSVKGRFTISRDNAKNTVYLQMNSLKPEDTAVYSCAARRVWFGGCVLGTSQGQYDYWGQGTQVTVSSAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 55 - protein, 357 aa, Artificial Sequence
  Synthetic contruct
EVQLVSSGGGLVQPGGSLRLSCKASRFTLERYTIGWFRQAPGKEREGIACISSSGGDTNYADSVKGRFTISRDNVVEKVYLQMDSLKPEDTAVYYCAARTYACDYKSRWLTYEFRGQGTQVTVSSAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 56 - protein, 344 aa, Artificial Sequence
  Synthetic construct
QVQLVESGGGLVQPGGSLRLSCAASGFTFSSYGMTWVRQAPGKGFEWVSTISISGSRTTYAGSVKDRFTISRDNAKNTLYLQMNSLKPEDTAVYYCRNILVQGQGTQVTVSSAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG